SEQUENCE LISTING (42 sequences)

SEQ ID NO: 1 - DNA, 18 bp, Artificial sequence
  Synthetic oligonucleotide
gcctacgacgtaggcgcc

SEQ ID NO: 2 - protein, 6 aa, Artificial sequence
  Synthetic peptide
AYDVGA

SEQ ID NO: 3 - DNA, 21 bp, Artificial sequence
  Synthetic oligonucleotide
gtaatacgactcactataggg

SEQ ID NO: 4 - DNA, 52 bp, Artificial sequence
  Synthetic oligonucleotide
tttttgctgccggtcacttaacattatgctgagtgatatcccaaaaaccccc

SEQ ID NO: 5 - DNA, 40 bp, Artificial sequence
  Synthetic oligonucleotide
gtgtggtggtggtggtggtggtggtggtggtggtggtggt

SEQ ID NO: 6 - DNA, 50 bp, Artificial sequence
  Synthetic oligonucleotide
cacaccaccaccaccaccaccaccaccaccaccaccaccaaaaaattttt

SEQ ID NO: 7 - protein, 984 aa, Methanopyrus TAG11
VALVYDAEFVGSEREFEEERETFLKGVKAYDGVLATRYLIERSPSAKDDEELLELHQNFILLTGSYACSIDPTEDRYQNVIVRGVNFDERVQRLSTGGSPARYAIVYRRGWKAIAKALNIDEEDVPAIEVRAVKRNPLQPALYRILVRYGRVDLMPITVDEVPPEMAGEFERLIERYDVPINEKEERILEILRENPWTPHDEIARRLGLSVSEVEGEKDPESSGIYSLWSRVVVNIEYDERTAERHVKRRDRILEELYERLEELSERYLRRPLTRRWIVEHKRDIMRRYLEQRIVECALKLQDRYGIREDAALCLARTFDGSISMISTTPYRTLKDVCPDLTLEEAKSINRTLATLIDEHGLSPDAADELIENFESIAGILATDLKEIDQMHEEGRLSEEAYRAAVEIQLAELTKKGGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESVVGDLEKADELKRKYGSASAVRSLPVEELRELGFSDDEIAEIKGVPKKLREAFDLETAAELYEQYGSLKEIGRRLSYDDLLELGATPKAAAEIKGPEFKFLLNIEGVGPELAERILEAVDYDLERLASMNPEELEEKVKGLGEELAERVVYAARERVESRRKSGRQERSEEEWKEWLERKVGEGRSRRLIEYFGSAGEVGKLAENAEVSKLLEVPGIGDEAVARLVPGYKTLRDAGLTPVEAERVLKRYGSVSKVQEEATPDELRELGLSDAKIARILGLRSLVNKGLDVDTAYELKRRYGSVSAVRKAPVKALRELGLSDRKIARIKSIPETMLQVRGMSVEKAERLLERFDTWTKVKEAPVSELVKVPGVGLSLVKEIKAQADPAWKALLDVKGVSPELADRLVEEFGSPYRVLTAKKSDLMKVDGVGPKLAKRIRTAGKRYVEERRSRRERIRRKLRG

SEQ ID NO: 8 - DNA, 2955 bp, Methanopyrus TAG11
gtggctctggtgtacgacgctgagttcgtgggctcggagcgagagttcgaggaagaacgcgagacgttcctcaaaggtgtcaaggcttacgacggcgttctggccacccgttacttgatagagaggtctcctagcgcaaaggacgacgaggagctgttagagcttcaccagaatttcatccttctcacgggatcgtacgcctgctcgatagatccgacagaagataggtatcaaaacgtcatagttcgtggtgttaacttcgatgaacgagttcaacgcctgtccacgggcggttcaccggctcgctacgcgatcgtgtacaggcgtggctggaaggcgatcgccaaggccttgaatatcgacgaggaagacgttcctgccatagaggtgcgtgctgtgaaacgcaacccactccagccggcattgtaccggatcctggtacgatacgggcgcgtcgacctaatgcccataaccgtggacgaggtgccacctgagatggccggcgagttcgagcgactgatcgaacggtacgacgtcccgatcaacgagaaggaggagcgcatactcgagatcctcagggagaacccatggaccccacacgacgaaatcgcgagacggctcgggctctcggtttcggaagtcgagggtgagaaggatccggagagcagcggtatctacagcctgtggtctcgagtcgtcgtgaacatcgagtacgacgagcgtacggccgagcgacacgttaagcgccgggatcgaattctcgaagaactatacgagcgcctggaggagctctcggagaggtacttacgtcgtccgttgactagacggtggatcgtcgaacataagcgcgacatcatgagaaggtacctcgagcagcggatcgtcgagtgtgcgctcaagctccaggaccgttacgggatccgtgaggatgcggcactgtgtctcgcaaggactttcgacgggtccatctcaatgatctctaccactccgtaccggacgctcaaggacgtgtgccccgacctgacactcgaggaggccaagtccatcaaccgcaccctggcgacactgatcgacgagcacggtctcagccccgacgccgcggacgagctcatcgagaacttcgaatcgatcgccggtattttggctaccgacttgaaggagatagatcaaatgcacgaggaaggaaggctatccgaggaggcttaccgggccgccgtcgagatacagctggcagagctcacgaagaagggaggtgtgggtaggaagactgcggagcgtctcttacgcgccttcggaaaccccgaacgcgtcaagcagctggcccgcgagttcgagatcgagaagctagcctcggtggaaggggtcggcgagcgcgtcctacgcagtctcgtcccggggtacgcttcgctgatctcaatccgtggcatcgaccgggagcgggcggagcgtctgctcaagaagtacggcggctactccaaggtccgtgaggccggcgtcgaagagttgcgcgaggacggcctcaccgacgcccaaatccgggagctcaagggtctaaagaccctcgagagcgtagtaggggatttagaaaaggccgacgaactaaagcggaagtacggatccgcgtccgcggttcgaagtctacccgtagaggagctgcgcgaactcgggttctccgacgacgaaatcgccgagattaaaggagtacctaagaagctccgggaggccttcgacctcgagaccgccgcggaactctacgagcagtacggttcgctgaaggagatcggtcgtcgattatcttacgacgatctactcgagctcggtgcgactccgaaggccgcagctgagatcaagggaccggagttcaagttcctcttgaacatcgaaggggtcggaccggaactcgccgagcggatactcgaggcagtggattacgacctcgagcgattggcttccatgaatccagaggaactcgaggagaaagtgaaaggactgggtgaagaactcgcggaacgcgtcgtgtacgccgctagggagcgcgtagaaagtcgcagaaagtccggccgccaggagcgatcggaggaagaatggaaggagtggctcgagcgtaaggtcggtgaggggagatctcgccggctgatcgagtatttcggatccgcgggtgaggtaggaaaactggccgagaacgccgaagtatcgaagctgctggaggtccctggtataggcgacgaggccgtcgccaggctcgtaccgggttataagaccctacgagacgccggtctcacaccggttgaagcggagcgcgtgctgaaacggtacgggtcggtctccaaagtccaagaagaagccactccggacgagttacgcgaactcggcctcagcgacgccaagatcgcgaggatcctgggcctgcgtagcctggtgaataagggcctggacgtggacaccgcgtacgagctcaagcgtagatacggttccgtctccgccgtacggaaggcaccggtgaaagcactgcgcgagctcggcctctccgatcgtaagatcgcgcgtatcaagagtatcccggaaactatgctccaggtacgagggatgagtgtggagaaagcggagcggctactggagcgtttcgatacctggactaaggtgaaggaagcacccgtatcggagttggtgaaagttccgggcgtcggattgagtttggtcaaggagatcaaggctcaagcggacccggcctggaaggcactcttagacgtcaaaggggtcagtccggagctggccgaccgactcgtcgaggaattcggcagcccctaccgggtgctgacggccaaaaaatccgacctgatgaaggtcgacggagtcgggccgaagctcgccaagcgaatccggaccgcgggcaaacgatacgtggaggagcgtaggtcgaggagggagaggatcaggaggaagctccgaggatga

SEQ ID NO: 9 - protein, 984 aa, Methanopyrus kandleri top5
VALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 10 - DNA, 2951 bp, Methanopyrus kandleri top5
gtggcgctggtgtacgacgctgagttcgtgggctcggagcgagagttcgaggaggaacgcgagacgttcctcaagggtgtcaaggcctacgacggcgttctggccacccgttacttgatggagagatcttccagcgcgaagaacgacgaggagctgctcgagcttcaccagaatttcatcctcctcacgggatcgtacgcctgctcgatagacccgaccgaagataggtatcagaacgtcatagtccgtggcgttaacttcgatgaacgagttcaacgcctgtccacgggcggttcaccggcccgctacgcgatcgtgtacaggcgtggctggagggcgatcgccaaggctctagatatcgacgaggaggacgtcccggccatagaggtgcgtgccgtgaagcgtaacccgctccaaccggcgctgtaccggatcctggtgcgatacggacgcgtcgacctcatgcccgtaaccgtggacgaggtaccaccggagatggccggtgagttcgagcgactgatcgaacggtacgacgtcccgatcgacgagaaggaggagcgcatactcgagatcctcagggagaacccgtggactccccatgacgagatcgcgagacggctcgggctctcggtctcggaagtcgagggtgagaaggacccggagagcagcggtatctacagcctgtggtctcgggtcgtcgtgaacatcgagtacgacgagcgtacggccaagcggcacgtcaagcgccgggatcgactgctcgaggaactgtacgagcacctggaggagctttcggagcggtacttacgtcatccgttgactagacggtggatcgtcgagcataagcgcgacatcatgagaaggtatctcgagcagcggatcgtcgagtgtgcgctcaagctccaggaccgttacgggatccgcgaggacgtggcgctgtgtctcgccagggccttcgacgggtccatctcgatgatcgctaccactccgtaccggacgctcaaggacgtgtgtcccgacctgacgctcgaggaggccaagtccgtcaaccgcaccctggcgacgctgatcgacgagcacggtctcagccccgacgccgcggacgaactcatcgagcacttcgagtcgatcgccggtattctggccaccgacctggaggagatcgagcgaatgtacgaggagggcaggctctccgaggaggcttaccgggccgccgtcgagatacagttggcggagctcacgaagaaggagggcgtgggtaggaagaccgcggagcgtctcttacgcgccttcggaaaccccgagcgcgtcaagcagctggcccgcgagttcgagatcgagaagctggcctcggtggaaggggtcggcgagcgcgtcctacgcagtctcgtcccggggtacgcttcgttgatctcgatcaggggcatcgacagggagcgggcggagcgtctgctcaagaagtacggtgggtactccaaggtccgtgaggctggtgtcgaagagctgcgcgaggacggcctcaccgacgctcaaatccgggagctcaagggtctgaagaccctggagagcatagtaggggatctggagaaggccgacgagctgaagcggaagtacggatccgcgtccgcggttcgacgtctgcccgtagaggagctacgcgaactcgggttctccgacgatgagatcgccgagatcaaggggatacctaagaagctccgggaggccttcgaccttgagaccgccgcggaactctacgagcggtacggttcgctgaaagagatcggtcgccgactctcttacgacgatctactcgagctcggtgcgactccgaaggccgcggccgagatcaaggggccggagttcaagttcctcctgaacatcgaaggggtcggaccgaaactcgctgagcggatactcgaggccgtggattatgacctcgagcgactggcttccctgaatcccgaggaacttgcggagaaggtggaaggactgggcgaagagctcgcggagcgcgtcgtgtacgctgctagggagcgcgtagaaagtcgcaggaagtccggccgccaggagcggtcggaggaagaatggaaggagtggctcgagcgtaaggtcggcgaggggagggctcgccggttgattgagtatttcggctccgcgggtgaagtaggaaagctggtcgagaacgccgaggtgtcgaagctactggaggtcccgggtataggcgacgaggccgtcgctaggctcgtaccgggctacaagaccctacgagacgccggtctcacgccggccgaagcggagcgcgtgctgaaacggtacggctcggtctccaaagtgcaggaaggagccactccggacgagttacgcgagctcggcctcggcgacgccaagatcgcgaggatcctgggcctgcgcagcctggtgaacaagaggctggacgtggacaccgcgtacgagctcaagcgtagatacggttccgtctccgccgtccggaaggccccggtgaaagaactgcgcgagctcggcctctccgatcggaagatcgcacgtatcaagggcatcccggagacgatgcttcaggtccgagggatgagcgtggagaaagcggagcggctgctggagcgtttcgatacctggaccaaggtgaaggaagctcccgtctcggagctggtgagagtcccgggtgtcggattgagtttggtgaaggagatcaaggctcaggtggatccggcctggaaggcacttctggatgtcaaaggggtcagtccggagctggccgaccggctcgtcgaggagctcggcagcccgtatcgggtgctgacggccaagaaatccgacctgatgagagtcgagagagtcggaccgaagctcgccgagcgaatccgggccgcgggcaaacggtacgtggaggagcgtaggtcgaggagggagaggatcaggaggaagctccgagg

SEQ ID NO: 11 - protein, 354 aa, Artificial sequence
  Chimeric protein
HMSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRGAYDVGAPKALEEAPWPPPEGAFVGFVLSRKEPMWADLLALAAARGGRVHRAPE

SEQ ID NO: 12 - protein, 337 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPE

SEQ ID NO: 13 - DNA, 18 bp, Artificial sequence
  Synthetic oligonucleotide
caggaaacagctatgacc

SEQ ID NO: 14 - protein, 33 aa, Methanopyrus TAG11
GSISMIATTPYRTLKDVCPDLTLEEAKSVNRTL

SEQ ID NO: 15 - protein, 33 aa, Homo sapiens
KIDEFLATGKLRKLEKIRQDDTSSSINFLTRVS

SEQ ID NO: 16 - protein, 51 aa, Methanopyrus TAG11
LIDEHGLSPDAADELIEHFESIAGILATDLEEIERMYEEGRLSEEAYRAAV

SEQ ID NO: 17 - protein, 51 aa, Thermus filiformis
ALGLPGVGEVLARNLARRFGTMDRLLEASLEELIEVEEVGELTARAILETL

SEQ ID NO: 18 - protein, 59 aa, Methanopyrus TAG11
QLAELTKKEGVGRKTAERLLRAFGNPERVKQLAREFEIEKLASVEGVGERVLRSLVPGY

SEQ ID NO: 19 - protein, 58 aa, Escherichia coli
LFKELIKTNGVGPKLALAILSGMSAQQFVNAVEREEVGALVKLPGIGKKTAERLIVEM

SEQ ID NO: 20 - protein, 42 aa, Methanopyrus TAG11
ASLISIRGIDRERAERLLKKYGGYSKVREAGVEELREDGLTD

SEQ ID NO: 21 - protein, 43 aa, Thermus filiformis
LYALGLPGVGEVLARNLARRFGTMDRLLEASLEELIEVEEVGE

SEQ ID NO: 22 - protein, 35 aa, Methanopyrus TAG11
GDLEKADELKRKYGSASAVRRLPVEELRELGFSDD

SEQ ID NO: 23 - protein, 36 aa, Thermus filiformis
GVGEVLARNLARRFGTMDRLLEASLEELIEVEEVGE

SEQ ID NO: 24 - protein, 39 aa, Methanopyrus TAG11
AELYERYGSLKEIGRRLSYDDLLELGATPKAAAEIKGPE

SEQ ID NO: 25 - protein, 39 aa, Bacillus stearothermophilus
IDKLVRYAADHELSLFEALGELEMIGIGAKAAGALAAFR

SEQ ID NO: 26 - protein, 59 aa, Methanopyrus TAG11
FKFLLNIEGVGPKLAERILEAVDYDLERLASLNPEELAEKVEGLGEELAERVVYAARER

SEQ ID NO: 27 - protein, 60 aa, Escherichia coli
FKELIKTNGVGPKLALAILSGMSAQQFVNAVEREEVGALVKLPGIGKKTAERLIVEMKDR

SEQ ID NO: 28 - protein, 39 aa, Methanopyrus TAG11
IEYFGSAGEVGKLVENAEVSKLLEVPGIGDEAVARLVPG

SEQ ID NO: 29 - protein, 39 aa, Escherichia coli
ILSGMSAQQFVNAVEREEVGALVKLPGIGKKTAERLIVE

SEQ ID NO: 30 - protein, 40 aa, Methanopyrus TAG11
YKTLRDAGLTPAEAERVLKRYGSVSKVQEGATPDELRELG

SEQ ID NO: 31 - protein, 40 aa, Escherichia coli
VAALVALGYKPQEASRMVSKIARPDASSETLIREALRAAL

SEQ ID NO: 32 - protein, 53 aa, Methanopyrus TAG11
LRSLVNKRLDVDTAYELKRRYGSVSAVRKAPVKELRELGLSDRKIARPIKGIP

SEQ ID NO: 33 - protein, 52 aa, Thermus filiformis
LYALGLPGVGEVLARNLARRFGTMDRLLEASLEELIEVEEVGELTARAILET

SEQ ID NO: 34 - protein, 49 aa, Methanopyrus TAG11
IPETMLQVRGMSVEKAERLLERFDTWTKVKEAPVSELVRVPGVGLSLVK

SEQ ID NO: 35 - protein, 49 aa, Thermus filiformis
RLLYALGLPGVGEVLARNLARRFGTMDRLLEASLEELIEVEEVGELTAR

SEQ ID NO: 36 - protein, 31 aa, Methanopyrus TAG11
AWKALLDVKGVSPELADRLVEELGSPYRVLT

SEQ ID NO: 37 - protein, 31 aa, Homo sapiens
RLLYALGLPGVGEVLARNLARRFGTMDRLLE

SEQ ID NO: 38 - protein, 44 aa, Methanopyrus TAG11
AKKSDLMRVERVGPKLAERIRAAGKRYVEERRSRRERIRRKLRG

SEQ ID NO: 39 - protein, 45 aa, Homo sapiens
SSINFLTRVSGIGPSAARKFVDEGIKTLEDLRKNEDKLNHHQRIG

SEQ ID NO: 40 - protein, 136 aa, Thermus filiformis
CPAKRFEAIRHYASRKAMDIEGLGEKLIERLLEKGLVRDVADLYHLRKEDLLGLERMGEKSAQNLLRQIEESKHRGLERLLYALGLPGVGEVLARNLARRFGTMDRLLEASLEELIEVEEVGELTARAILETLKDP

SEQ ID NO: 41 - protein, 126 aa, Rattus norvegicus
LANFEKNVSQAIHKYNAYRKAASVIAKYPHKIKSGAEAKKLPGVGTKIAEKIDEFLATGKLRKLEKIRQDDTSSSINFLTRVTGIGPSAARKLVDEGIKTLEDLRKNEDKLNHHQRIGLKYFEDFE

SEQ ID NO: 42 - protein, 135 aa, Escherichia coli
FNNKQERTLFKELIKTNGVGPKLALAILSGMSAQQFVNAVEREEVGALVKLPGIGKKTAERLIVEMKDRFKGLHGDLFTPAADLVLTSPASPATDDAEQEAVAALVALGYKPQEASRMVSKIARPDASSETLIRE

(19) United States
(12) Patent Application Publication
(10) Pub. No.: US 2006/0234227 A1
(43) Pub. Date: Oct. 19, 2006

(54) Helix-hairpin-helix motifs to manipulate properties of dna processing enzymes

(76) Inventors: Alexei Slesarev, Boyds, MD (US); Andrey Pavlo, Gaithersburg, MD (US); Sergei Kozyavkin, Germantown, MD (US); Nadezhda Pavlova, Gaithersburg, MD (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/514,342

(22) PCT Filed: May 14, 2003
(86) PCT No.: PCT/US03/15336

§ 371 Date: Apr. 27, 2006

Related U.S. Application Data
(60) Provisional application No. 60/380,423, filed on May 14, 2002.
(60) Provisional application No. 60/410,974, filed on Sep. 16, 2002.

(51) Int. Cl. C12Q 1/68; C12P 19/34; C12N 9/22

(52) U.S. Cl. 435/6; 435/199; 435/91.2

(57) ABSTRACT

The utility of Topo V's HhH motifs for modulating DNA binding properties of a DNA processing enzyme, such as Taq DNA polymerase or PfuDNA polymerase, is demonstrated. In one embodiment, an amino acid residue comprising one or more HhH domains derived from Topoisomerase V, linked to either the NH₂-terminus or COOH-terminus of a Taq polymerase fragment significantly broadens the processivity and/or salt concentration range of polymerase activity. The specific activities of the chimeric polymerases are not affected by added HhH motifs. Depending on the type of the construct, the thermal stability of chimeric polymerases increases or remains the same as that of Taq DNA polymerase or its Stoffel fragment. This invention further provides a method of increasing the salt tolerance of Taq polymerases. The methods of this invention may be applied to all pol A-type DNA polymerases, as well as to other DNA processing enzymes.

This work was supported in part by U.S. Department of Energy and National Institutes of Health grants (DE-FG02-98ER82577, 00ER83009, R44M55485, R43HG02186) to S. Kozyavkin and A. Slesarev.

### BACKGROUND OF THE INVENTION

1. Field of the Invention

The present invention provides a method of transferring the enzymatic properties of HhH domains of proteins to DNA processing enzymes. More specifically, one embodiment of this invention provides a method of increasing the ability of Taq polymerase fragments to work in high salt concentrations and/or increasing the processivity of Taq polymerase fragments by linking one or more helix-hairpin-helix (HhH) domains of topoisomerase V to said fragments. Another embodiment of this invention provides a method of increasing the ability of Pfu DNA polymerase to work in high salt concentrations and/or increasing the processivity of Pfu polymerase by linking one or more helix-hairpin-helix (HhH) domains of topoisomerase V to said polymerase. This invention further provides improved Taq and Pfu polymerases obtained by the method of this invention.

2. Description of the State of Art

Many gene regulatory proteins contain small, discrete structural motifs that utilize either α-helices or β-strands to bind the grooves of DNA. Among these are the helix-turn-helix, zinc finger, leucine zipper and helix-loop-helix motifs [1]. These may arise in different molecular contexts, and this is believed to occur either because of divergent evolution via gene duplication and insertion, or because of structural convergence via the effects of selective pressures on protein function.

Unlike gene regulatory proteins, other molecules that bind DNA do so in a non-sequence-specific manner. These proteins belong to a variety of protein families exemplified by nucleases, N-glycosylases, ligases, and helicases that are essential for the protein-mediated synthesis and DNA repair. These proteins have recently been shown to possess a common structural motif, called helix-hairpin-helix (HhH) [2, 3]. Doherty et al. [4] have analyzed the structure of individual HhH motifs in several protein families and concluded that HhH motifs exist as separate units. Shao et al. [5] have shown that HhH motifs are typically involved in the formation of a larger structure of five α-helices named (HhH)₂ domain. (HhH)₂ is a pseudo-2-fold unit composed of two HhH motifs linked by a connector α-helix. This compact structure with a well-defined hydrophobic core mirrors the symmetry of the DNA-double helix and facilitates strong DNA-binding properties. Protein-DNA contacts do not involve DNA bases but rather a sugar-phosphate chain. This allows proteins containing HhH motif to bind DNA in the non-sequence-specific manner.

A unique number of HhH motifs have been identified in DNA topoisomerase V (Topo V) [6-8]. The 684 C-terminal amino acids (out of total 984 amino acids) are organized into 12 repeats of about 50 amino acids each. All repeats consist of two similar HhH motifs. It has also been demonstrated that Topo V proteins lacking different parts of HhH superdomain remain fully active in relaxation of supercoiled DNA but become sensitive to salts [9, 10]. Thus, HhH motifs play a crucial role in Topo V interactions with DNA, which anchors the enzyme on DNA at high salt concentrations. Furthermore, these motifs confer high processivity on Topo V in a very broad range of salt concentrations [6-8].

Taq DNA polymerase belongs to the class of DNA polymerase I (pol A) enzymes that serve as model systems for studying the mechanisms of polymerase function [11]. These enzymes have multidomain structures, which include a polymerase domain, a 3′ to 5′ exonuclease domain, and a 5′ to 3′ exonuclease domain. In addition, they may contain a separate domain responsible for processivity [11]. Pfu DNA polymerase belongs to the class of DNA polymerase II (pol AI) enzymes. It is a high fidelity DNA polymerase, which has a 3′ to 5′ exonuclease activity. Taq and Pfu DNA polymerase are also of enormous practical interest because they are used extensively in polymerase chain reactions (PCR) and for DNA sequencing.

Processivity is a measurement of the ability of a DNA polymerase to incorporate one or more deoxynucleotides into a primer template molecule without the DNA polymerase dissociating from that molecule. DNA polymerases having low processivity, such as the Klenow fragment of DNA polymerase I of E. coli or Pfu DNA polymerase from Pyrococcus furiosus, will dissociate after about 5-40 nucleotides are incorporated on average.

Wang, in PCT publication WO 01/92501 A1, describes a general method of linking a sequence-non-specific nucleic-acid-binding domain to enzymes in a manner that enhances the ability of the enzyme to bind and catalytically modify the nucleic acid. Specifically disclosed are a Thermus or a Pyrococcus polymerase domain. However, Wang does not demonstrate whether other nucleic acid-modifying domains could be linked to particular enzymes and could further successfully enhance the properties of the enzymes.

Bedford, et al. in U.S. Pat. No. 5,972,603, describe a chimeric DNA polymerase with modified processivity. However, the chimeric protein comprises a DNA polymerase domain, wherein a processivity factor binding domain of a different DNA polymerase is substituted for a portion of the wild-type DNA polymerase.

### SUMMARY OF THE INVENTION

The present invention provides a method of transferring the enzymatic properties of HhH domains of proteins to DNA processing enzymes. This invention further provides novel chimeric proteins that are particularly well adapted for use in amplification reactions (such as the polymerase chain reaction) and related reactions, as well as in DNA sequencing. The processivity of a DNA processing enzyme or a fragment thereof can be significantly increased by linking an existing DNA processing enzyme or a fragment thereof to a non-naturally associated amino acid sequence comprising one or more helix-hairpin-helix (HhH) motifs. In addition, the non-naturally associated DNA binding site increases the salt tolerance of the chimeric protein in amplification reactions.

Those in the art will recognize that many equivalent chimeric molecules can be created using the teachings of this invention. Thus, for example, thermostable DNA polymerases can be created having increased processivity. In one embodiment, the invention provides pol A- and pol B-type DNA polymerases which are linked to an HhH-containing DNA binding domain. However, this invention further contemplates other pol A- and pol B type polymerases (such as those present in Thermus aquaticus and Pyrococcus furiosus) which can be modified to include such DNA binding domains according to this invention. In this way, a thermostable processive DNA polymerase can be created. Such a polymerase will have advantages over existing polymerases in DNA sequencing and amplification reactions.

Accordingly, one aspect of this invention provides a chimeric DNA comprising a DNA processing enzyme, or a fragment thereof, linked to an amino acid sequence derived from Topoisomerase V and comprising one or more helix-hairpin-helix (HhH) motifs not naturally associated with said processing enzyme or said fragment. In one embodiment, the DNA processing enzyme is a DNA polymerase or a fragment thereof having a DNA polymerase domain.

In another aspect, this invention provides a method for increasing the processivity of a DNA processing enzyme or a fragment thereof by linking said processing enzyme or said fragment to an amino acid sequence comprising one or more helix-hairpin-helix (HhH) domains of topoisomerase V. In one embodiment, the DNA processing enzyme is a DNA polymerase or a fragment thereof having a DNA polymerase domain.

Another aspect of this invention provides a method of transferring the enzymatic properties of DNA topoisomerase V (Topo V) to other DNA processing enzymes. More specifically, one embodiment of this invention provides a method of increasing the ability of Taq and Pfu polymerases to work in high salt concentrations and/or increasing the processivity of Taq and Pfu polymerases by linking an amino acid sequence comprising one or more helix-hairpin-helix (HhH) domains of topoisomerase V to said enzymes or fragments thereof. This invention further provides improved Taq and Pfu polymerases obtained by the method of this invention.

For example, this invention demonstrates that, if properly positioned, HhH repeats derived from Topo V not only restore the processivity of the Stoffel fragment of Taq polymerase and Pfu polymerase or fragments thereof to the level of Taq polymerase and Pfu polymerase, respectively, but also confer processivity on hybrid polymerases in high salts where Taq and Pfu polymerases works distributively and/or are inhibited.

Accordingly, this invention comprises a method of increasing the ability of DNA processing enzymes or a fragments thereof to work in high salt concentrations, wherein the method comprises linking an amino acid sequence comprising one or more HhH domains derived from Topo V to either the N-terminus or the C-terminus of the DNA processing enzyme or a fragment thereof to form a chimeric polymerase. In one embodiment, the DNA processing enzyme fragment is the Stoffel fragment of Taq polymerase. In another embodiment, the DNA processing enzyme is Pfu polymerase or fragments thereof.

Another aspect of this invention comprises a method of increasing the processivity of DNA processing enzyme or fragment thereof, wherein the method comprises linking one or more HhH domains derived from Topo V to either the N-terminus or the C-terminus of the DNA processing enzyme or fragments to form a chimeric polymerase. In one embodiment, the DNA processing enzyme fragment is the Stoffel fragment of Taq polymerase. In another embodiment, the DNA processing enzyme is Pfu polymerase or a fragment thereof.

In even further related aspects, the invention features improved methods for amplification or DNA sequencing of a nucleic acid, the improvement being the use of a chimeric DNA processing enzyme of this invention. The chimera comprises a DNA polymerase linked to an amino acid sequence comprising one or more helix-hairpin-helix (HhH) motifs not naturally associated with the DNA processing enzyme or fragment thereof, wherein the amino acid sequence is derived from Topoisomerase V.

This invention further provides a method of amplifying a nucleic acid, comprising combining said nucleic acid with a chimeric DNA polymerase comprising a DNA processing enzyme or a fragment thereof linked to an amino acid sequence derived from Topoisomerase V and comprising one or more helix-hairpin-helix (HhH) motifs not naturally associated with said DNA processing enzyme or said fragment, wherein said nucleic acid and said chimeric DNA polymerase are combined in an amplification reaction mixture under conditions that allow for amplification of nucleic acids. The amplification reactions work well under both isothermal and thermal cycling conditions, and further can be conducted in high salt concentrations.

Certain HhH were found to significantly broaden the salt concentration range of the polymerase activity of DNA polymerase fragments. The specific activities of the chimeric polymerases were not affected by added HhH motifs, yet their processivity increases. Depending on the type of the construct and buffer conditions used, the thermal stability of chimeric polymerases increases or remains the same as that of Taq DNA polymerase (or its Stoffel fragment) or the Pfu DNA polymerase. The method disclosed herein for raising the salt tolerance and/or processivity of Taq and Pfu polymerases may be applied to all pol A- and pol B-type DNA polymerases as well as to other DNA processing enzymes.

Additional advantages and features of this invention shall be set forth in part in the description and examples that follow, and in part will become apparent to those skilled in the art upon examination of the following or may be learned by the practice of the invention and in combinations particularly pointed out in the appended claims.

### BRIEF DESCRIPTION OF THE FIGURES

FIG. 1 provides schematic illustrations of the TopoTaq, TaqTopoC1, TaqTopoC2 and TaqTopoC3 chimeras formed by the method of this invention, where the HhH domains derived from Topo V are represented by the letters A-L.

FIG. 2 is a graph plotting the relative rate of primer extension for Taq DNA polymerase, Taq DNA polymerase fragments, and the TaqTopo V chimeras versus concentration of NaCl.

FIG. 3 is a graph plotting the relative rate of primer extension for Taq DNA polymerase, Taq DNA polymerase fragments, and the TaqTopo V chimeras versus concentration of KCl.

FIG. 4 is a graph plotting the relative rate of primer extent ion for Taq DNA polymerase, Taq DNA polymerase fragments, and the TaqTopo V chimeras versus concentration of potassium glutamate.

FIG. 5 is a graph plotting the relative rate of primer extent ion for PfuPol and PfuC2 in NaCl, KCl or KGlu versus concentration of the salt.

FIG. 6 is a graph plotting the initial rate of primer extension TopoTaq in μM/min versus concentration of TopoTaq with or without NaCl and/or betaine.

FIG. 7 is a graph plotting the initial rate of primer extension TopoTaq in μM/min versus concentration of PTJ in the presence of NaCl with or without betaine.

FIG. 8 is a graph plotting the processivity of Taq DNA polymerase, Taq DNA polymerase fragments, and the TaqTopo V chimeras versus concentration of NaCl.

FIG. 9 is a graph plotting the processivity of Taq DNA polymerase, Taq DNA polymerase fragments, and the TaqTopo V chimeras versus concentration of KCl.

FIG. 10 is a graph plotting the processivity of Taq DNA polymerase, Taq DNA polymerase fragments, and the TaqTopo V chimeras versus concentration of potassium glutamate.

FIG. 11 is a graph plotting the processivity of PfuPol and PfuC2 in NaCl, KCl or KGlu versus concentration of the salt.

FIG. 12 is a graph of the thermostability of Taq DNA polymerase, Taq DNA polymerase fragments, and the TaqTopo V chimeras at 95° C. plotted as activity (a.u.) versus time in minutes.

FIG. 13 is a graph of the thermostability of TopoTaq at 100° C. plotted as activity (a.u.) versus time in minutes.

FIG. 14 is a graph of the thermostability of Taq DNA polymerase, Taq DNA polymerase fragments, and the TaqTopo V chimeras at 100° C. plotted as activity (a.u.) versus time in minutes.

FIG. 15 is a graph of the thermostability of Pfu and PfuC2 at 100° C. plotted as activity (a.u.) versus time in minutes.

FIG. 16 is a urea gel picture of single-stranded DNA M13mp18(+) sequencing with ALF M13 Universal fluorescent primer using Taq DNA polymerase and N-TopoTaq. Salt concentrations are indicated on the gel.

FIG. 17 is a graph of the initial rates of primer extension reactions versus reaction temperature for enzymes with Taq polymerase catalytic domain.

FIG. 18 is a graph of the initial rates of primer extension reactions versus reaction temperature for enzymes with Pfu polymerase catalytic domain.

FIGS. 19 A-L are 3D models of TopoV HhH domains. Structural diagrams for the models of HhH domains A-L are shown along with the structural alignment of the TopoV domains and HhH domains in the proteins with the solved X-ray structures.

FIG. 19 M shows the calculated distribution of partial charge along the TopoV HhH domains at pH 7.0.

FIG. 20A shows the structural alignment of NAD⁺-dependent DNA ligase from Thermus filiformis, human DNA polymerase β.

FIG. 20B shows the structural alignment of holiday junction DNA helicase RuvA from Escherichia coli and corresponding structure-based sequence alignments.

FIGS. 21A-C show the structures of HhH motifs bound to DNA molecules in protein-DNA complexes. FIG. 21A is complexes of DNA with human DNA polymerase β (1bpy), FIG. 21B is E. coli helicase RuvA (1c7y), and FIG. 21C is the proposed structure for DNA bound to the modeled Topo V domain L.

FIGS. 22A and 22B show proposed models of Taq polymerse in the “closed” conformation (22A) and TopoTaq (22B) with the DNA substrate.

FIG. 23 shows the sequence similarity of TopoTaq chimera and Taq polymerase.

FIGS. 24A-B show a comparison between the amino acid sequence (SEQ ID NO. 7) of the DNA topoisomerase V from Methanopyrus TAG11 and the amino acid sequence (SEQ ID NO. 9) of the DNA topoisomerase V from Methanopyrus kandleri top5.

FIGS. 25A-G show a comparison between the nucleic acid sequence (SEQ ID NO. 8) of the DNA topoisomerase V from Methanopyrus TAG11 and nucleic acid sequence (SEQ ID NO. 10) of the DNA topoisomerase V from Methanopyrus kandleri top5.

FIG. 26 is a plot of the dependencies of the initial rates of primer extension reactions on the reaction temperature for enzymes with Taq polymerase catalytic domain.

FIG. 27 is a plot of the dependencies of the initial rates of primer extension reactions on the reaction temperature for enzymes with Pfu polymerase catalytic domain.

FIG. 28 is a plot of the dependencies of the initial rates of primer extension reactions on the reaction temperature for enzymes with Taq polymerase catalytic domain.

FIG. 29 is a plot of the dependencies of the initial rates of primer extension reactions on the reaction temperature for enzymes with Pfu polymerase catalytic domain.

FIG. 30 is a plot of the dependencies of the initial rates of primer extension reactions on the reaction temperature for enzymes with Taq polymerase catalytic domain.

FIG. 31 is a plot of the dependencies of the initial rates of primer extension reactions on the reaction temperature for enzymes with Pfu polymerase catalytic domain.

FIG. 32 is a gel image after amplification of specific DNA sequences cloned into plasmid pet21d by TaqTopoC2 DNA polymerase.

FIG. 33 is a gel image after amplification of specific DNA sequences cloned into plasmid pet21d by Pfu-C2 DNA polymerase.

FIG. 34 is an image of a gel after amplification of 110 nt region of ssDNA M13mp18(+) with ALF M13 Universal fluorescent primer and primer caggaaacagctatgacc (M13 reverse) in the presence of 0.25 M NaCl.

FIG. 35 is a gel image after amplification of DNA cloned into plasmid pet21d by TaqTopoC2 DNA polymerase in the presence of NaCl.

FIG. 36 is an image of a gel after amplification of DNA cloned into plasmid pet21d by Pfu-C2 DNA polymerase in salts.

FIG. 37 is a gel image after amplification of DNA cloned into plasmid pet21d by TaqTopoC2 and PfuC2 DNA polymerases in the presence of SYBR® Gold Nucleic Acid Gel Stain.

FIG. 38 is a gel image after amplification of DNA cloned into plasmid pet21d by TaqTopoC2 and PfuC2 DNA polymerases in the presence of SYBR® Green I Nucleic Acid Gel Stain.

FIG. 39 is a gel image after amplification of G-C-rich regions of genomic DNA by TaqTopoC2 DNA polymerase.

FIG. 40 is a gel image after amplification of plasmid DNA from E. coli bacterial culture by TaqTopoC2 DNA polymerase.

FIG. 41 is a gel image after amplification of DNA cloned into plasmid pet21d by TaqTopoC2 DNA polymerase.

FIG. 42 is a gel image after amplification of a 110 nt region of ssDNA M13mp18(+) with ALF M13 Universal fluorescent primer and primer caggaaacagctatgacc (M13 reverse).

FIG. 43 is a urea gel analysis picture for M13mp18(+) sequencing reactions with BDT v2 Kit, M13 Forward primer after 50 cycles.

FIG. 44 is a graph of fluorescence versus NaCl concentration for M13mp18(+) sequencing reactions with BDT v2 Kit, M13 Forward primer after 50 cycles.

### DETAILED DESCRIPTION OF THE PREFERRED EMBODIMENT

The present invention provides a method of transferring the enzymatic properties of HhH domains of proteins to DNA processing enzymes. In one embodiment, this invention demonstrates that the unique enzymatic properties of DNA topoisomerase V, such as its ability to work in high salt concentrations and its high processivity, can be transferred to other DNA processing enzymes. For example, this invention demonstrates that, if properly positioned, HhH repeats derived from Topo V not only restore the processivity of the Stoffel fragment of Taq polymerase to the level of Taq polymerase and restore the processivity of Pfu polymerase or fragments thereof to the level of Pfu polymerase, but also confer processivity on hybrid Taq and Pfu polymerases in high salts where the Taq and Pfu polymerases work distributively and/or are inhibited.

In one embodiment, the chimeric proteins of this invention comprise the Stoffel fragment of Taq DNA polymerase fused to an amino acid sequence having one or several HhH motifs that are not present in the corresponding wild-type polymerase. The HhH motifs either modify an existing processivity factor binding domain or introduce a new processivity factor binding domain into the DNA polymerase. That is, the processivity factor binding domain cannot be found in nature associated with the DNA polymerase domain. Thus, such a chimeric DNA polymerase is a man-made object and is not found in nature as a wild-type polymerase. The region to be inserted as described herein is usually not naturally occurring in the enzyme in which it is inserted but is taken from an enzyme in which it naturally occurs.

In another embodiment, the chimeric proteins of this invention comprise the Pfu DNA polymerase fused to an amino acid sequence having one or several HhH motifs that is not present in the corresponding wild-type polymerase. The HhH motifs either modify an existing processivity factor binding domain or introduce a new processivity factor binding domain into the Pfu DNA polymerase.

In another embodiment, the chimeric proteins comprise one or more HhH subdomains derived from Topo V and linked to either the NH₂- or the COOH-terminus of the Stoffel fragment of Taq DNA polymerase. In yet another embodiment, the chimeric proteins comprise one or more HhH subdomains derived from Topo V and linked to the COOH-terminus of Pfu DNA polymerase.

Design, Expression, and Purification of Protein Chimeras

The 5′ to 3′ exonuclease domain of Taq DNA polymerase is a structurally and functionally separate unit [15]. Its removal produces active DNA polymerases, the Stoffel fragment and KlenTaq variants with enhanced thermostability and higher fidelity but with low processivity [16, 17].

DNA Topoisomerase V from M. kandleri is an extremely thermophilic enzyme whose ability to bind DNA is preserved at very high ionic strengths [7]. An explicit domain structure, with multiple C-terminal HhH repeats is responsible for DNA binding properties of the enzyme at high salt concentrations [9, 10]. Thus, if the inhibition of Taq DNA polymerase, which has only one HhH motif, or its active derivatives (which lack the HhH motif) by salts is due to the inability of these enzymes to bind DNA, the transfer of HhH domain(s) derived from Topo V to Taq polymerase catalytic domain would restore the DNA polymerase at high salt concentrations.

In one embodiment, the chimeric DNA polymerase has a DNA polymerase domain that is thermophilic, e.g., is the DNA polymerase domain present in a thermophilic DNA polymerase, such as one from the DNA polymerase in Thermus aquaticus, Thermus thermophilus, Pfu DNA polymerase, Vent DNA polymerase, or Bacillus sterothermophilus DNA polymerase. The amino acid sequence comprising one or more HhH domains, when bound to the DNA polymerase, causes an increase in the processivity of the chimeric DNA polymerase.

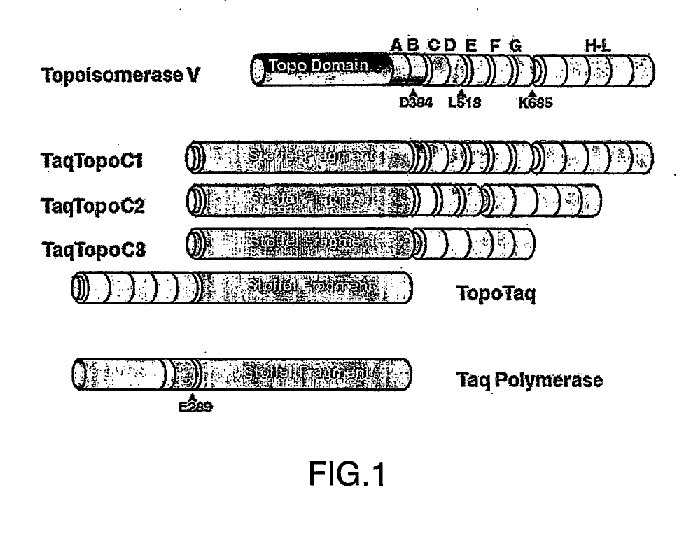

Referring to FIG. 1, five protein chimeras (also referred to herein as “hybrid proteins,” “hybrid enzymes,” or “chimeric constructs”) containing either the Stoffel fragment of Taq DNA polymerase or whole size Pfu polymerase and a different number of HhH motifs derived from Topo V were designed. The composition of polymerase and HhH domains and their positions in the chimeras as shown in FIG. 1 are: (1) TopoTaq, containing the HhH repeats H-L of Topo V (10 HhH motifs) linked to the N-terminus of the Stoffel fragment; (2) TaqTopoC1 comprising the Topo V repeats B-L (21 HhH motifs) linked to the C-terminus of the Stoffel fragment, (3) TaqTopoC2 comprising the Topo V repeats E-L (16 HhH motifs) linked to the C-terminus of the Stoffel fragment, (4) TaqTopoC3 comprising the Topo V repeats H-L (10 HhH motifs) linked to the C-terminus of the Stoffel fragment, and (5) and PfuC2 comprising repeats E-L at the C-terminus of the Pfu polymerase. Repeats are designated as in Belova et al. [9]. Repeats H-L (also known as Topo34) and F-L with a half of the repeat E are dispensable for the topoisomerase activity of Topo V [10].

The X-ray structure of Topo V and its HhH repeats is unknown, and it was assumed that H-L repeats in TopoTaq and TaqTopoC3 exist as well-defined structural domains. The overall structures of these domains are likely the same as in native Topo V, since the domains are resistant to proteolysis both in Topo V and when expressed separately (Topo 34; [10]). Also, it was thought that all Topo V domains have high internal stability in order to be functional at extremely high temperatures.

The chimeras were expressed in E. coli BL21 pLysS and purified using a simple two-step procedure. The purification procedure takes advantage of the extreme thermal stability of recombinant proteins that allows the lysates to be heated and about 90% of E. coli proteins to be removed by centrifugation. The second step involves a heparin-sepharose chromatography. Due to the high affinity of Topo V′s HhH repeats to heparin [7], the chimeras elute from a heparin column around 1.25 M NaCl to give nearly homogeneous protein preparations (>95% purity). All expressed constructs possessed high DNA polymerase activity that was comparable to that of commercial Taq DNA polymerase.

In one embodiment, the chimeric proteins of this invention may comprise a DNA polymerase fragment linked directly end-to-end to the HhH domain. Chemical means of joining the two domains are described, e.g., in Bioconjugate Techniques, Hermanson, Ed., Academic Press (1996), which is incorporated herein by reference. These include, for example, derivitization for the purpose of linking the moieties to each other by methods well known in the art of protein chemistry, such as the use of coupling reagents. The means of linking the two domains may also comprise a peptidyl bond formed between moieties that are separately synthesized by standard peptide synthesis chemistry or recombinant means. The chimeric protein itself can also be produced using chemical methods to synthesize an amino acid sequence in whole or in part, e.g., using solid phase techniques such as the Merrifield solid phase synthesis method.

Alternatively, the DNA polymerase fragment can be linked indirectly via an intervening linker such as an amino acid or peptide linker. The linking group can be a chemical crosslinking agent, including, for example, succinimidyl-(N-maleimidomethyl)-cyclohexane-1-carboxylate (SMCC). The linking group can also be an additional amino acid sequence. Other chemical linkers include carbohydrate linkers, lipid linkers, fatty acid linkers, polyether linkers, e.g. PEG, etc. The linker moiety may be designed or selected empirically to permit the independent interaction of each component DNA-binding domain with DNA without steric interference. A linker may also be selected or designed so as to impose specific spacing and orientation on the DNA-binding domains. The linker may be derived from endogenous flanking peptide sequence of the component domains or may comprise one or more heterologous amino acids. Linkers may be designed by modeling or identified by experimental trial.

As demonstrated in the discussion and examples provided below, this invention also provides methods of amplifying a nucleic acid by thermal cycling such as in a polymerase chain reaction (PCR) or in DNA sequencing. The methods include combining the nucleic acid with a chimeric DNA polymerase having a DNA polymerase linked to an amino acid sequence comprising one or more helix-hairpin-helix (HhH) motifs not naturally associated with said DNA polymerase, wherein said amino acid sequence is derived from Topoisomerase V. The nucleic acid and said chimeric DNA polymerase are combined in an amplification reaction mixture under conditions that allow for amplification of the nucleic acid. Such methods are well known to those skilled in the art and need not be described in further detail.

HhH Domains Confer DNA Polymerase Activity on Chimeras in High Salts

The polymerase activities of the four chimeras were tested by measuring initial rates of primer extension reactions. The reactions were carried out at low concentrations of substrate, when the initial rates were proportional both to total protein and PTJ concentrations. When [PTJ] is much less than Kmₐₚₚ, the initial rate is determined as in Equation 1:
v₁=kₐₚₚ/Kmₐₚₚ*[Eₜ]*[PTJ]₁ Eq. 1
where Kmₐₚₚ and kₐₚₚ are apparent Michaelis and catalytic constants, respectively.

The concentrations of sodium chloride (NaCl), potassium chloride (KCl) and potassium glutamate (KGlu) were varied to assess inhibition of the Stoffel fragment and KlenTaq, and the four chimeras by salts, and to estimate the effects of the HhH domains.

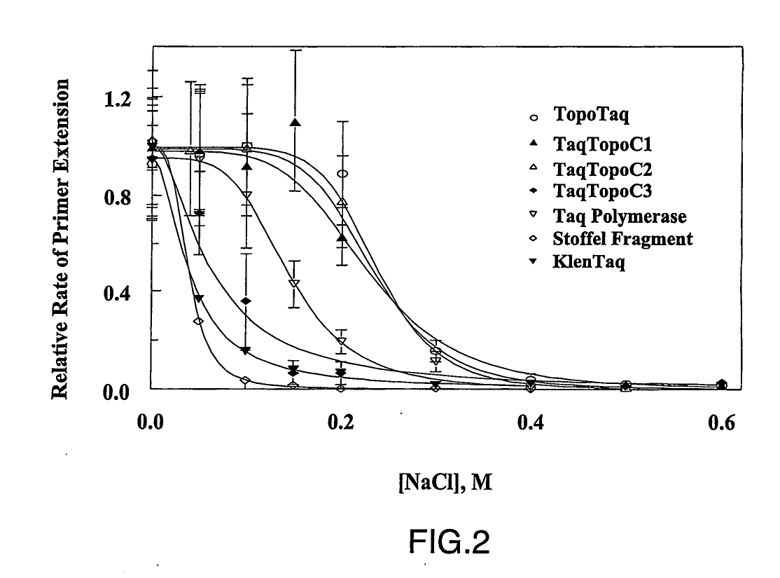

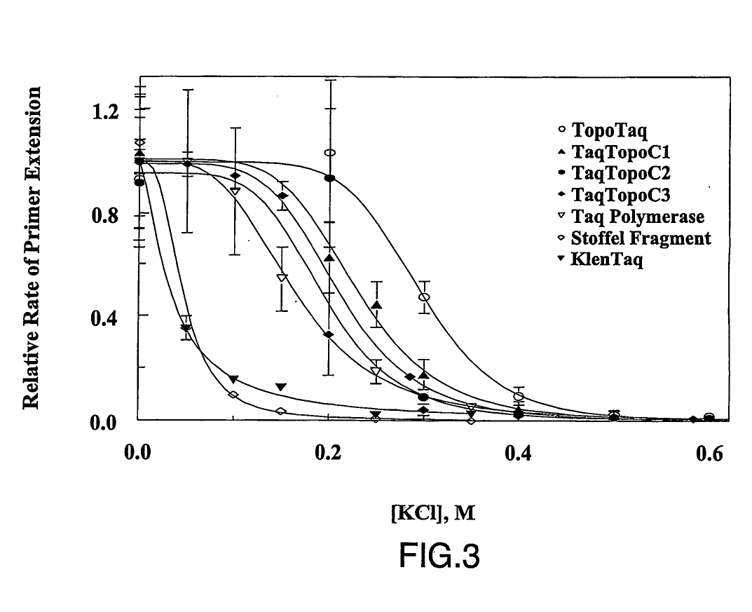

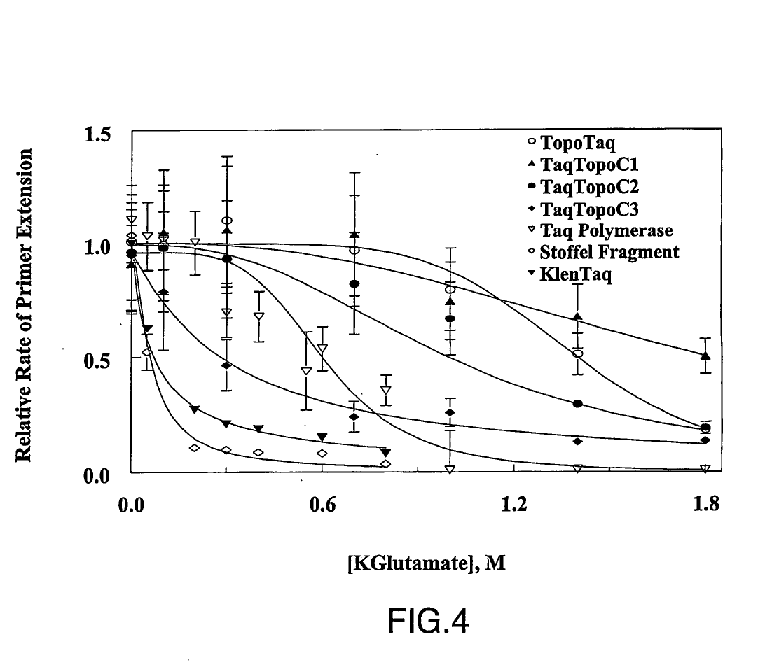

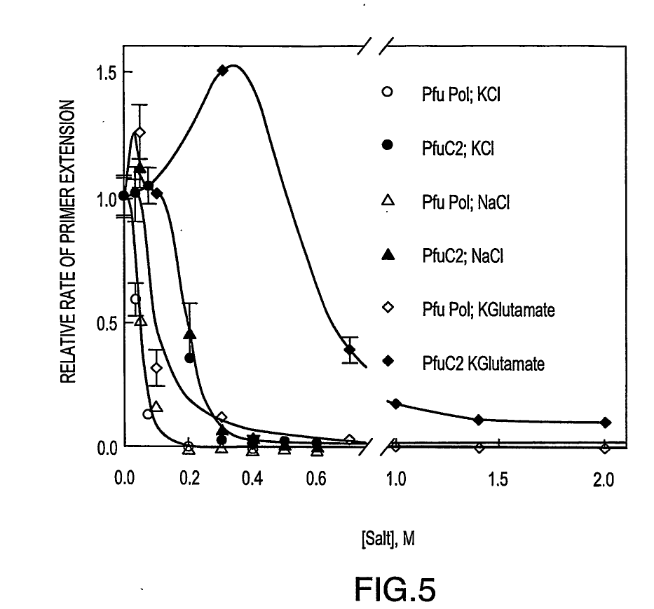

FIGS. 2, 3, 4 and 5 show the activities of Taq DNA polymerase, Taq DNA polymerase fragments (Stoffel fragment and KlenTaq), the four Taq-Topo V chimeras, and Pfu and PfuC2 polymerases in salts. Initial rates of primer extension reactions for the proteins were determined as described in Example 3. The dependencies of the rates on salt concentrations were plotted for NaCl (FIGS. 2 and 5), KCl (FIGS. 3 and 5), and KGlu (FIGS. 4 and 5). The results presented in FIGS. 2-5 show sigmoid curves, indicating the cooperative inhibition of the enzymes by these salts. Experimental values of initial polymerization rates were analyzed by nonlinear regression analysis using Equation 2:

$$v = \frac{{v}_{o}}{1 + {( \frac{[ Salt ]}{{K}_{i}} )}^{α}} \qquad Eq . 2$$

where v and v₀ are initial primer extension rates with and without salt, respectively, Kᵢ is the apparent inhibition constant; and α is the cooperativity parameter. The values for Kᵢ and α are listed in Table 1.

In Table 1, to take into account the activation of Pfu polymerase and the PfuC2 hybrid by KGlu (data entries marked with an asterisk (*), the experimental values of initial polymerization rates were analyzed by nonlinear regression using the Equation 3:

$$v = \frac{{v}_{o} \cdot ( 1 + β \cdot {[ Salt ]}^{y}}{1 + {( \frac{[ Salt ]}{{K}_{i}} )}^{α}} \qquad Eq . 3$$

where v and v₀ are initial primer extension rates with and without salt, respectively; Kᵢ is an apparent inhibition constant, α is a parameter of cooperativity, β and γ are parameters of activation. Since γ≅2, it is likely that two glutamate ions bind to the Pfu polymerase catalytic domain without inhibiting the polymerase activity.

TABLE 1
Parameters of inhibition of Taq DNA polymerase and
TopoTaq chimeras by salts

| | NaCl | | KCl | | KGlu | |
|---|---|---|---|---|---|---|
| Protein | Kᵢ | α | Kᵢ | α | Kᵢ | α |
| TopoTaq | 241.3 ± 14 | 7.04 ± 1.4 | 291.1 ± 10 | 6.45 ± 0.6 | 1403.0 ± 20 | 6.03 ± 0.4 |
| TaqTopoC1 | 228.4 ± 6 | 4.27 ± 0.2 | 231.2 ± 12 | 5.02 ± 0.6 | 1730.0 ± 125 | 2.45 ± 0.6 |
| TaqTopC2 | 238.4 ± 3 | 6.77 ± 0.2 | 251.0 ± 6 | 8.97 ± 0.6 | 1164.5 ± 42 | 4.34 ± 0.5 |
| TaqTopC3 | 69.0 ± 14 | 1.86 ± 0.2 | 187.7 ± 2 | 3.87 ± 0.1 | 295.8 ± 92 | 1.21 ± 0.2 |
| Taq | 138.7 ± 6 | 3.24 ± 0.5 | 161.0 ± 6 | 3.50 ± 0.2 | 610 ± 51 | 4.45 ± 0.3 |
| Polymerase | | | | | | |
| Stoffel | 38.6 ± 3 | 3.45 ± 0.2 | 45.8 ± 4 | 2.92 ± 0.1 | 59.6 ± 38 | 1.47 ± 0.4 |
| Fragment | | | | | | |
| KlenTaq | 40.0 ± 5 | 1.83 ± 0.1 | 32.7 ± 7 | 1.49 ± 0.2 | 71.0 ± 24 | 0.89 ± 0.1 |
| Pfu | 51.5 ± 1 | 2.39 ± 0.1 | 42.6 ± 1 | 3.65 ± 0.1 | 42.8* ± 6 | 3.24 ± 0.2 |
| polymerase | | | | | | |
| PfuC2 | 159.6 ± 33 | 3.62 ± 0.8 | 176.8 ± 3 | 4.68 ± 0.1 | 424.8* ± 9 | 5.76* ± 0.2 |

The dependencies of primer extension rates on salt concentrations as shown in FIGS. 2-5 can be explained if one considers α as the number of ions, bound either to the protein or to DNA, that prevent formation of a productive polymerase-DNA complex. However, the results presented below suggest that anions play a major role in the inhibition.

For Taq polymerase, inhibition constants (Kᵢ) for NaCl and KCl are essentially the same, yet substituting KCl with KGlu increases the Kᵢ 4-fold (Table 1). Hence, Taq polymerase is sensitive to anions. The cooperativity parameter α was very similar for all salts tested and suggests that as many as four anions bound simultaneously to the protein are involved.

The Stoffel and KlenTaq fragments of Taq DNA polymerase have almost equal sensitivities to chloride ions, which is about four times higher than the sensitivity of Taq polymerase to chloride ions. Potassium glutamate inhibited these fragments only about 1.5 to 2 times less efficiently than NaCl or KCl, implying that the HhH domain can be responsible for the resistance of Taq polymerase to glutamate ions. It was observed that KlenTaq had consistently lower values of the cooperativity parameter α than the Stoffel fragment, suggesting that the additional N-terminal amino acids could mask some anion-binging sites on the catalytic domain.

As shown in Table 1, TopoTaq has higher inhibition constants (Kᵢ) in salts as compared with Taq polymerase, and may require six to seven anions to be bound for inhibition. As a result, TopoTaq is active at much higher salt concentrations than Taq DNA polymerase. For example, a 20% inhibition of primer extension reaction occurs at about 200 mM NaCl for TopoTaq versus about 90 mM NaCl for Taq DNA polymerase. The TopoTaq chimera also displays little distinction between sodium and potassium cations and is less sensitive to glutamate anions versus chloride anions (FIGS. 2-4).

It was observed that the 21 and 16 HhH motifs at the COOH terminus of the Stoffel fragment in TaqTopoC1 and TaqTopoC2, respectively, also increase the polymerase activities of chimeras in the presence of salts. For example, 20% inhibition occurred at about 160 mM NaCl for TaqTopoC1 and at about 195 mM NaCl for TaqTopoC2 (FIG. 2). Similar to Taq polymerase, the TaqTopoC1 and TaqTopoC2 chimeras show no difference in inhibition by KCl versus NaCl (with the cooperativity parameter α about equal to 5), and glutamate anions were much more preferable than chloride anions. However, the cooperativity parameter for the TaqTopoC1 and TaqTopoC2 chimeras in the case of glutamate is lower compared to that of Taq polymerase or TopoTaq, suggesting that only two glutamate ions are involved in the rate inhibition.

TaqTopoC3 behaves differently in salts than TaqTopoC1 and TaqTopoC2. Although inhibition of TaqTopoC3 by KCl is similar to that of TaqTopoC1 or TaqTopoC2 (with a≈5, but with a slightly lower Kᵢ similar to that of Taq DNA polymerase), replacement of potassium ions by sodium ions results in a much stronger inhibition of the TaqTopoC3 polymerase activity and, at the same time, decreases the number of inhibiting ions to about 2. Consequently, just 30 mM NaCl inhibits the enzyme by 20%. TaqTopoC3 has about a fivefold relative decrease in sensitivity to KGlu with respect to NaCl (but not to KCl), which is similar to other hybrids. However, in case of glutamate no cooperativity at all was found, suggesting that only one glutamate ion per molecule is involved in the inhibition of TaqTopoC3.

With the exception of TaqTopoC3, introduction of C-terminal domains of Topo V into the hybrid proteins significantly extends the range of salt concentrations for the polymerase activity. This effect is due to the increase of both Kᵢ and α, allowing chimeras to maintain their full activity at high salt concentrations. Such behavior indicates that in the catalytic domain the incorporation of deoxynucleotides is not affected even at high concentrations of salts. Rather, binding of DNA substrate is impaired, due to competition between DNA and the anions for the positively charged amino acid residues of the proteins. Raising the number of HhH motifs from 11 to 23 at the COOH-terminus of the Stoffel fragment made the hybrid enzymes progressively more resistant to salts. TopoTaq had the highest resistance to chloride-containing salts.

It was observed that while both TopoTaq and TaqTopoC3 contain the same C3 HhH domains (FIG. 1), TaqTopoC3 did not show a significant increase in salt resistance. While not wishing to be bound by any particular theory, one explanation of this difference may be that when the C3 domain is linked to the NH₂-terminus of the Stoffel fragment, as in the case of TopoTaq (FIG. 1), the C3 domain may interact specifically with double-stranded DNA or primer-template regions (e.g., encircle or wrap double-stranded regions [9]). In contrast, when the C3 domain linked to the COOH-terminus of the Stoffel fragment, as in the case of TaqTopoC3, the C3 domain is remote from DNA.

In the TaqTopoC1 and TaqTopoC2 constructs, both C1 and C1 domains include the C3 domain, but they also contain additional HhH repeats, which form rather loose structures as shown by limited proteolysis of Topo V [10]. These additional repeats may extend the “HhH arm” sufficiently far to reach a DNA template (FIG. 1). The sensitivity of Pfu DNA polymerase to salts was almost identical to that of Stoffel or KlenTaq fragments of DNA polymerase from Thermus aquaticus, possibly indicating the close functional similarity of charged amino acid residues in the active sites of these enzymes from different structural families. Attachment of Topo V HhH domains to C-terminus of Pfu polB significantly increased the resistance of polymerase activity to salts (FIG. 5). Both Pfu DNA polymerase and the chimera PfuC2 demonstrated virtually indistinguishable curves for KCl versus NaCl, suggesting no role for cations in inhibition. Interaction of both Pfu polymerase and PfuC2 with KGlu was more complicated because of activation at low concentrations of glutamate anions, as was also observed for other archaeal polII DNA polymerases (data not published). However, the Topo V domains greatly increased the resistance of Pfu polII activity to high levels of KGlu.

Activation of TopoTaq in Betaine

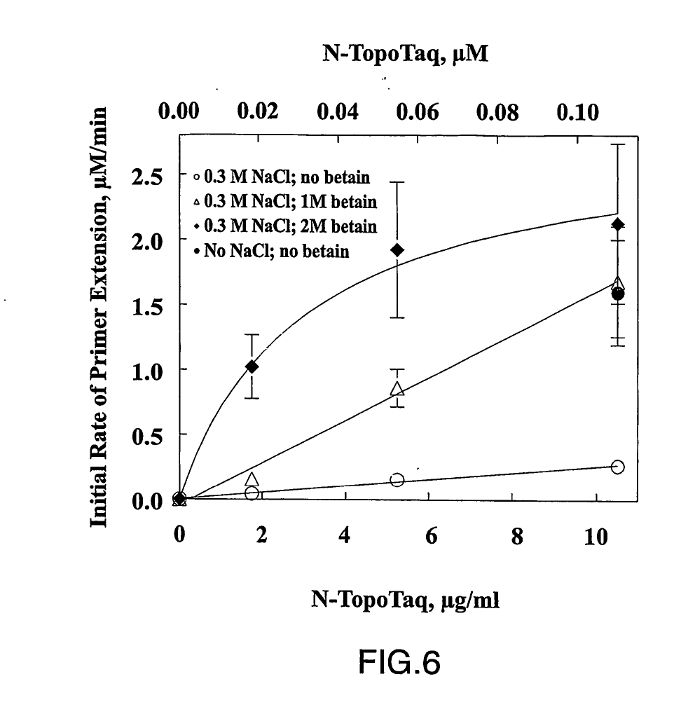

Betaine at 1 M significantly increased the initial rate of a primer extension reaction catalyzed by TopoTaq in the presence of 0.3 M NaCl. Further increases of betaine concentrations up to 2 M at 10.5 μg/mL TopoTaq did not affect the primer extension activity. Higher betaine concentrations inhibited the polymerase reaction (data not shown). The activation by betaine only occurred if TopoTaq was inhibited by NaCl and brought the polymerase activity back to the level of the non-inhibited enzymes. The results, which are shown in FIG. 6, are consistent with the increased affinity of the enzyme to a primer-template junction (PTJ) substrate in the presence of betaine. The data also suggest that betaine enhances the rate of primer extension (Vₘₐₓ) about 6-fold in the active site of the construct.

The reason that betaine activates TopoTaq in 0.3 M NaCl is of some interest. Since binding DNA to proteins involves both electrostatic and non-electrostatic interactions, it seems possible to eliminate the electrostatic interactions at high salt concentrations. It had been observed previously that the very slow disassociation of Topo V from DNA at a low salt concentration (50 nM NaCl) prevents cycling of the enzyme between DNA molecules [7]. However, at 0.3 M NaCl, the binding becomes less tight, so that Topo V is able to cycle between DNA molecules. Accordingly, the activity of Topo V on double-stranded DNA substrates is strongly inhibited by single-stranded DNA added at 0.3 M NaCl [18]. Such behavior of Topo V suggests that the charge interactions are predominant in the binding of DNA to HhH domains. However, this non-specific inhibition by dsDNA could be abolished if 2.2 M betaine is added to 0.3 M NaCl, resulting in selective binding of Topo V to dsDNA.

The inhibition of DNA synthesis by anions presumes the combined effect of salts on electrostatic interaction of DNA substrate with both Taq catalytic domain and HhH structures of Topo V. The lower activity of TopoTaq in 0.3 M NaCl alone, as compared with that in the presence of 0.3 M NaCl and 1-2 M betaine, can be attributed to binding of the single-stranded region of PTJ by the HhH domains, which facilitates dissociation of the substrate DNA from the catalytic domain. In the presence of betaine, the TopoTaq HhH domains may preferentially bind the double-stranded region of the duplex and provide proper orientation of PTJ with respect to the polymerase catalytic site.

Rees et al. [19] found that both the electrostatic properties of DNA and the β-conformation of double-stranded DNA were retained even at very high concentrations of betaine (about 5.5 M). They observed that, in the absence of salts, betaine did not have much of an affect the preferential interaction of RNAse A with a single-stranded DNA and, even at very high concentrations, betaine did not disrupt the electrostatic interactions of DNA with the protein. However, their results also indicate that at very high concentrations of betaine RNAse A starts binding primarily double-stranded DNA.

The results disclosed in the present invention support earlier conclusions that prevention of non-specific protein-nucleic acids interactions might be one of unaccounted physiological roles of zwitterionic compounds like betaine or other modified tetraalkylammonium compounds [18-20].

Dependence of Primer Extension Rate on Substrate Concentration

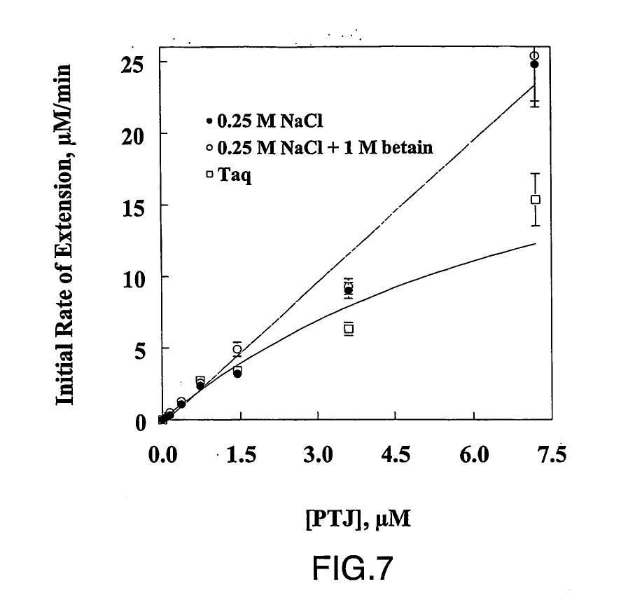

As stated above, the affinity of DNA polymerases to various DNA substrates has not been clearly defined in many cases. The initial rates of primer extension reactions were thus measured using a wide range of PTJ concentrations to assess the affinity of the hybrid enzyme TopoTaq to DNA performed as described in Example 3 with varying concentrations of PTJ. FIG. 7 illustrates the dependencies of initial primer extension rates on the concentration of PTJ for reactions catalyzed by TaqDNA polymerase, TopoTaq in the presence of NaCl, and TopoTaq in the presence of NaCl and betaine.

FIG. 7 shows that the initial rates of primer extension by Taq DNA polymerase and TopoTaq in the presence of 0.25M NaCl were almost proportional to the substrate concentration up to 7.2 μM PTJ. This could indicate that apparent Km's for these reactions are higher than 10⁻⁶ M.

Role of HhH Domains in Processive DNA Synthesis

For DNA replication in vivo and for many in vitro applications, it is important to carry out processive synthesis of DNA. That is, DNA polymerases need to perform a sequence of polymerization steps without intervening dissociation from the growing DNA chains. This property, however, strongly depends on the nature of the polymerase, the sequence of the DNA, and additional reaction conditions such as salt concentration, temperature or presence of specific proteins. Processivity can be described as a quantitative measure of the ability of an enzyme to carry out stepwise catalytic reactions while being attached to a polymeric substrate. For example, processivity can refer to the probability that the polymerase will incorporate the next nucleotide rather than dissociate from a primer-template junction.

von Hippel et al. introduced a model to describe quantitatively the processive synthesis of DNA and defined the “microscopic processivity parameter,” pᵢ, which is the probability that a polymerase positioned at the PTJ at template position i will not dissociated from the DNA in translocation to position i+1 [21]. This parameter can be determined experimentally by measuring the fraction of extended primers that reach position n, but do not terminate there under single-hit conditions of the assay, that is, conditions where a DNA polymerase dissociating from the PTJ does not have a chance to bind to the extended products [14, 21, 22]. To characterize processive DNA synthesis on heterogeneous templates, the “geometric mean microscopic processivity parameter” was introduced [21]. This parameter is particularly useful for comparison of the same number of nucleotide attachments in different reactions. However, any premature termination of extension within the defined length of DNA synthesis (as in case of primer extensions in salts) renders this parameter zero.

To bypass this limitation, the “processivity equivalence parameter,” Pₑₕ is defined herein. Pₑₕ is a value of processivity of a DNA polymerase reaction for an infinite homopolymer substrate that would produce the same average substrate extension per polymerase binding event as a reaction with a heterogeneous sequence characterized by non-constant values of pᵢ.

It has been shown theoretically [13] that the probability of producing a primer extended by exactly n residues could be written using the microscopic processivity parameter

$${p}_{i} as \text{:} {P}_{n} = {\sum }_{i = 0}^{n - 1} ( 1 - {p}_{n} ) \prod {p}_{i} .$$

Therefore, the average length of extension (Lₐᵥ) would be equal to

$${\sum }_{n = 1} {n}^{*} {P}_{n} .$$

As for short extensions (at increased salt concentrations), the microscopic processivity parameters decrease to zero within experimental error, and the average length of extension per polymerase binding event can be calculated directly from experimental microscopic processivity data using Equation 4:

$$lav = {\sum }_{n = 1}^{{n}_{max}} {n}^{*} {P}_{n} \qquad Eq . 4$$

where nₘₐₓ is the maximum number of nucleotide attachments allowed by a template. However, for highly processive synthesis, the average extension per binding event could be greater then the physical length of the template. In this case, the Lₐᵥ value was calculated using Equation 5:

$${L}_{av} = {\sum }_{n = 1}^{{n}_{max}} {n}^{*} Pn + {\sum }_{n = {n}_{max} + 1}^{∞} {n}^{*} {P}^{′} n \qquad Eq . 5$$

where P′n=(1−Pn)*Pn; and P is the geometric mean microscopic processivity parameter was calculated to produce the best approximation of the theoretical value of Lₐᵥ. (This also can be considered as average extension per binding event on an infinite hypothetical substrate that has nₘₐₓ nucleotides of the original sequence continued by a homo-polymer tail on which DNA polymerase synthesize with processivity equal to the geometric mean microscopic processivity parameter of the original sequence). Then, since for a homopolymer, Lₐᵥ=1/(1−P) [5], the homopolymer “processivity equivalence parameter” is defined as Pₑₕ=1−(1/Lₐᵥ).

The modified processivity equivalence parameter provides convenient access to the overall effects of salts on processivity in primer extensions, as it eliminates the length restriction inherent to the geometric mean microscopic processivity parameter.

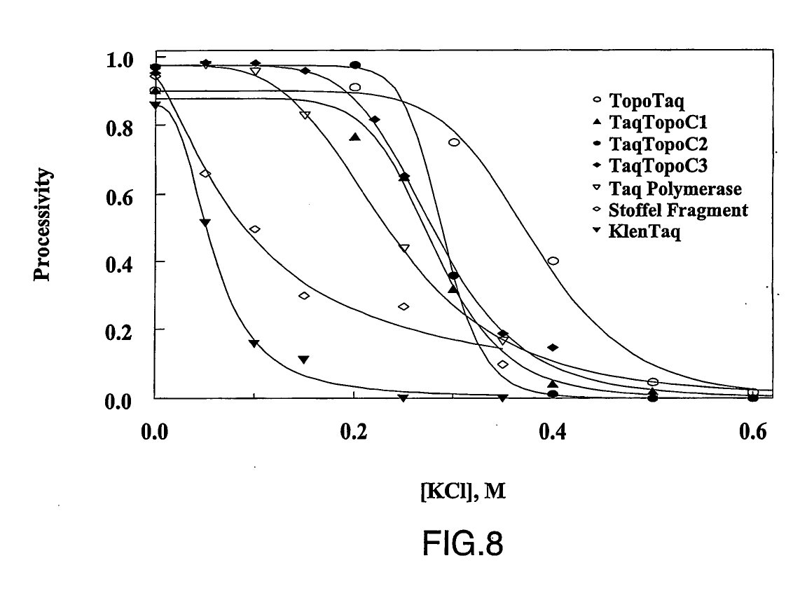

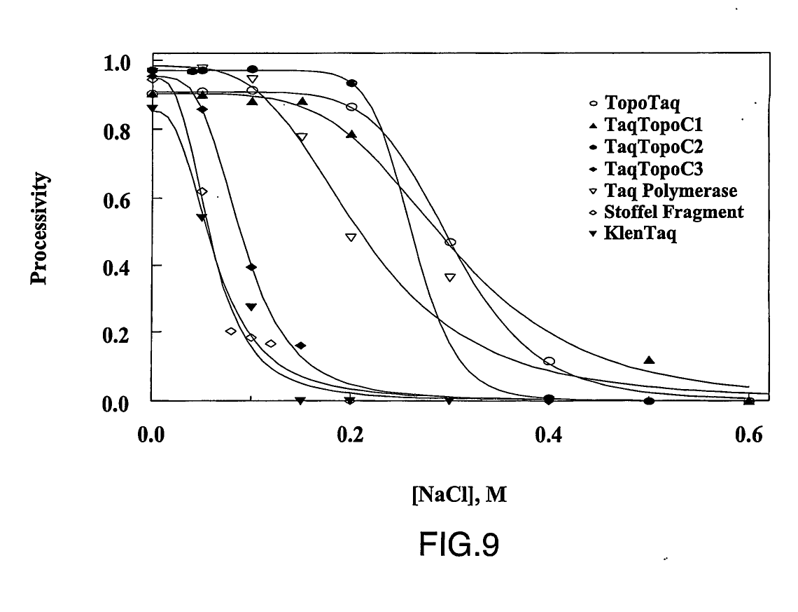

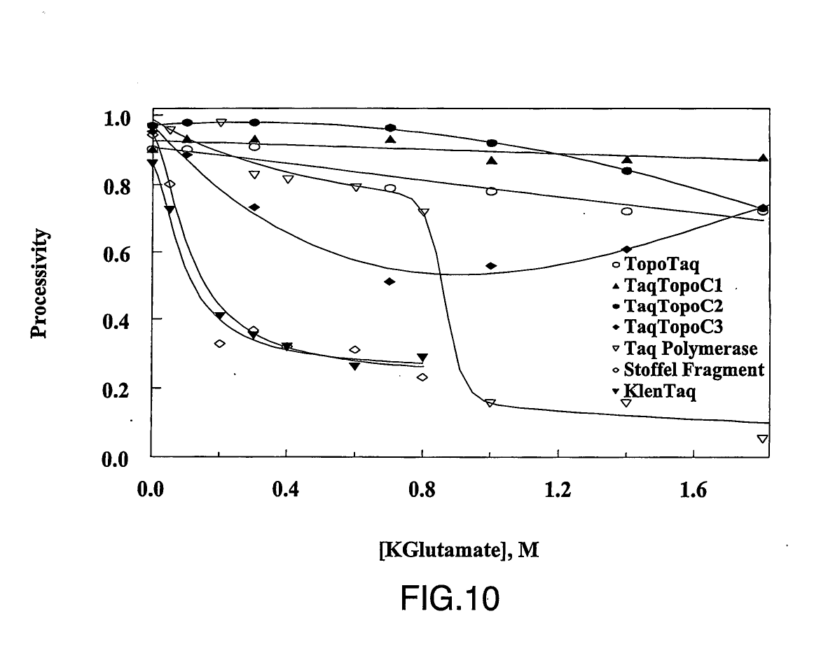

The processivities (Pₑ) of Taq DNA polymerase, Taq DNA polymerase fragments, and the Taq-Topo V chimeras in primer extension reactions in various salts were determined as described in Example 4. The dependencies of the rates on salt concentrations were plotted for NaCl (FIG. 8), KCl (FIG. 9), and KGlu (FIG. 10). The solid lines are the theoretical inhibition curves by these salts. In the absence of added salts, at 75° C., Taq DNA polymerase and its Stoffel fragment have high processivity (Pₑ=0.90-0.98). KlenTaq has the lowest value of Pₑ (0.86), suggesting that additional N-terminal amino acids (as compared to the Stoffel fragment) may interfere with proper positioning of the substrate and facilitate its dissociation from the catalytic domain of the protein. Similar effects might be responsible for the slight loss in processivity of TopoTaq, in which the HhH domains are attached to the N-terminus of the Stoffel fragment, and TaqTopoC1, having an very long fusion at the C-terminus of the Stoffel fragment (Pₑ=0.9 for both chimeras). In contrast, shorter additions to the C-terminal residue (as with TaqTopoC2 and TaqTopoC3) do not seem to affect the processivity core polymerase.

Chloride salts completely inhibit the processive synthesis of DNA (FIGS. 8 and 9). For some constructs, the processivity was more sensitive to NaCl versus KCl than the initial rates. This may indicate that dissociation of DNA substrate from the polymerases is more sensitive to sodium ions than to potassium ions as compared to rates of association. However, the apparent linear correlation between processivity and apparent rate of association (data not shown) confirm that the same anion-binding sites on the protein involved in both formation and destabilization of the productive complex are accessible to chloride ions.

In contrast, in potassium glutamate the processive synthesis by the DNA polymerases could not be eliminated, even at very low rates of synthesis (FIG. 10). Because of the functional dependence of the polymerization rate on processivity, a PTJ duplex, once bound to the polymerase, makes some inhibitory anion-binding sites inaccessible to glutamate ions but not to chloride ions. Inhibition of processivity by glutamate ions also demonstrates that cations play a major role in modification of DNA polymerase activity. The inhibition demonstrates that, at least in TopoTaq, the HhH domains support proper orientation of the PTJ substrate in polymerase active site in addition to tethering the whole protein to DNA substrate, as is likely the case with other constructs.

The curves for Taq polymerase, its fragments and the chimeric constructs (FIGS. 8-10) reveal separate roles of the catalytic polymerase domain and HhH domains in maintaining processivity. The inhibition suggests that the HhH domains most likely support proper orientation of the PTJ substrate in polymerase active site.

Without added salts, the Stoffel fragment has a processivity very close to that of Taq polymerase. Addition of N-terminal amino acids that produce KlenTaq slightly decreases the processivity, which is likely due to of interference of the attachment with the optimal PTJ position.

According to the structure of the catalytic domain of Taq polymerase [23], some anion-binding sites that stabilize the position of PTJ can easily interact with anions from solution (especially residues Arg-677, and -746 and Lys-508). However such interactions do not completely destabilize the productive polymerase-PTJ complex, because the other four basic amino acid residues (Arg-487, -587, -728 and Lys-540) in proximity to the substrate are less accessible. The HhH domain in Taq polymerase apparently stabilizes the proper position of PTJ in the protein-substrate complex. However, when the domain gets dissociated at higher salt concentrations, PTJ remains bound, as in Taq DNA polymerase fragments. Constructs TopoTaq, TaqTopoC1, TaqTopoC2, TaqTopoC3 and PfuC2 do not seem to have their HhH domains dissociated from the substrate even at very high glutamate ion concentrations. In contrast, the increase of the salt level apparently helps to organize tighter binding of HhH domains with the substrate as in the case with TopoTaqC3.

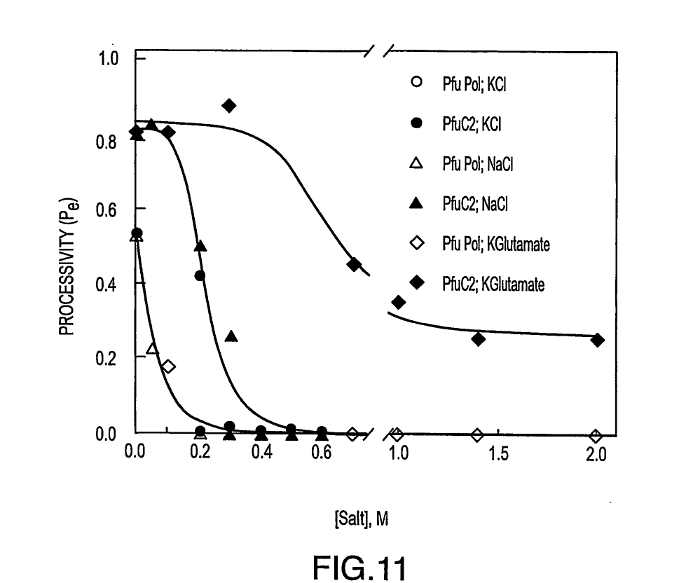

Pfu and PfuC2 DNA polymerases (FIG. 11 from PNAS) have lower values of Pₑ than Taq polymerase, its fragments, and the Taq polymerase-Topo V hybrids as it is shown in FIGS. 8-10.

It is important to point out the difference in effect of the fused HhH domains on the processivity of Taq and Pfu chimeras. The processivity of PfuC2 is considerably higher than that of the original Pfu polymerase, both without salt added and at increased salt concentrations (FIG. 11). In contrast, it appears that the processivity of Taq chimeras (FIG. 8-10) never exceeds that of the unmodified Taq polymerase (corresponding to the reported average length of extended products 20-40 nt [28]). Rather, the HhH domains raise the processivity of the chimeras in high salts to the level of the core polymerase. As the rate of nucleotide incorporation by the Taq catalytic domain is not affected by salts if the PTJ is bound to the active site (FIGS. 2, 3 and 4), the processivity is determined by the rate of dissociation of the polymerase-DNA complex. It is likely that at low salt concentrations the dissociation of Topo V HhH domains from DNA substrates occurs with similar or even lower rates as that of the Taq polymerase catalytic domain. Yet, no increase in processivity within the precision of our measurements could be found in experiments with short substrate DNA, as the processivity of the catalytic domain alone is very high. However, at high salt concentrations, the detachment of the salt-resistant Topo V domains clearly becomes the rate-limiting step for the dissociation of the entire polymerase-DNA complex. In contrast, the rate of dissociation of Pfu polymerase catalytic domains seems to be much higher than that of Topo V HhH domains and contributes to low processivity of DNA synthesis by this polymerase together with its 3′→5′ exonuclease activity. The hybrid PfuC2 has improved binding to DNA, and its lower processivity in DNA extension, as compared to Taq polymerase chimeras, can be attributed to the balance of the polymerase/exonuclease reactions in the core Pfu polB domain.

Thermostability of DNA Polymerase Chimeras

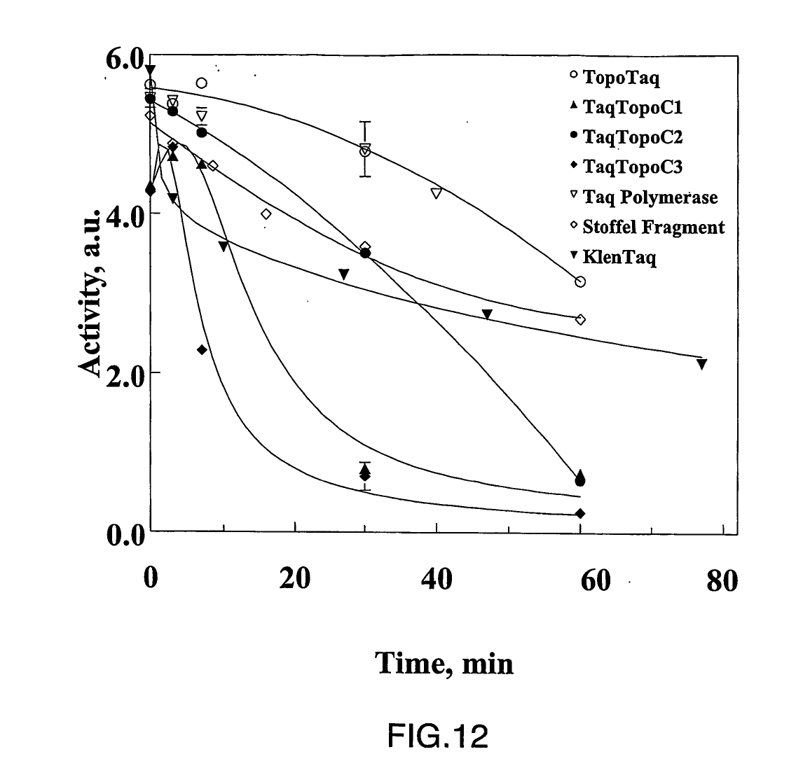

Introduction of additional protein domains into TaqDNA polymerase could have adverse effects on the enzyme stability, particularly at high temperatures [24]. The thermal stabilities of the protein chimeras, Taq polymerase, the Stoffel fragment, Pfu polymerase, and the chimeric constructs were examined by measuring their activities in a primer extension assay after incubation at high temperatures in various buffers. The proteins were incubated in 0.5 M NaCl at concentration 0.1 mg/mL and their activity was determined as described in Example 5. The results are shown in FIGS. 12-15.

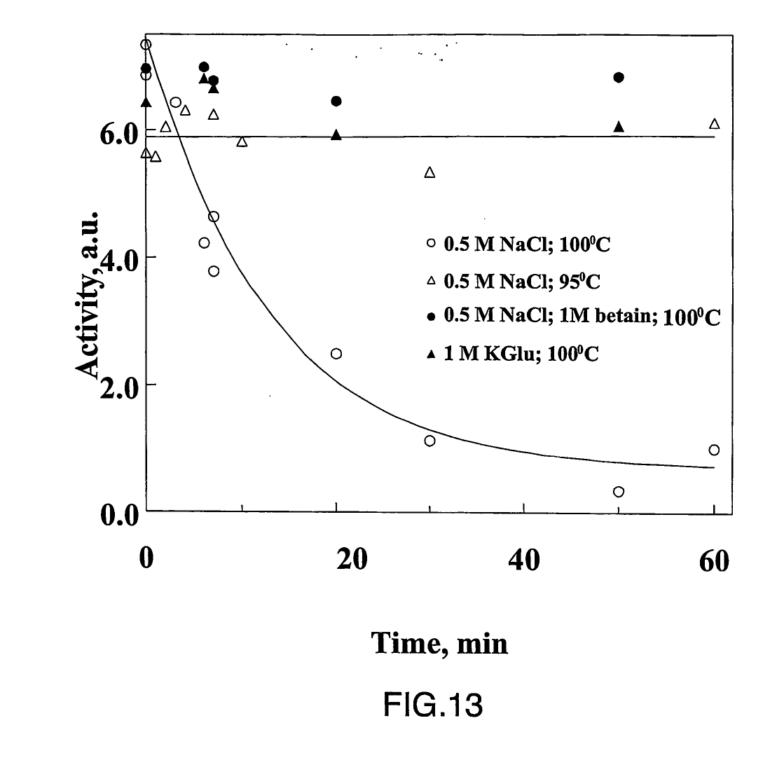

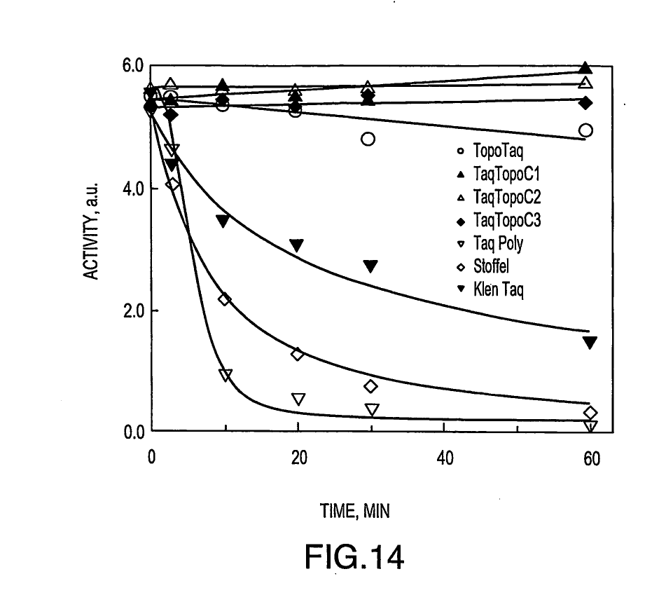

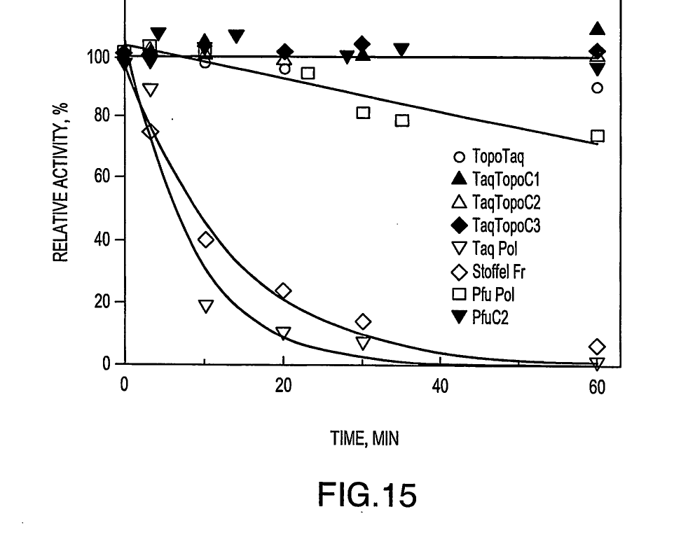

FIG. 12 shows thermal inactivation curves for Taq polymerase, the Stoffel and KlenTaq fragments, and the protein chimeras in 0.5 M NaCl at 95° C. FIG. 13 displays the thermoinactivation curves of 0.1 mg/mL TopoTaq chimera at 100° C. in various media. FIG. 14 shows thermal inactivation curves for Taq polymerase, the Stoffel and KlenTaq fragments, and the protein chimeras in 1 M potassium glutamate and 1 M betaine at 100° C. FIG. 15 shows the thermal inactivation cures for Pfu and PfuC2, with other enzymes and constructs used as internal controls.

The results shown in FIG. 12 demonstrate that all proteins, except for the Stoffel fragment, display at least biphasic inactivation in the presence of 0.5 M NaCl. This suggests that HhH domains attached to the core polymerase fragment affect the stability of the resulting hybrid proteins.

TaqDNA polymerase appears to be more stable at 95° C. than was previously reported [16], probably because of stabilization by NaCl. The TaqTopoC1, TaqTopoC2 and TaqTopoC3 chimeras, in which the HhH domains of Topo V are attached to COOH-terminus of the Stoffel fragment, have reduced thermostability compared to Taq polymerase. TaqTopoC2 is the most stable of these three hybrids and behaves in a manner similar to TopoTaq or TaqDNA polymerase. As shown in FIG. 12, all three of these chimeras produced upward curved plots, suggesting a prolonged initial stabilization of the active proteins by the added domains, followed by decreasing stability at longer incubation times.

The plot for TaqTopoC1 is similar to that of TaqTopoC3 but does not resemble that of TaqDNA polymerase. The TaqTopoC1 plot shows a brief increase of activity following by a relatively fast inactivation. This demonstrates that at certain positions the HhH domains can bring about a dramatic destabilization of the constructs. As a result, both chimeras with C1 and C3 domains were very unstable at 95° C.

At 0.5 M NaCl, the TopoTaq hybrid is at least as stable as TaqDNA polymerase at 95° C. when equal concentrations of the proteins are used in experiments, and it maintains some activity even after incubation at 100° C. (FIG. 13). The thermostability of TopoTaq increases at higher concentration of the protein (0.35 mg/mL at 95° C.; FIG. 13).

It was discovered that the addition of betaine or potassium glutamate greatly increases the thermostability of all constructed chimeras with the amino acid sequences of Topo V HhH repeats attached to the polymerase domain of Taq polymerase, allowing them to stay at 100° C. for at least 1 hour without any detectable loss of activity (FIG. 14). In contrast, Taq polymerase, the Stoffel fragment, and even the highly resistant Pfu show significant inactivation (FIG. 14); therefore the effect of stabilization can be specifically attributed to the attached Topo V domains.

DNA Sequencing and PCR with TopoTaq at High Salt Concentrations

The TopoTaq chimera was used in a direct cycle sequencing experiment at higher salt concentrations. The TopoTaq chimera was able to perform both primer extension and chain termination with ddNTPs at 0.25 M NaCl, whereas unmodified TaqDNA polymerase was totally inefficient under these conditions. Since a regular sequencing protocol (developed for TaqDNA polymerase) was used [25], it was assumed that NaCl did not cause any significant changes in dNMP/ddNMP incorporation. Also, it appeared that the ability to incorporate 7-deaza-dGTP (used in this protocol) was not impaired. This provides further evidence in support of the inventors' theory that salts do not inhibit the catalysis of chain elongation by active sites of DNA polymerases, but interfere with the ability of the proteins to remain bound to DNA substrate during synthesis.

Consistently, the mutant TopoTaq chimera was constructed that was able to incorporate fluorescent dideoxynucleotide chain terminators into Sanger fragments in cycle sequencing experiments. The chimeric DNA polymerases of this invention could also perform in vitro DNA amplification in polymerase chain reactions at high salt concentrations and in the presence of high levels of intercalating dyes and organic inhibitors.

The results presented herein indicate that hybrid enzymes containing elements of several proteins can be useful in understanding how individual domains in an enzyme's structure relate to its function and what changes can be tolerated within a particular construct.

The use of fragments of proteins from extremely thermophilic organisms may enhance the probability of obtaining stable constructs that combine the properties of individual fragments, since such fragments need to have very high internal stability to preserve their functions at high temperatures.

This invention demonstrates that the unique enzymatic properties of DNA topoisomerase V, such as its ability to work in high salt concentrations and its high processivity, can be transferred to other DNA processing enzymes. It has been shown herein that, if properly positioned, HhH repeats derived from Topo V not only greatly increase the salt resistance of the Stoffel fragment of Taq polymerase, but also confer processivity on hybrid polymerases in high salt concentrations where Taq polymerase works distributively and/or is inhibited. This invention further demonstrates that, if properly positioned, HhH repeats derived from Topo V not only restore the processivity of Pfu polymerase or fragments thereof to the level of Pfu polymerase, but also confer processivity on hybrid Pfu polymerases in high salts where Pfu polymerase works distributively and/or is inhibited.

It is important to note that the alterations of the core DNA polymerase processivity did not correlate with the overall charge of the attached polypeptide chain. For example, at neutral pH, both Taq DNA polymerase and KlenTaq have an overall negative charge of NH₂-terminal polypeptides adjacent to the Stoffel fragment, but TopoTaq has a positively charged NH₂-terminus. TaqTopoC1 has a negatively charged polypeptide attached to the C-terminus of the Stoffel fragment, but TaqTopoC2 and TaqTopoC3 have positively charged attachments. Therefore, electrostatic interactions with specific amino acid residues rather than the overall charge of the linked domains are responsible for the stability of DNA-polymerase complexes in salts.

Inhibition of Taq polymerase, Pfu polymerase, and the chimeric polymerases by the salts as demonstrated herein reveals complicated cooperative binding of DNA by the polymerase catalytic domains with HhH repeats. Such interaction between the Taq polymerase domain and Topo V domains provides full DNA polymerase activity of the chimeras at high salt concentrations. With the exception of TaqTopoC3, introduction of C-terminal domains of Topo V into the hybrid proteins significantly extends the range of salt concentrations for the polymerase activity. The effect is caused by the increase of both Kᵢ and α, allowing chimeras to maintain their full activity at high salt concentrations. Such behavior indicates that in the catalytic domain the incorporation of dNMPs is not affected, even at high concentrations of salts. Rather, binding of DNA substrate is impaired because of competition between DNA and the anions for the positively charged amino acid residues of the proteins. Raising the number of HhH motifs from 11 to 23 at the COOH terminus of the Stoffel fragment makes the hybrid enzymes progressively more resistant to salts.

The methods of this invention may also be extended to other DNA polymerases in addition to TaqDNA polymerase and PfuDNA polymerase. For example, this invention includes both DNA-dependent polymerases and RNA-dependent polymerases.

The methods of this invention may be extended to other enzymes. For example, this invention contemplates linking other DNA processing enzymes, for example reverse transcriptases, restriction endonucleases, hecases, and topoisomerases, or fragments thereof, to HhH domains. The incorporation of topologically linked and physically bound HhH repeats should not necessarily perturb the structurally defined catalytic domains; rather, they may act in concert. The ability to engineer hybrid enzymes can also be useful for generating enzymes with new properties for practical applications. Taq DNA polymerase [26] carries out fast and processive synthesis of DNA. It has high thermostability, but its activity is inhibited at elevated salt concentrations. Pfu polymerase has a high fidelity, but it is salt-sensitive and is not processive. Moreover, it appears the majority of commercially available thermostable DNA polymerases show little or no activity at NaCl or KCl concentrations over 80 mM [25, 27]. Their resistance to salts could be increased by fusion with Topo V HhH domains.

### EXAMPLE 1

### Construction of Chimeric Taq DNA Polymerases

All plasmids were constructed by common subcloning techniques and propagated in DH5α (Invitrogen) strain of E. coli. Taq DNA polymerase and its KlenTaq variant were purchased from Roche Applied Science and from GeneCraft (Munster, Germany), respectively, and the Stoffel fragment was obtained from Applied BioSystems.

TaqTopopET21d—The polymerase chain reaction was used to amplify segments of the M. kandleri top5 gene covering amino acids 685-984 from pAS6.5 plasmid [1] and the Taq polymerase gene covering amino acids 290-832 (Stoffel fragment) from pTTQ plasmid (gift of Dr. G. Belov). In the case of the top5 gene fragment, the linker 5′-GCCTACGACGTAGGCGCC-3′ (SEQ ID NO. 1) (translated into AYDVGA) was added at the 3′ end of the fragment. The 5′ end of the top5 gene fragment contained the Nde I restriction site with the initiating AUG codon, while two stop codons were placed at the 3′ end of the Taq fragment followed the HindIII restriction site. The 3′ end of the top5 fragment was blunt ligated to the 5′ end of the Taq fragment, digested with Nde I-HindIII and the resulting DNA was cloned into the pET21d expression vector (Novagen).

TaqTopoC1-pET21d, TaqTopoC2-DET21d and TaqTopoC3-pET21d—The Taq polymerase gene fragment covering amino acids 279-832 was amplified by PCR from pTTQ plasmid using primers with incorporated EcoRI and HindIII sites. The 1684 bp fragment was then digested with EcoRI and HindIII and cloned into EcoRI-HindIII digested pBlueScript KSII vector (Stratagene) to yield the Stoffel-BS vector. Next, segments of the top5 gene covering amino acids 384-984 (C1), 518-984 (C2) and 676-984 (C3) and including the top5 terminating codon were PCR amplified from pAS6.5 plasmid using primers with incorporated HindIII and SalI sites. These PCR products were digested with HindIII and SalI and subcloned into the pBS KSII vector (Stratagene) to yield Stoffel-C1, Stoffel-C2 and Stoffel-C3 vectors. The inserts were cut out by HindIII and SalI digestion and cloned into the HindIII-SalI digested Stoffel-BS plasmid making Stoffel-C1, Stoffel-C2 and Stoffel-C3 fusions with AAGCTT (SEQ ID NO. 2) (HindIII site) linker sequence. The resulting combined Stoffel-C1, Stoffel-C2 and Stoffel-C3 inserts were cut out by NcoI (the NcoI site was introduced by PCR primer used for generating Stoffel-BS plasmid) and SalI and cloned into pET21d vector to result in expression vectors TaqTopoC1-pET21d (“TaqTopoC1”), TaqTopoC2-pET21d (“TaqTopoC2”) and TaqTopoC3-pET21d (“TaqTopoC3”). All subcloned sequences that had been subjected to polymerase chain reaction were sequenced to confirm proper position of initiation signals and to ensure that no other mutations were introduced.

E. coli strain BL21 pLysS (Novagen) was transformed with expression plasmids. For each DNA polymerase, 2 L of LB medium containing 100 μg/ml of ampicillin and 34 μg/mL of chloramphenicol was inoculated with transformed cells, and the protein expression was induced by adding 1 mM isopropylthio-β-galactoside (IPTG) and carried out at 37° C. for 3 hours. The cells were harvested and dissolved in 100 ml of lysis buffer containing 50 mM Tris-HCl pH 8.0, 1 mM EDTA, 5 mM β-mercaptoethanol, and protease inhibitors (Roche). The lysate was centrifuged at 12000 g for 30 minutes, heated at 75° C. for 30 minutes, and centrifuged again at 15000 g for 1 hour. The supernatant was filtered through a 0.22 μm Millipore filter and applied on a heparin high trap column (APB) equilibrated with 0.5 M NaCl in 50 mM Tris-HCl buffer, pH 8.0, containing 2 mM β-mercaptoethanol. The column was washed with 100 ml of the same buffer, and the protein was eluted in 20 mL of 0.75 M NaCl in 50 mM Tris-HCl buffer, pH 8.0, with 2 mM β-mercaptoethanol.

### EXAMPLE 2

### Construction of PfuC2 Chimera

PfuC2-pET21d-2325 bp Pfu DNA polymerase cds was subdivided into two parts, 978 and 1353 bp-long, and each one was individually PCR-amplified from Pyrococcus furiosus genomic DNA. The NcoI-EcoRI-digested upper PCR fragment (NcoI site was introduced in the PCR primer) was cloned into NcoI-EcoRI sites of modified pBlueScript II SK− vector (the modified vector carries NcoI-BglII recognition sites inserted between PstI and EcoRI sites of the polylinker sequence). The EcoRI-HindIII-incompletely digested lower PCR fragment (HindIII site was introduced in the primer, an additional HindIII site is present in the Pfu cds) was cloned into EcoRI-HindIII sites of the modified pBlueScript II SK vector. Sequencing of several upper and lower inserts revealed clones carrying the correct sequences. The upper insert was cloned in the NcoI and EcoRI sites of the plasmid, which already carried the lower insert, thus joining both parts of the Pfu cds together. The Pfu cds was cut out by NcoI-HindIII digestion (HindIII-digestion was incomplete), the C-terminal C2 domain of Topo V was cut out from the top5-C2-p BlueScript II SK plasmid by HindIII-SalI double digestion, and both parts were ligated with NcoI-SalI-digested pET21d vector. The resulting expression construct was verified by restriction digestion. The final protein starts with Met-Val instead of Met-Ile (as it is in the wild-type Pfu polymerase) at its N-terminus and contain the Lys-Leu linker between Pfu polymerase and the C2 domain of Topo V.

### EXAMPLE 3

### Primer Extension Assay

An enzymatic polymerization in primer extension reactions can be described by Equation 5:

$$E + {S}_{0} ↔ E * {S}_{0} \to E * {S}_{1} \to E * {S}_{2 } ⋯ \to E * Si ⋯ \to E + Pn \\ ⇕ ⇕ ⇕ \\ E + {S}_{1} E + {S}_{2} E + Si \qquad Eq . 6$$

in which each product of a consecutive step of the polymerization, except the last one, is, in turn, a substrate (Sᵢ) for the next step. Rates of accumulation of these intermediate substrates Sᵢ and their complexes with the enzyme (or products of each step of polymerization, Pᵢ; [Sᵢ]≡[Pᵢ]) can be expressed by Equations 6-9:

$$\frac{ⅆ [ {P}_{1} ]}{ⅆ t} = {v}_{1} - {v}_{2} ; \\ \frac{ⅆ [ {P}_{2} ]}{ⅆ t} = {v}_{2} - {v}_{3} … ; \\ \frac{ⅆ [ {P}_{n - 1} ]}{ⅆ t} = {v}_{n - 1} - {v}_{n} ; \\ \frac{ⅆ [ {P}_{n} ]}{ⅆ t} = {v}_{n} \qquad Eqs . 7 \text{-} 10$$

where vᵢ are rates of appearance of a product for each step of polymerization. The total rate of accumulation of the products calculated for the sum of concentrations of all extended species in Equation 1, where P=Σ[Pᵢ] is obtained by Equation 11:

$$\frac{ⅆ P}{ⅆ t} = \frac{ⅆ ( \sum [ {P}_{t} ] )}{ⅆ t} = \sum ( \frac{ⅆ {P}_{t}}{ⅆ t} ) = {v}_{1} . \qquad Eq . 11$$

That is, dP/dt is equal to the rate of attachment of the first nucleotide (v₁). Therefore, the initial rates for nucleotide incorporation into primer:template junctions can be obtained from time courses for accumulation of extended products by extrapolating dP/dt to t=0.

A primer extension assay was developed using a fluorescent duplex substrate containing a primer-template junction (PTJ). The duplex was prepared by annealing a 5′-end labeled with fluorescein 20-nt long primer with a 40-nt long template as shown:

```
                            *F1-gtaatacgactcactataggg →
                                    |||||||||||||||||||||
tttttgctgccggtcacttaacattatgctgagtgatatccc
```

extension
aaaaaccccc

where SEQ ID NO. 3=gtaatacgactcactataggg; and SEQ ID NO. 4=tttttctgcgcgtcacttaacattatgctgagtgatatcccaaaaaccccc.

DNA polymerase reaction mixtures (15-20 μL) contained dATP, dTTP, dCTP, and dGTP (1 mM each), 4.5 mM MgCl₂, detergents Tween 20 and Nonidet P-40 (0.2% each), fixed concentrations of PTJ-duplex, other additions, as indicated, and appropriate amounts of DNA polymerases in 30 mM Tris-HCl buffer pH 8.0 (25° C.). The background reaction mixtures contained all components except DNA polymerases. Primer extensions were carried out for a preset time at 75° C. in PTC-150 Minicycler (MJ Research). Samples (5 μL) were removed and chilled to 4° C. followed by immediate addition of 20 μL of 20 mM EDTA. The samples were desalted by centrifugation through Sephadex G-50 spun columns, diluted, and analyzed on an ABI Prism 377 DNA sequencer (Applied BioSystems).

For each sample, raw data were extracted from the sequencer trace files with a program Chromas v. 1.5 (Technelysium Pty Ltd., Australia), and the fluorescent signals were analyzed by nonlinear regression data analysis programs written in FORTRAN. The programs applied Powell [12] algorithms to approximate the signals by a number of Gaussian peaks and calculate integral fluorescent intensities for each product peak. The total amount of fluorescent products for each time of incubation was determined, and the initial rates of extension were calculated as described [13].

### EXAMPLE 4

### Processivity Assays for DNA Polymerases

The microscopic processivity parameter Pₙ can be determined from signals of 5′-end labeled extended products that appeared in a template-directed DNA synthesis carried out under single-hit conditions of the assay as shown in Equation 12 [14]:

$$Pn = {\sum }_{i = 1}^{{n}_{max} - n} {I}_{n + i} / {\sum }_{i = 0}^{{n}_{max} - n} {I}_{n + i} \qquad Eq . 12$$

where Iₙ₊₁ are gel band intensities for oligonucleotides extended by subsequent additions of deoxynucleotides.

The single-hit conditions occur in the initial period of a primer extension reaction if the PTJ is in great excess over DNA. Since during the initial period of a primer extension reaction (Δt)Iₙ₊ᵢ=dlₙ₊ᵢ/dt*Δt, pₙ can be also expressed through initial rates of appearance of extended products as shown in Equation 13:

$$\\ {Pn}^{′} = {\sum }_{i = 1}^{{n}_{max} - n} {vl}_{( n + i )} / {\sum }_{i = 0}^{{n}_{max} - n} {vl}_{( n + i )} \\ where v ( {l}_{n + i} ) = ⅆ {l}_{n + i} / ⅆ t . \qquad Eq . 13$$

For processivity assays, the primer extension reactions were carried out and analyzed as described above, but after determination of the amount of extended products, the initial rates for appearance of each extended primer were calculated [13]. The processivity for each position of the template was then determined using Equation 13, and the processivity equivalence parameter, Pₑ, was calculated for each reaction.

### EXAMPLE 5

### Dependence of Primer Extension Rate on Substrate Concentration

The kinetics of the reaction of primer extension at steady-state is described by a Michaelis equation in Scheme 1:

$${v}_{1} = {k}_{app} * [ {E}_{t} ] * [ {S}_{0} ] / ( {K}_{mapp} + [ {S}_{0} ] ) , \\ where {k}_{app} = {k}_{1} / {\sum }_{i = 1}^{n} {\prod }_{j = 1}^{i} {k}_{j} / ( {k}_{b ( - j )} + {k}_{j + 1} ) and \\ {Km}_{app} = {Km}_{0} / {\sum }_{i = 1}^{n} {\prod }_{j = 1}^{i} {k}_{j} / ( {k}_{b ( - j )} + {k}_{j + 1} ) . \qquad Scheme 1$$

At very low concentrations of Sₒ (Kmₐₚₚ>>[Et]*[Sₒ]), complexes with several molecules PTJ bound per molecule of polymerase disappear, V1≈kₐₚₚ/Kmₐₚₚ*[Eₜ]*[Sₒ], and the initial rates of reactions are proportional to [PTJ] (as in FIG. 6). Since kₐₚₚ/Kmₐₚₚ=k_{bo}*k1/(k_{b-o}+k₁), the slope of a line in FIG. 6 is equal to k_{bo}*k1/(k_{b-o}+k₁)*[Eₜ].

The stoichiometry of substrate binding in the presence of betaine is estimated to be greater than 1 mol/(mol polymerase active sites), however no inhibition by high substrate concentrations is observed, indicating that active mixed complexes are formed.

In addition, as k₁/(k_{b-o}+k₁) represents a microscopic processivity parameter in the reaction of addition of the first nucleotide, v₁=k_{bo}*pₒ*[Eₜ][Sₒ]. Hence, this finding demonstrates that the dependencies of initial rates in FIG. 7 are functions of the change in both the rates of binding of the DNA substrate to the polymerases and the processivity of synthesis. At low salt concentrations, the values of processivity are quite close to unity. Under these conditions, it was possible to estimate rate constants for bimolecular association of the enzymes with substrates (k_{bo}) that were in the range of 0.4-1.6×10⁶ M⁻¹s⁻¹.

### EXAMPLE 6

### Studies of Thermostability of DNA Polymerases

Proteins in 25 μl of the 20 mM Tris-HCl buffer (pH 8.0 at 25° C.) containing the indicated concentrations of salts and betaine were incubated in PTC-150 Minicycler (MJ Research) at 95° C. or 100° C. Samples (4 μL) were removed at defined times of incubation and assayed for primer extension activity for 10 minutes with 0.72 μM PTJ.

### EXAMPLE 7

### Dye Primer Cycle Sequencing with TopoTaq Chimera

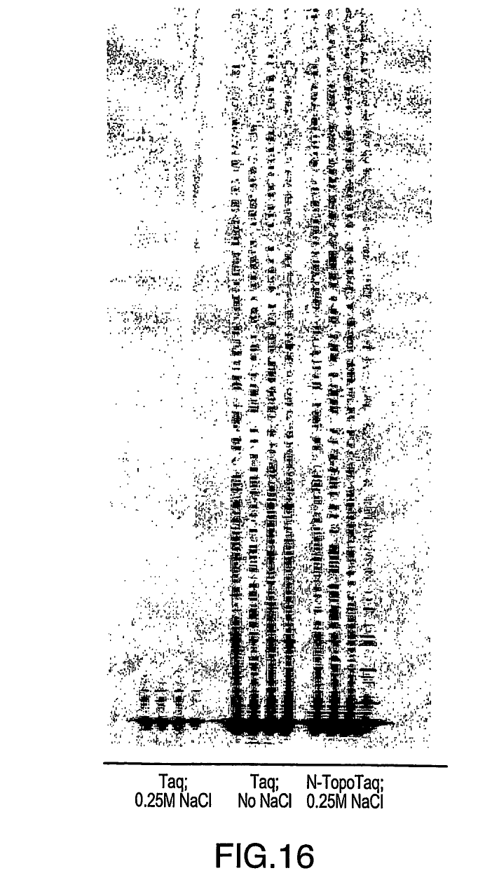

Cycle sequencing with ALF M13 fluorescent primer (APB) was performed essentially as recommended by APB for natural Taq DNA polymerase and 5′-end labeled primers (APB's cycle sequencing kit). Reaction mixtures contained dATP, dTTP, dCTP, and d7-deaza dGTP (11.43 μM each), single stranded M13mp18 (+) Strand DNA (10 ng/μL), ALF M13 Universal Primer (0.6 μM), proteins, and indicated salt concentrations. Four aliquots of each reaction mixture were mixed with four terminator dideoxynucleotides (ddATP, 222.9 μM; ddCTP, 85.7 μM; ddGTP, 6.9 μM; ddTTP, 242.9 μM). The cycling protocol included an initial denaturation for 2 minutes at 94° C., then melting 40 seconds at 94° C., annealing for 40 seconds at 55° C., and elongation for 1.5 minutes at 72° C. for 30 cycles. After chilling, desalting, evaporation and dissolving in the ABI loading buffer, the samples were analyzed on an ABI Prism 377 DNA sequencer. FIG. 16 shows the dye primer sequencing using N-TopoTaq in the presence of NaCl. The panel displays a urea gel picture of single-stranded DNA M13mp18(+) sequencing with ALF M13 Universal fluorescent primer using Taq DNA polymerase and N-TopoTaq. Salt concentrations are indicated on the gel.

### EXAMPLE 8

### Dependence of Rate of Primer Extension Reaction on the Reaction Temperature for Chimeric DNA Polymerases

A primer extension assay was developed with a fluorescent duplex substrate containing a primer-template junction (PTJ). The duplex was prepared by annealing a 5′-end labeled with fluorescein 40-nt long primer with the following 50-nt long template:

F1*
|
gtgtggtggtggtggtggtggtggtggtggtggtggtggt
||||||||||||||||||||||||||||||||||||||||
cacaccaccaccaccaccaccaccaccaccaccaccaccaaaaaattttt

where SEQ ID NO. 5=gtgtggtggtggtggtggtggtggtggtggtggtggtggt; and SEQ ID NO. 6=cacaccaccaccaccaccaccaccaccaccaccaccaccaaaaaattttt.

DNA polymerase reaction mixtures (5 μL) contained dATP, dTTP, dCTP, and dGTP (1 mM each), 4.5 mM MgCl₂, detergents Tween 20 and Nonidet P-40 (0.2% each), 0.32 μM PTJ, potassium glutamate, as indicated, and appropriate amounts of DNA polymerases in 30 mM Tris-HCl buffer pH 8.0 (25° C.). The background reaction mixtures contained all components except the polymerases. Primer extensions were carried out in PTC-150 Minicycler (MJ Research) for specified times in the range of temperatures 50-105° C. (with 5 degree intervals). For each reaction, 1.5 μL samples were removed after 3, 6, and 9 minutes of incubation and chilled to 4° C., followed by immediate addition of 20 μL of 20 mM EDTA. The samples were desalted by centrifugation through Sephadex G-50 spun columns, diluted, and analyzed on an ABI Prism 377 DNA sequencer (Applied BioSystems). For each sample, raw data were extracted from the sequencer trace files and the integral fluorescent intensities for each product peak were calculated. The total amount of fluorescent products for each time of incubation was determined, and the initial rates of extension were calculated from the progressive accumulation of the products as described earlier [13].

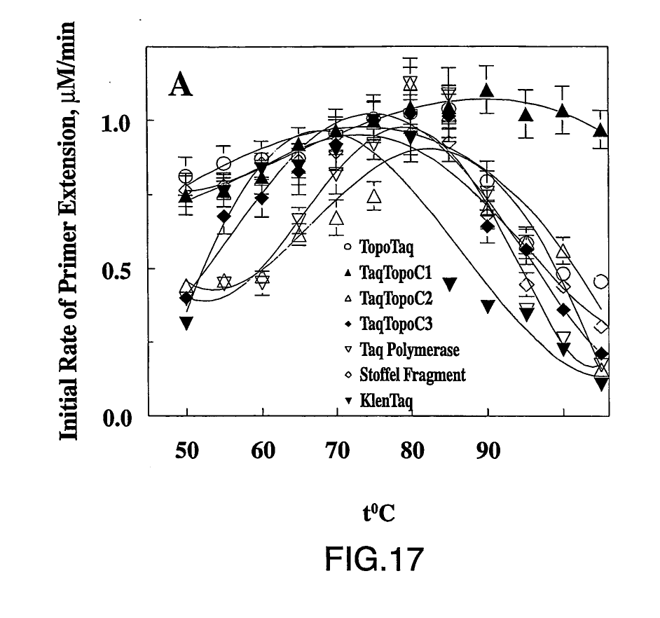

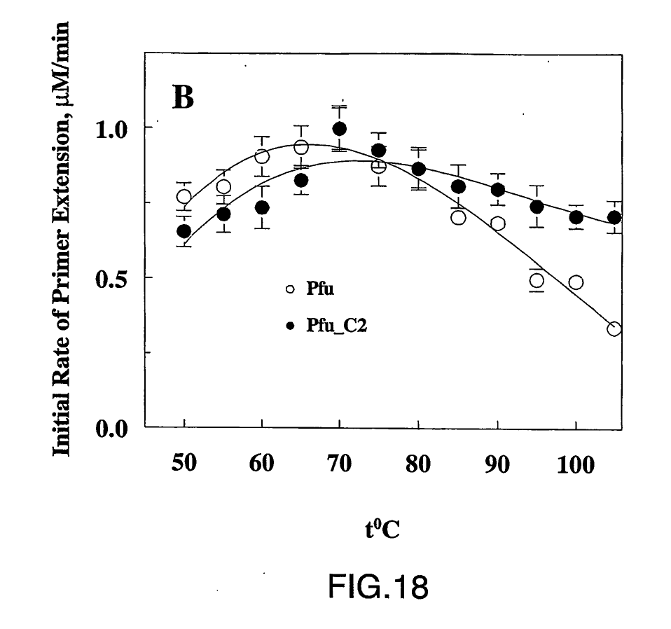

The activities of Taq DNA polymerase, the Stoffel fragment, Pfu polB, and the hybrid polymerases were measured at different temperatures. The dependencies of the initial rates of primer extension reactions on the reaction temperature were plotted for enzymes with Taq polymerase catalytic domain in FIG. 17, and for enzymes with Pfu polymerase catalytic domain in FIG. 18. The rates for TopoTaq, TaqTopoC1, TaqTopoC2, TaqTopoC3, and PfuC2 were measured in 0.25 M potassium glutamate; reactions with Pfu polymerase, Taq polymerase, and its Stoffel, and Klentaq fragments were carried out without salts added.

The results show that the catalytic domains of both Taq and Pfu DNA polymerases can carry out polymerization of DNA at temperatures as high as 105° C., with virtually the same efficiency as at lower temperatures. As shown in FIG. 1, the activity at high temperatures is provided by DNA binding domains of Topo V properly positioned in the chimeras, rather than by addition of potassium glutamate. It is likely that the efficient binding of DNA at high temperatures occurs between double stranded part of PTJ and HhH motifs of superdomain C2 of Topo V. The results also imply that the rate of primer extension is much higher then the rate of melting the PTJ substrate even at the highest temperatures in the assay. It is also possible that DNA polymerases can stabilize PTJ duplexes during DNA synthesis.

### EXAMPLE 9

### Modeling Domains of TopoV and Design of Chimeras

Although the crystal structure of Topo V is not known, current biochemical information suggests that the HhH motifs of the protein are folded into distinct units, which are further organized into bigger structures as it was revealed by limited proteolysis (9, 10). Computer modeling was used for 3D structures of the individual TopoV HhH domains based on structural information obtained for other proteins with HhH domains. Use of protein 3D modeling servers, such as SwissModel (29-32) or Geno3D (33) with the automatic mode of sequence recognition allowed only for modeling of TopoV domain G because of its high similarity to RuvA DNA binding domain. In all other cases, low sequence similarity of TopoV domains to the proteins with known structures prevented finding out a proper template for modeling. Therefore, the structural data bank was screened for non-redundant proteins with double HhH repeats (the majority of structures already existed in EBI and NCBI databases); also, structures found by Shao and Grishin (5) were added. The found proteins were checked against Fold classification based on Structure-Structure alignment of Proteins (FSSP, (34)) database for closely related proteins. If structures of the corresponding protein-DNA complexes with resolution <3 Å existed, then these were used instead of the structures of individual proteins.

Seven proteins were found and used as templates: 1 bpy (human DNA polymerase β), 1c7y (holiday junction DNA helicase RUVA; Escherichia coli), 1coo (RNA polymerase alpha subunit; Escherichia coli), 1dgs (NAD⁺-dependent DNA ligase; Thermus filiformis), 1ebm (human 8-oxoguanine DNA glycosylase), 2abk (endonuclease III; Escherichia coli), and 2pjr (helicase PCRA; Bacillus stearothermophilus). The structures of HhH domains excised from the Protein Data Bank files served as templates for TopoV domain modeling with SwissModel server. All TopoV HhH domains, except domain J, were successfully modeled with at least one of the templates tried. If the server suggested several structures, the one with the lowest calculated free energy was chosen. Domain J, which had a too short similarity range to 1dgs according to parameter of the server, was folded by Swiss-PDBViewer using the 1dgs structure as a template, followed by an energy minimization procedure in vacuum with GROMOS (35). Domain L was found to have two overlapping parts; one (amino acids 910-940 in TopoV) was similar to the DNA ligase HhH domain (1dgs), while the other (amino acids 959-984) was similar to the N-terminal HhH domain of human DNA polymerase β. The intermediate loop (amino acids 941-958) could be folded using both templates, and it had shown almost identical conformation in both cases. Therefore, the two folded parts of the domain were joined in an orientation that provided the best overlay of the residues in the intermediate loop, and the resulting structure was subjected to the energy minimization procedure.

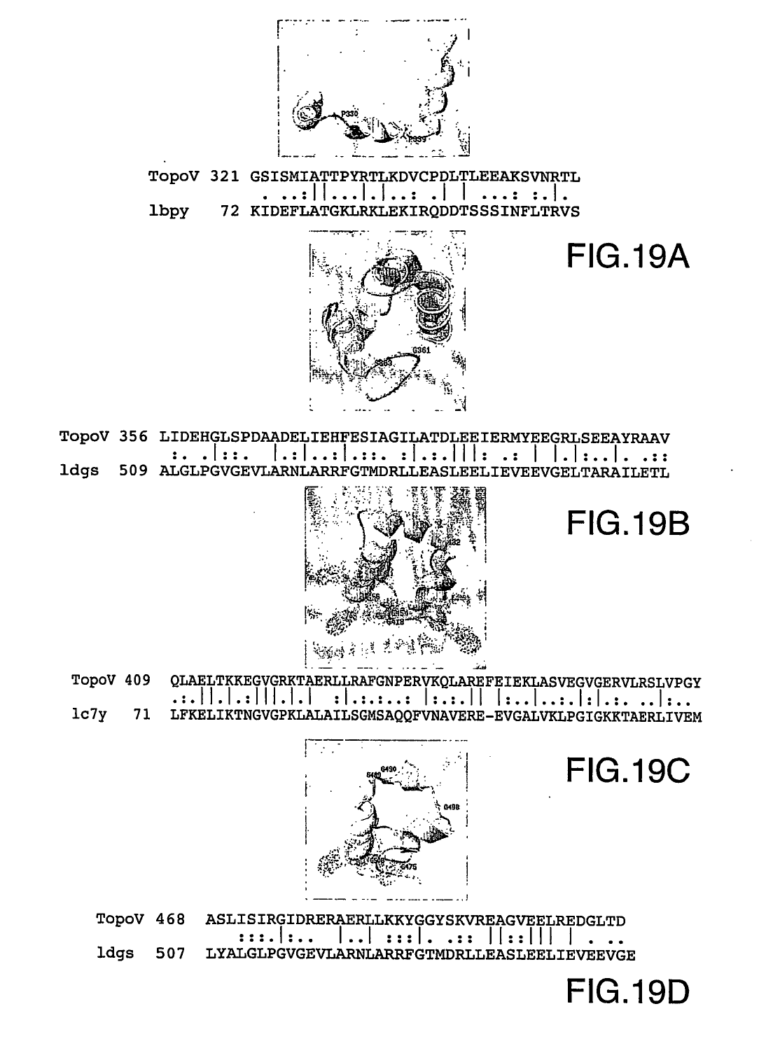

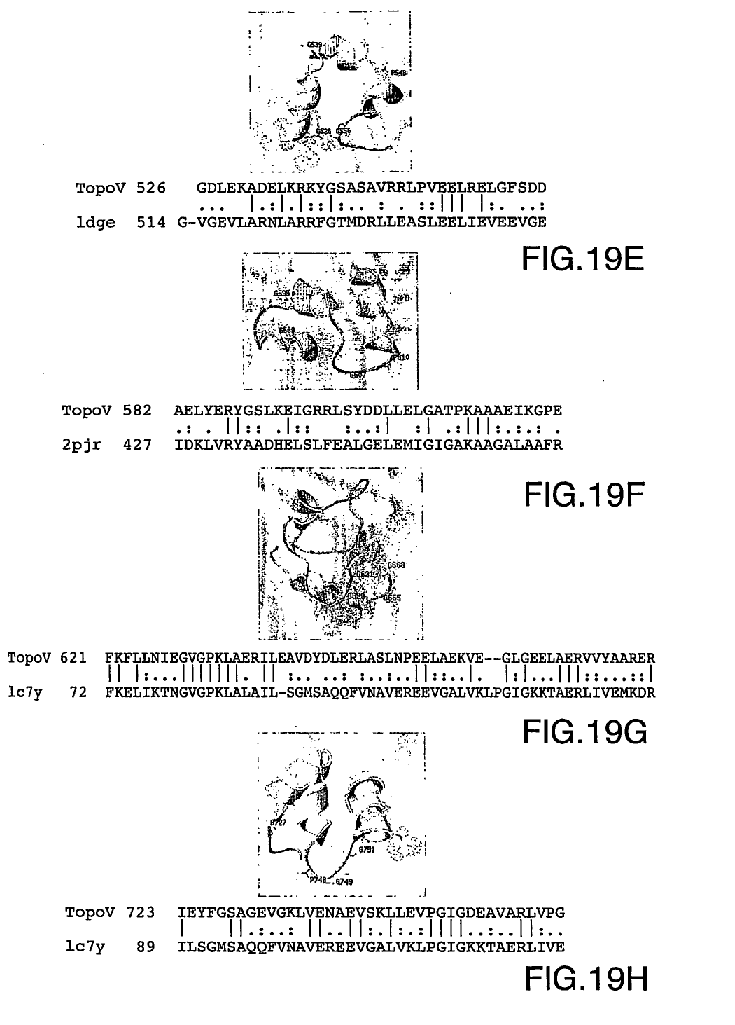

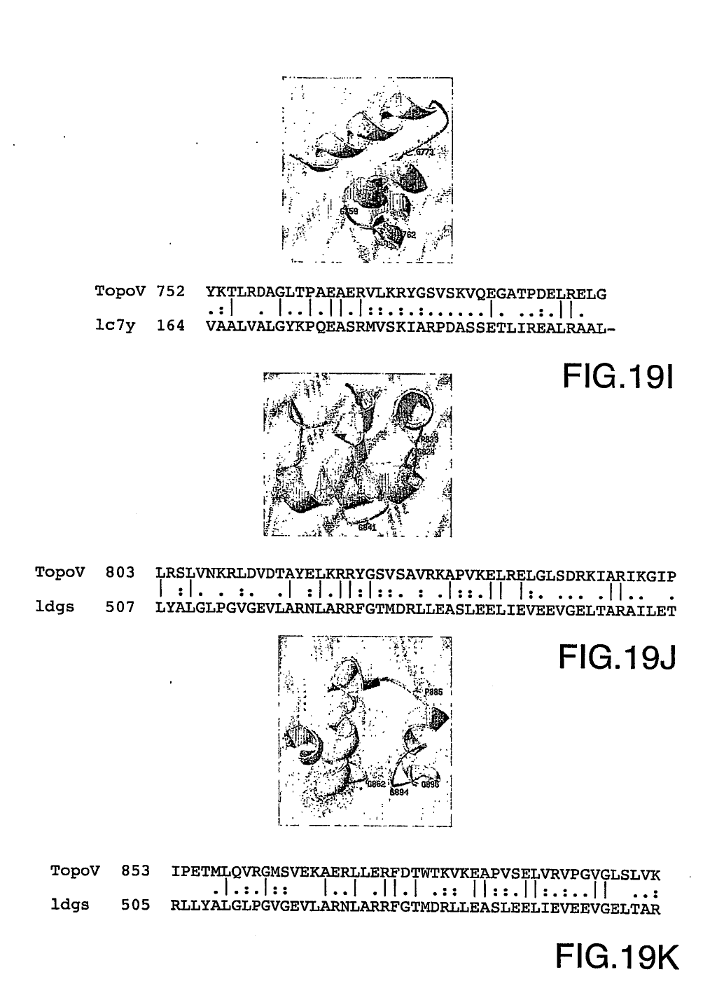

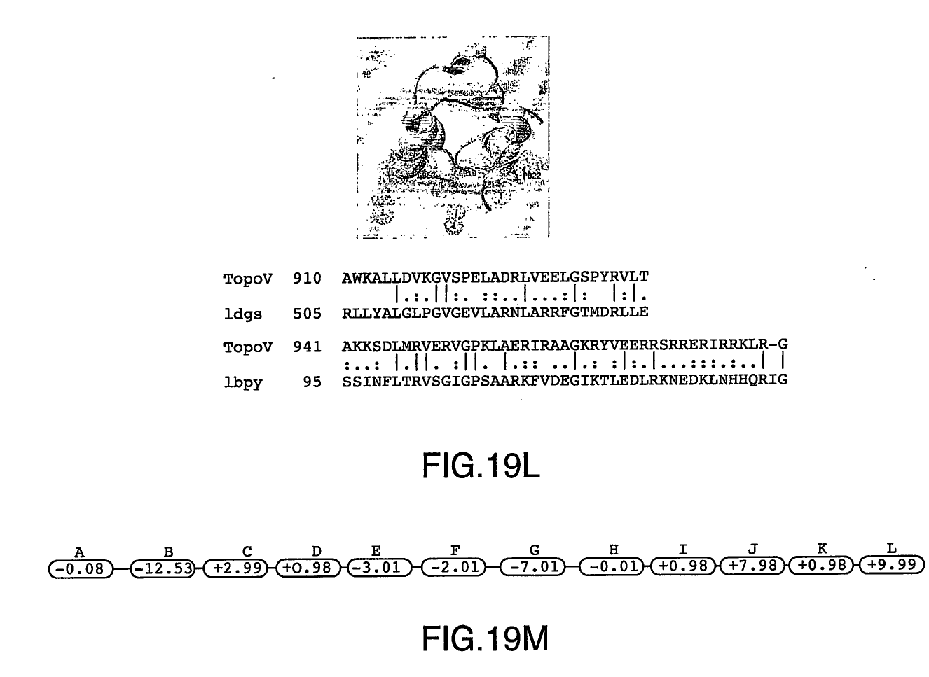

FIGS. 19A-L summarize the results of TopoV domain modeling, along with structural alignment of the TopoV HhH motifs with the template HhH domains. Structural diagrams for the models of HhH domains A-L are shown along with the structural alignment of the TopoV domains and HhH domains in the proteins with the solved X-ray structures. In the alignments, the amino acids that were shown to have contacts with DNA in proteins with the known structures are marked red. The analogous amino acids that likely bind DNA in proteins with the known structures are marked blue. Indicated in the models are the van der Waals radii of the side chains that might contact DNA and the conserved glycine and proline residues, which are important for formation of HhH structures. The panel also shows the calculated distribution of partial charge along the TopoV HhH domains at pH 7.0 (FIG. 19M). TopoV repeats B, D, E, J, K, and L could be folded using the ligase (1dgs) HhH domain as a template. Sequences in the domains C, G, H, and I had similarity to the helicase RuvA (1c7y) HhH fold. Domains A and L had similarity to polymerase β (bpy); domain F was found to be similar to one in helicase PCRA (2pjr). No similarity was detected with HhH domains from RNA polymerase alpha subunit (1coo), glycosylase (1ebm), or endonuclease (2abk).

As the structure of the helicase RuvA complex with DNA is known (1c7y), we located the conserved amino acid of the TopoV domains that correspond to the amino acids of RuvA in contact with DNA, according to the structural alignment. Those were found in domains C, G, and H, but not in I. The sequence in domain F was similar to a region in helicase PCRA (2pjr) that did not include any amino acid residue contacting with DNA. Likewise, the domain A had similarity to a part of polymerase β that did not contact DNA in 1bpy. Six out of twelve TopoV domains have similarity to the ligase HhH domain, however the structure of the ligase-DNA complex is unknown. Moreover, the commonly used structural alignments produced by programs DALI (36) or VAST (37) did not show any similarity of the specific part of 1dgs, which was chosen by both SwissModel and Geno3D servers as a template for TopoV, to any other protein with known contacts with DNA. However, we successfully used a combinatorial extension (CE) approach (38) provided by a server at San Diego Supercomputer Center (http://cl.sdsc.edu/ce.html) and obtained the structural alignment of the ligase with helicase RuvA and polymerase β (structures 1c7y and 1bpy).

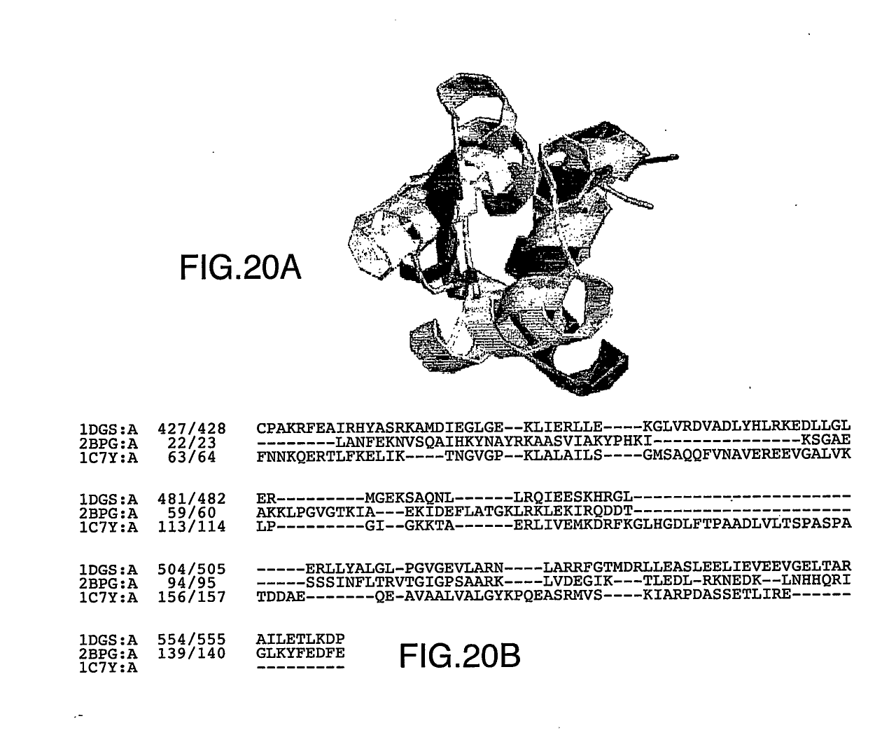

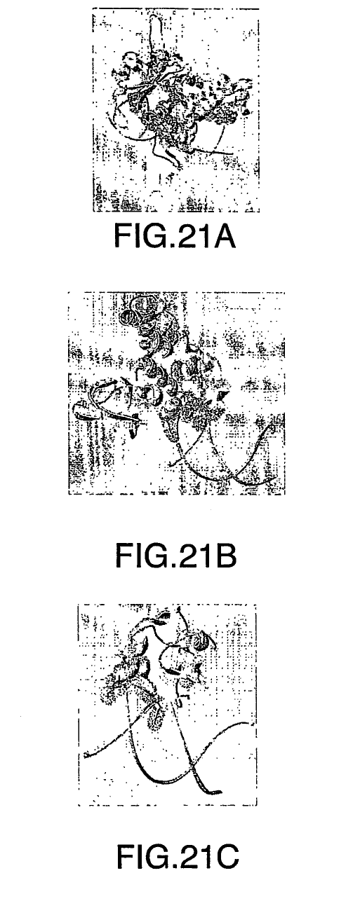

FIGS. 20A and 20B display the structural alignments found by CE. FIG. 20A shows the structural alignment of NAD⁺-dependent DNA ligase from Thermus filiformis, human DNA polymerase β, and FIG. 20B shows the structural alignment of holiday junction DNA helicase RuvA from E coli and corresponding structure-based sequence alignments. Sequence alignment is based on assembled pair-wise structure alignments of 1DGS_A with its neighbors. The light color indicates non-aligned residues in structural neighbors. Position numbers according to sequence (starting from 1) and according to PDB are given as SSSS/PPPP, SSSS—sequence, PPPP—PDB. The proteins in FIG. 20A are colored as in the sequence alignment in FIG. 20B. It is important that the ligase HhH domain, which is the one with the highest similarity to TopoV domains, also contains a well conserved amino acid sequence that is responsible for DNA binding in polymerase β seems very likely that this sequence (colored blue in FIG. 19) binds DNA in the ligase and in similarly folded domains of TopoV. Consequently, we located regions of TopoV domains B, D, E, J, K, and L, which have ligase-like folds, and marked the similar conservative residues along with adjacent basic amino acid residues, as the expected sites for DNA binding.

We designed four chimeric proteins consisting of the catalytic (Stoffel) fragment of Taq DNA polymerase and three C-terminal amino acid sequences of TopoV, which include repeats B-L, E-L, and H-L, respectively. These sequences sequentially encompass the complete structures produced by the HhH domains in TopoV, as revealed by limited proteolysis (10), starting from the COOH-terminal H-L formation. As in TopoV, we attached the three sequences to the COOH-termini of the polymerase domain.

FIGS. 21A-C show structures of HhH motifs bound to DNA molecules in protein-DNA complexes. FIG. 21A is complexes of DNA with human DNA polymerase β (1bpy), FIG. 21B is E. coli helicase RUVA (1c7y), and FIG. 21C is the proposed structure for DNA bound to the modeled Topo V domain L.

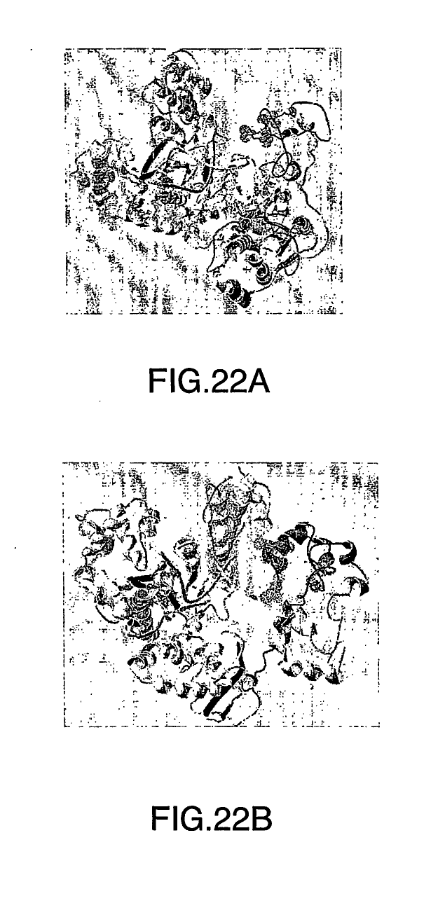

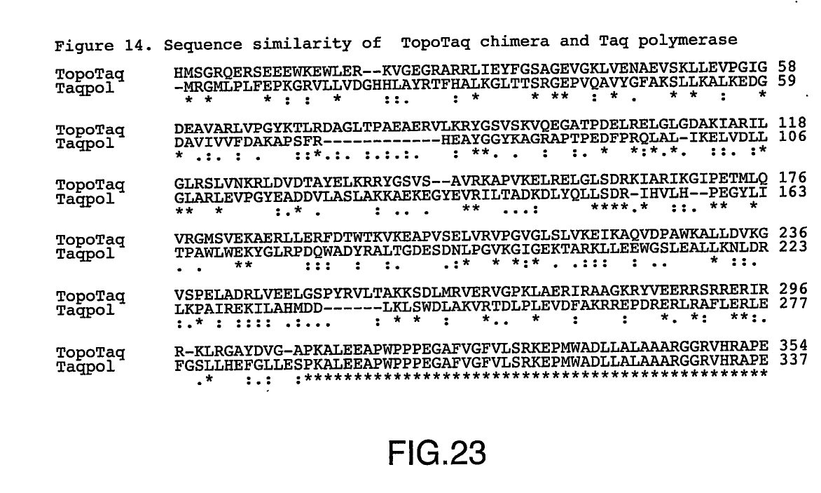

It is known that Taq polymerase contains an HhH fold in the 5′→3′ exonuclease domain; however no direct contacts of this structure with DNA have been demonstrated. The X-ray structure of Taq polymerase with DNA shows the conformation of the protein with the HhH domain at distant position with respect to the DNA substrate (1tau, “open” conformation). In contrast, the X-ray structure of the protein with the HhH domain in proximity to the polymerase active site is solved without the DNA (1cmw, “closed” conformation). We overlaid the catalytic domains of these structures and position the DNA substrate from 1tau into 1cmw. It was possible further to bring the Taq HhH domain in contact with the DNA, after a relatively small turn of the entire exonuclease domain. The resulting structure is shown in FIG. 22A. Similarly, a sequence containing HhH domains H-L could be attached through a suitable linker to the NH₂-termini of the catalytic domain of Taq polymerase to bring the TopoV HhH domain L in proximity to the DNA substrate (FIG. 22B). Therefore, a TopoTaq chimera was designed, such that the entire TopoV structure containing HhH motifs H-L has been fused with NH₂-termini of the Stoffel fragment through a linker.

The models were built with Swiss-PDBViewer, followed by an energy minimization procedure in vacuum for relaxation of the structures. Building of the model in in FIG. 22A is described in the text. For the model in FIG. 22B, the structures for domains L and K (FIG. 19) were bound, and the energy of the resulting structure was minimized. After attaching the linker sequence used in TopoTaq, the energy of the resulting structure was minimized again, and the modeled domains were connected to the structure of the Stoffel fragment with DNA (1qsy), followed by energy minimization. In the modeled proteins, the catalytic domain of Taq polymerase is shown in green, Taq polymerase HhH domain and Topo V HhH domain L are colored violet, the rest of the Taq polymerase 5′ to 3′ exonuclease domain and TopoV domain K are colored dark yellow, and the linker in the TopoTaq chimera is colored dark orange.

In addition, a significant homology of the entire 5′ to 3′ exonuclease domain of Taq polymerase to the sequence containing the TopoV HhH repeats has been found, which might provide better interactions of the TopoV polypeptide with the catalytic domain of Taq polymerase (FIG. 23).

### EXAMPLE 10

### DNA Topoisomerase V Sequences (DNA and Protein) from Methanopyrus Strain Tag 11

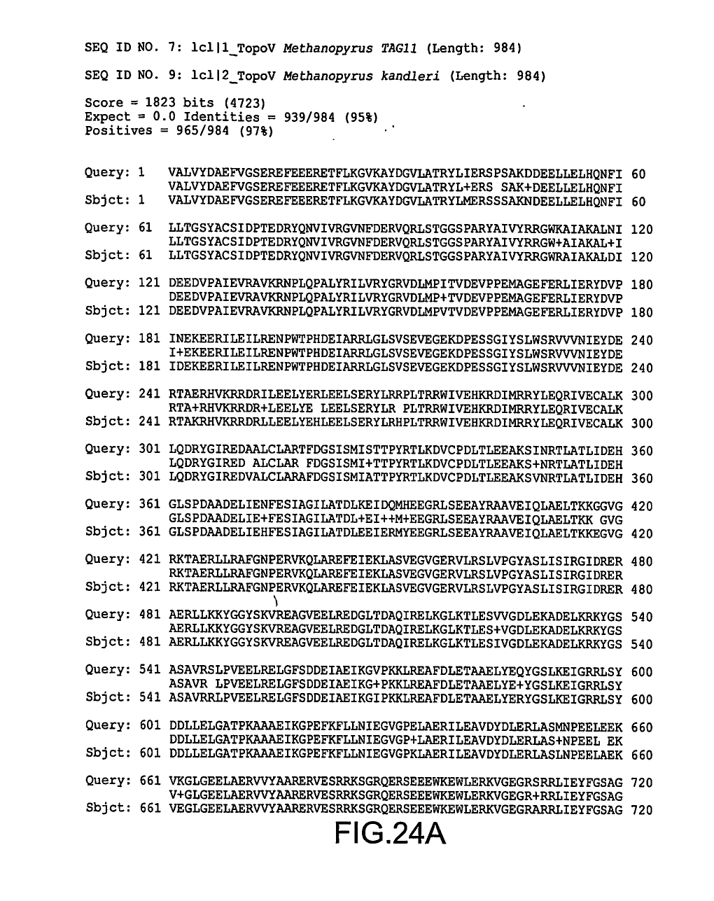

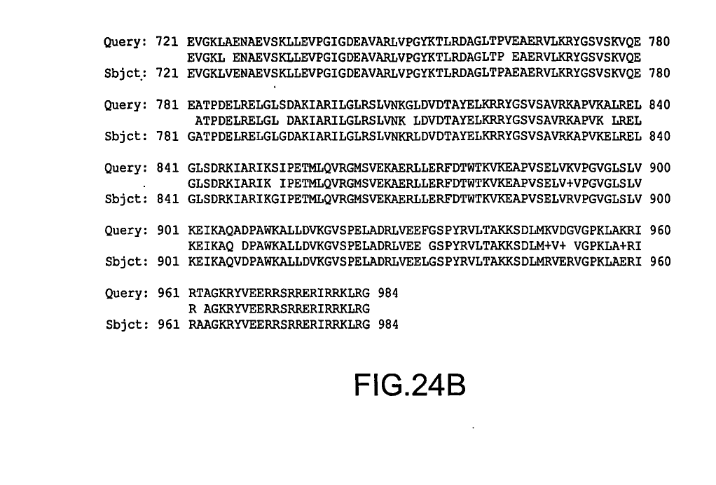

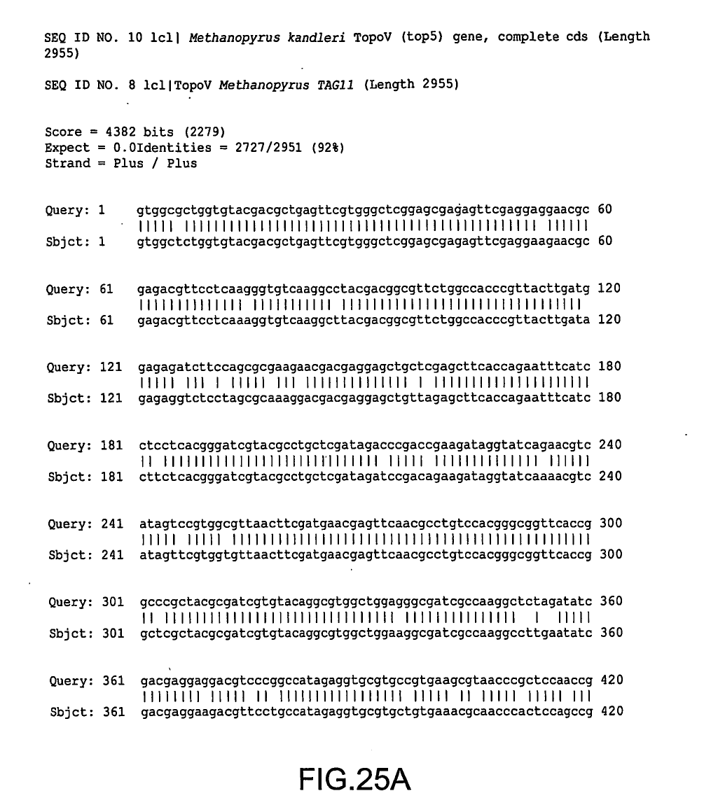

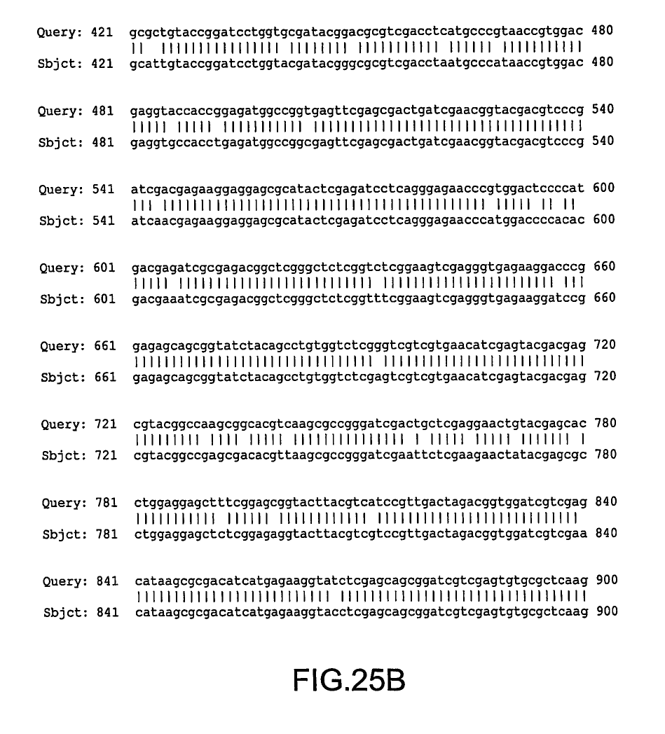

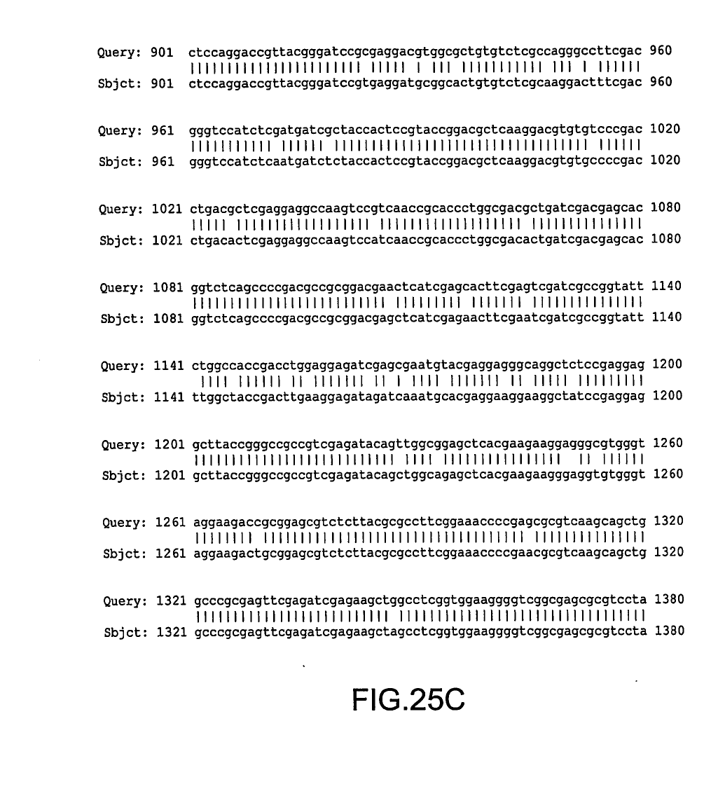

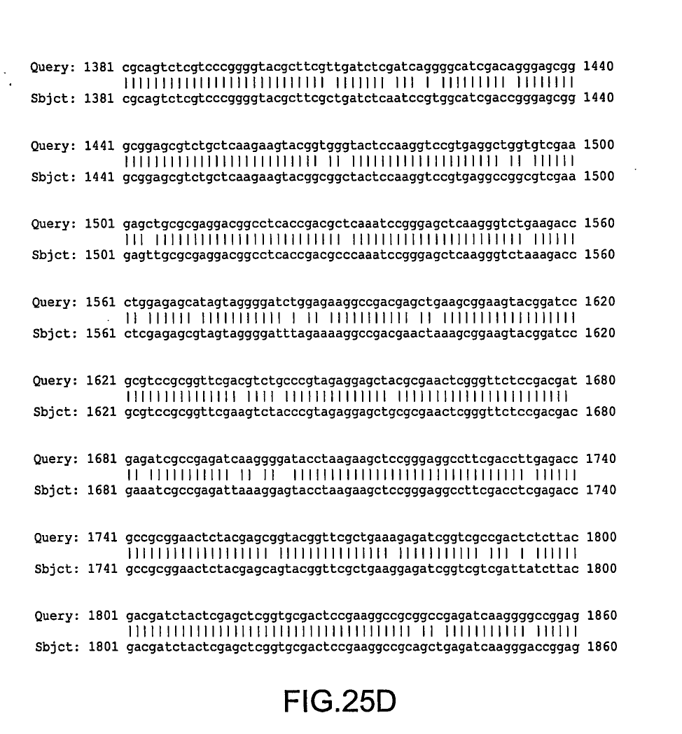

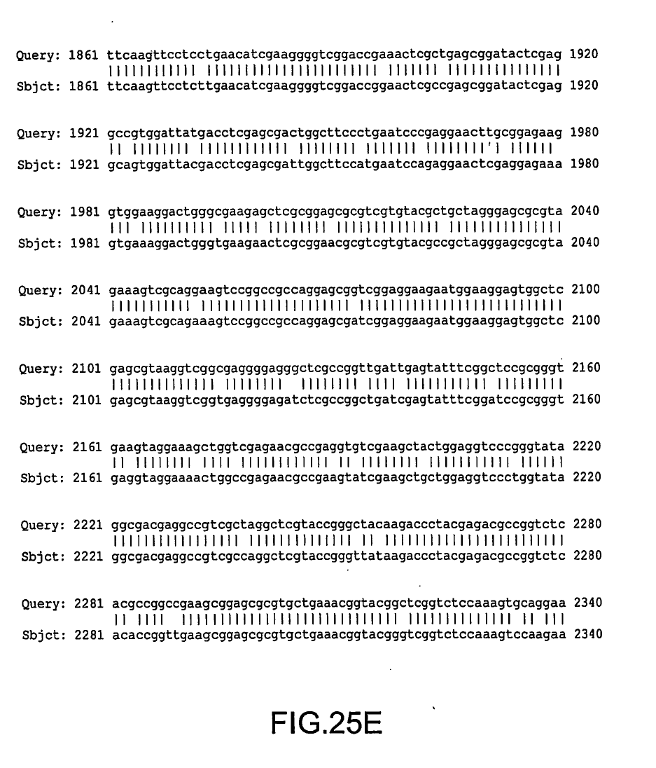

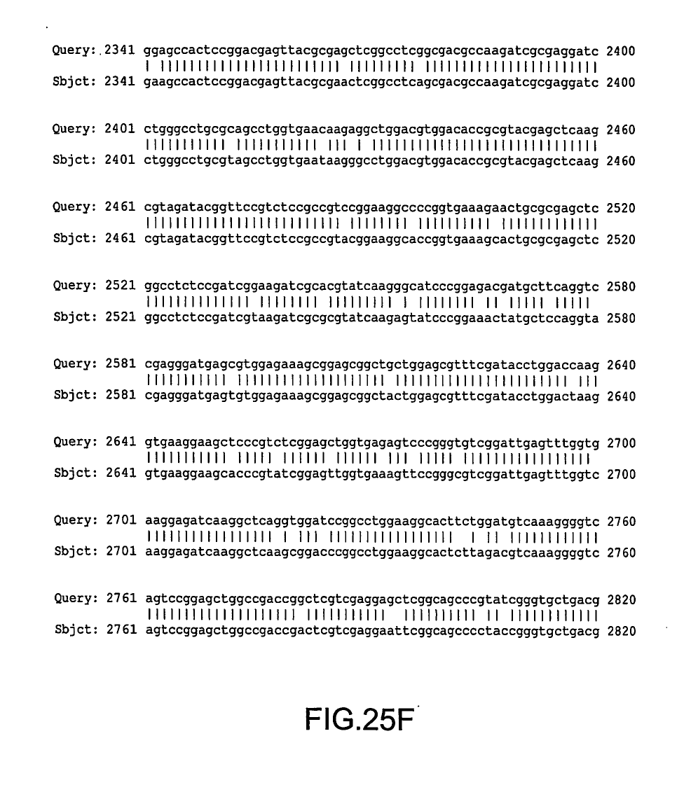

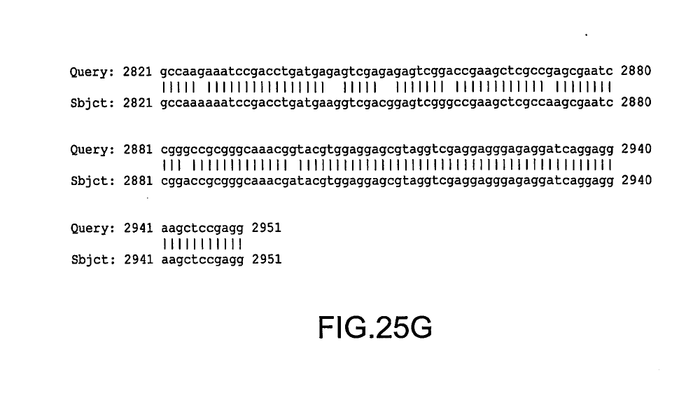

The amino acid sequence (SEQ ID NO. 7) and the nucleotide sequence (SEQ ID NO. 8) of the DNA Topoisomerase V from the second Methanopyrus species (Methanopyrus TAG11) were identified. The HhH-containing domain of Topoisomerase V from Methanopyrus KolB comprises amino acids 299-984. The amino acid sequence of the Methanopyrus KolB Topoisomerase V differs from its M. kandleri top5 counterpart by a few amino acid substitutions. FIGS. 24A-B show a comparison between the amino acid sequence (SEQ ID NO. 7) of the DNA topoisomerase V from Methanopyrus TAG11 and the amino acid sequence (SEQ ID NO. 9) of the DNA topoisomerase V from Methanopyrus kandleri top5. FIGS. 25A-G show a comparison between the nucleic acid sequence (SEQ ID NO. 8) of the DNA topoisomerase V from Methanopyrus TAG11 and nucleic acid sequence (SEQ ID NO. 10) of the DNA topoisomerase V from Methanopyrus kandleri top5.

SEQ ID NO.7:
VALVYDAEFVGSEREFEEERETFLKGVKAYDGVLATRYLIERSPSAKDDE
ELLELHQNFILLTGSYACSIDPTEDRYQNVIVRGVNFDERVQRLSTGGSP
ARYAIVYRRGWKAIAKALNIDEEDVPAIEVRAVKRNPLQPALYRILVRYG
RVDLMPITVDEVPPEMAGEFERLIERYDVPINEKEERILEILRENPWTPH
DEIARRLGLSVSEVEGEKDPESSGIYSLWSRVVVNIEYDERTAERHVKRR
DRILEELYERLEELSERYLRRPLTRRWIVEHKRDIMRRYLEQRIVECALK
LQDRYGIREDAALCLARTFDGSISMISTTPYRTLKDVCPDLTLEEAKSIN
RTLATLIDEHGLSPDAADELIENFESIAGILATDLKEIDQMHEEGRLSEE
AYRAAVEIQLAELTKKGGVGRKTAERLLRAFGNPERVKQLAREFEIEKLA
SVEGVGERVLRSLVPGYASLISIRGIDRERAERLLKKYGGYSKVREAGVE
ELREDGLTDAQIRELKGLKTLESVVGDLEKADELKRKYGSASAVRSLPVE
ELRELGFSDDEIAEIKGVPKKLREAFDLETAAELYEQYGSLKEIGRRLSY
DDLLELGATPKAAAEIKGPEFKFLLNIEGVGPELAERILEAVDYDLERLA
SMNPEELEEKVKGLGEELAERVVYAARERVESRRKSGRQERSEEEWKEWL
ERKVGEGRSRRLIEYFGSAGEVGKLAENAEVSKLLEVPGIGDEAVARLVP
GYKTLRDAGLTPVEAERVLKRYGSVSKVQEEATPDELRELGLSDAKIARI
LGLRSLVNKGLDVDTAYELKRRYGSVSAVRKAPVKALRELGLSDRKIARI
KSIPETMLQVRGMSVEKAERLLERFDTWTKVKEAPVSELVKVPGVGLSLV
KEIKAQADPAWKALLDVKGVSPELADRLVEEFGSPYRVLTAKKSDLMKVD
GVGPKLAKRIRTAGKRYVEERRSRRERIRRKLRG
SEQ ID NO.8:
GTGGCTCTGGTGTACGACGCTGAGTTCGTGGGCTCGGAGCGAGAGTTCGA
GGAAGAACGCGAGACGTTCCTCAAAGGTGTCAAGGCTTACGACGGCGTTC
TGGCCACCCGTTACTTGATAGAGAGGTCTCCTAGCGCAAAGGACGACGAG
GAGCTGTTAGAGCTTCACCAGAATTTCATCCTTCTCACGGGATCGTACGC
CTGCTCGATAGATCCGACAGAAGATAGGTATCAAAACGTCATAGTTCGTG
GTGTTAACTTCGATGAACGAGTTCAACGCCTGTCCACGGGCGGTTCACCG
GCTCGCTACGCGATCGTGTACAGGCGTGGCTGGAAGGCGATCGCCAAGGC
CTTGAATATCGACGAGGAAGACGTTCCTGCCATAGAGGTGCGTGCTGTGA
AACGCAACCCACTCCAGCCGGCATTGTACCGGATCCTGGTACGATACGGG
CGCGTCGACCTAATGCCCATAACCGTGGACGAGGTGCCACCTGAGATGGC
CGGCGAGTTCGAGCGACTGATCGAACGGTACGACGTCCCGATCAACGAGA
AGGAGGAGCGCATACTCGAGATCCTCAGGGAGAACCCATGGACCCCACAC
GACGAAATCGCGAGACGGCTCGGGCTCTCGGTTTCGGAAGTCGAGGGTGA
GAAGGATCCGGAGAGCAGCGGTATCTACAGCCTGTGGTCTCGAGTCGTCG
TGAACATCGAGTACGACGAGCGTACGGCCGAGCGACACGTTAAGCGCCGG
GATCGAATTCTCGAAGAACTATACGAGCGCCTGGAGGAGCTCTCGGAGAG
GTACTTACGTCGTCCGTTGACTAGACGGTGGATCGTCGAACATAAGCGCG
ACATCATGAGAAGGTACCTCGAGCAGCGGATCGTCGAGTGTGCGCTCAAG
CTCCAGGACCGTTACGGGATCCGTGAGGATGCGGCACTGTGTCTCGCAAG
GACTTTCGACGGGTCCATCTCAATGATCTCTACCACTCCGTACCGGACGC
TCAAGGACGTGTGCCCCGACCTGACACTCGAGGAGGCCAAGTCCATCAAC
CGCACCCTGGCGACACTGATCGACGAGCACGGTCTCAGCCCCGACGCCGC
GGACGAGCTCATCGAGAACTTCGAATCGATCGCCGGTATTTTGGCTACCG
ACTTGAAGGAGATAGATCAAATGCACGAGGAAGGAAGGCTATCCGAGGAG
GCTTACCGGGCCGCCGTCGAGATACAGCTGGCAGAGCTCACGAAGAAGGG
AGGTGTGGGTAGGAAGACTGCGGAGCGTCTCTTACGCGCCTUCGGAAACC
CCGAACGCGTCAAGCAGCTGGCCCGCGAGTTCGAGATCGAGAAGCTAGCC
TCGGTGGAAGGGGTCGGCGAGCGCGTCCTACGCAGTCTCGTCCCGGGGTA
CGCTTCGCTGATCTCAATCCGTGGCATCGACCGGGAGCGGGCGGAGCGTC
TGCTCAAGAAGTACGGCGGCTACTCCAAGGTCCGTGAGGCCGGCGTCGAA
GAGTTGCGCGAGGACGGCCTCACCGACGCCCAAATCCGGGAGCTCAAGGG
TCTAAAGACCCTCGAGAGCGTAGTAGGGGATTTAGAAAAGGCCGACGAAC
TAAAGCGGAAGTACGGATCCGCGTCCGCGGTTCGAAGTCTACCCGTAGAG
GAGCTGCGCGAACTCGGGTTCTCCGACGACGAAATCGCCGAGATTAAAGG
AGTACCTAAGAAGCTCCGGGAGGCCTTCGACCTCGAGACCGCCGCGGAAC
TCTACGAGCAGTACGGTTCGCTGAAGGAGATCGGTCGTCGATTATCTTAC
GACGATCTACTCGAGCTCGGTGCGACTCCGAAGGCCGCAGCTGAGATCAA
GGGACCGGAGTTCAAGTTCCTCTTGAACATCGAAGGGGTCGGACCGGAAC
TCGCCGAGCGGATACTCGAGGCAGTGGATTACGACCTCGAGCGATTGGCT
TCCATGAATCCAGAGGAACTCGAGGAGAAAGTGAAAGGACTGGGTGAAGA
ACTCGCGGAACGCGTCGTGTACGCCGCTAGGGAGCGCGTAGAAAGTCGCA
GAAAGTCCGGCCGCCAGGAGCGATCGGAGGAAGAATGGAAGGAGTGGCTC
GAGCGTAAGGTCGGTGAGGGGAGATCTCGCCGGCTGATCGAGTATTTCGG
ATCCGCGGGTGAGGTAGGAAAACTGGCCGAGAACGCCGAAGTATCGAAGC
TGCTGGAGGTCCCTGGTATAGGCGACGAGGCCGTCGCCAGGCTCGTACCG
GGTTATAAGACCCTACGAGACGCCGGTCTCACACCGGTTGAAGCGGAGCG
CGTGCTGAAACGGTACGGGTCGGTCTCCAAAGTCCAAGAAGAAGCCACTC
CGGACGAGTTACGCGAACTCGGCCTCAGCGACGCCAAGATCGCGAGGATC
CTGGGCCTGCGTAGCCTGGTGAATAAGGGCCTGGACGTGGACACCGCGTA
CGAGCTCAAGCGTAGATACGGTTCCGTCTCCGCCGTACGGAAGGCACCGG
TGAAAGCACTGCGCGAGCTCGGCCTCTCCGATCGTAAGATCGCGCGTATC
AAGAGTATCCCGGAAACTATGCTCCAGGTACGAGGGATGAGTGTGGAGAA
AGCGGAGCGGCTACTGGAGCGTTTCGATACCTGGACTAAGGTGAAGGAAG
CACCCGTATCGGAGTTGGTGAAAGTTCCGGGCGTCGGATTGAGTTTGGTC
AAGGAGATCAAGGCTCAAGCGGACCCGGCCTGGAAGGCACTCTTAGACGT
CAAAGGGGTCAGTCCGGAGCTGGCCGACCGACTCGTCGAGGAATTCGGCA
GCCCCTACCGGGTGCTGACGGCCAAAAAATCCGACCTGATGAAGGTCGAC
GGAGTCGGGCCGAAGCTCGCCAAGCGAATCCGGACCGCGGGCAAACGATA
CGTGGAGGAGCGTAGGTCGAGGAGGGAGAGGATCAGGAGGAAGCTCCGAG
GATGA

### EXAMPLE 11

### Dependence of Initial Rate of Primer Extension Reaction on Reaction Temperature

A primer extension assay for polymerization reaction at high temperatures was developed with a fluorescent duplex substrate containing a primer-template junction (PTJ). The duplex was prepared by annealing a 40-nt 5′-fluorescein end-labeled primer with a 50-nt long template:

```
  F1*
  |
gtgtggtggtggtggtggtggtggtggtggtggtggtggt
||||||||||||||||||||||||||||||||||||||||
cacaccaccaccaccaccaccaccaccaccaccaccaccaaaaaattttt
```

where SEQ ID NO. 9=gtgtggtggtggtggtggtggtggtggtggtggtggtggt and SEQ ID NO. 10=cacaccaccaccaccaccaccaccaccaccaccaccaccaaaaaattttt.

DNA polymerase reaction mixtures (5 μL) contained dATP, dTTP, dCTP, and dGTP (1 mM each), 4.5 mM MgCl₂, detergents Tween 20 and Nonidet P-40 (0.2% each), 0.32 μM PTJ, potassium glutamate, as indicated, and appropriate amounts of DNA polymerases in 30 mM Tris-HCl buffer pH 8.0 (25° C.). The background reaction mixtures contained all components except the polymerases. Primer extensions were carried out in PTC-150 Minicycler (MJ Research) for specified times in the range of temperatures 50-105° C. (with 5 degree intervals). For each reaction, 1.5 μL samples were removed after 3, 6, and 9 minutes of incubation and chilled to 4° C., followed by immediate addition of 20 μL of 20 mM EDTA. The samples were desalted by centrifugation through Sephadex G-50 spun columns, diluted, and analyzed on an ABI Prism 377 DNA sequencer (Applied BioSystems). For each sample, raw data were extracted from the sequencer trace files and the integral fluorescent intensities for each product peak were calculated. The total amount of fluorescent products for each time of incubation was determined, and the initial rates of extension were calculated from the progressive accumulation of the products as described earlier (Pavlov et al., 2002). For low temperatures of reaction (<70° C.) a regular 5′-fluorescein end-labeled PTJ substrate having the structure:

tttttgctgccggtcacttaacat

*F1-gtaatacgactcactataggg
|||||||||||||||||||||
tatgctgagtgatatcccaaaaaccccc

where SEQ ID NO. 11=gtaatacgactcactataggg and SEQ ID NO. 12=tttttgctgccggtcacttaacattatgctgagtgatatcccaaaaaccccc (Pavlov et al., 2002) was also used, and it gave virtually the same dependencies of changes in activity with temperature.

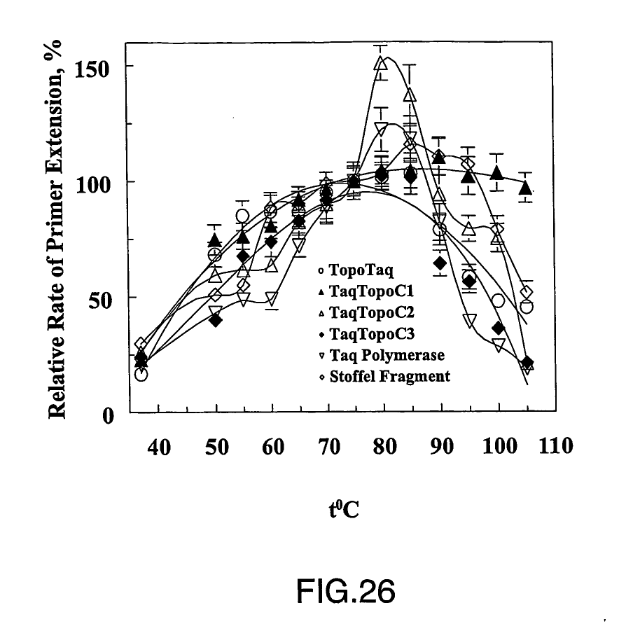

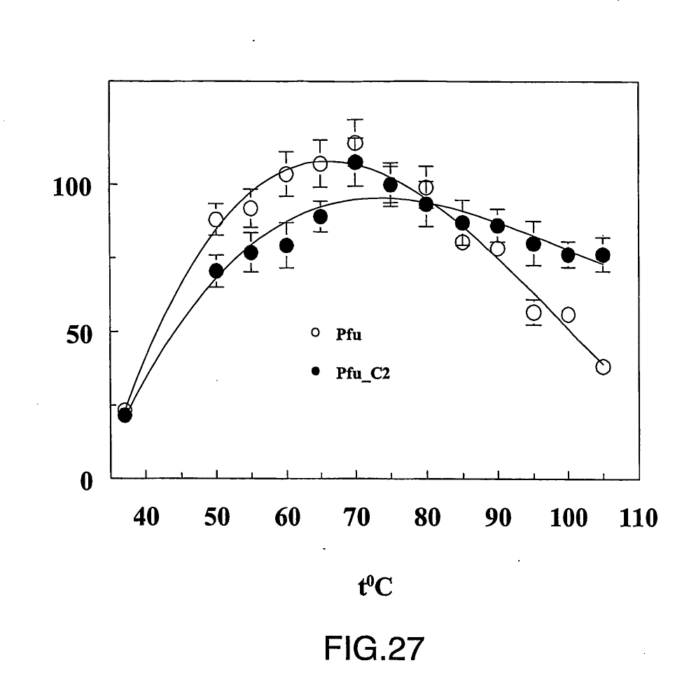

The results are summarized in FIGS. 26 and 27. FIGS. 26 and 27 are graphs illustrating the activity of Taq DNA polymerase, the Stoffel fragment, Pfu polB, and the hybrid polymerases at different temperatures. The dependencies of the initial rates of primer extension reactions on the reaction temperature were plotted for enzymes with Taq polymerase catalytic domain in FIG. 26, and for enzymes with Pfu polymerase catalytic domain in FIG. 27. The rates for TopoTaq, TaqTopoC1, TaqTopoC2, TaqTopoC3, and Pfu-C2 were measured in 0.25 M potassium glutamate; reactions with Pfu polymerase, Taq polymerase, and its Stoffel fragment were carried out without salts added. Replacement 0.25 M potassium glutamate by 0.25 M NaCl and 1M betaine resulted in the same activity for temperatures below 90° C. (not shown).

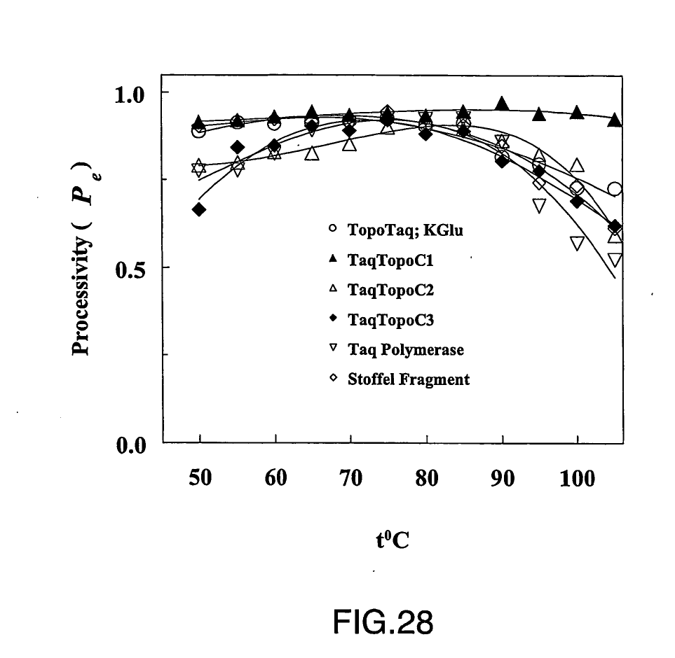

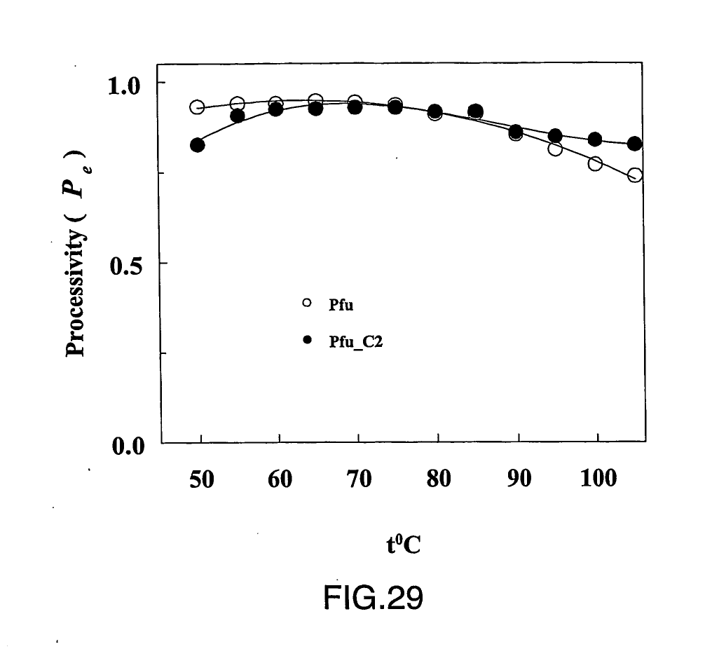

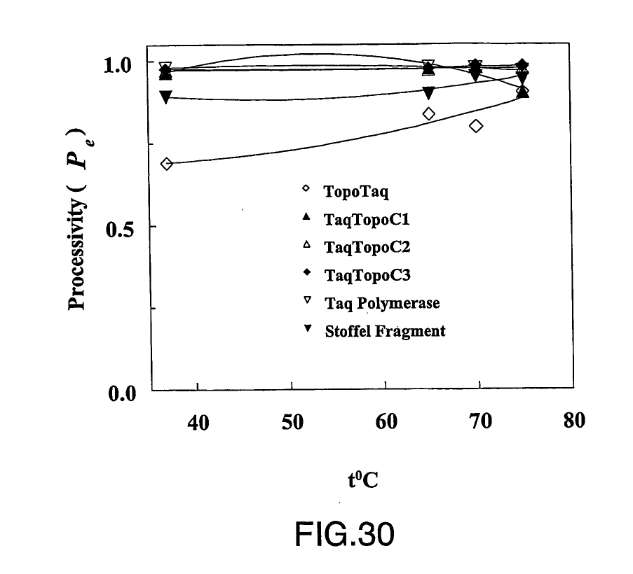

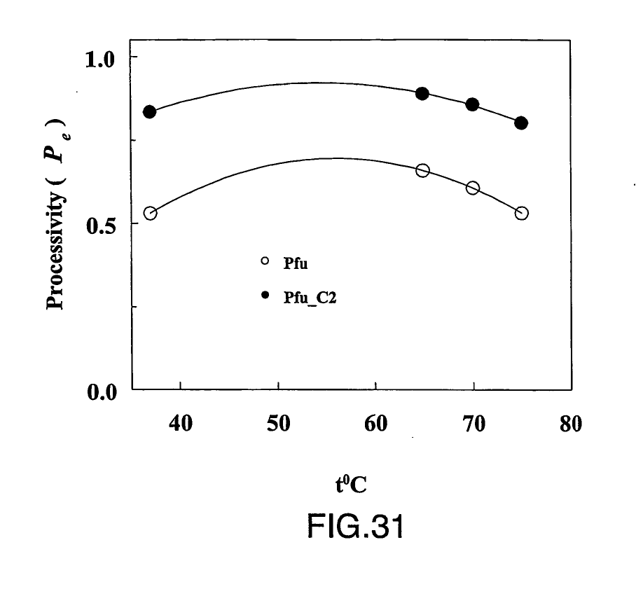

FIGS. 28-31 are graphs illustrating the processivity of Taq DNA polymerase, the Stoffel fragment, Pfu polB, and the hybrid polymerases at different temperatures. Processivities of enzymes in primer extension reactions were determined as described earlier (Pavlov et al., 2002). The dependencies were plotted for enzymes with Taq polymerase catalytic domain in FIGS. 28 and 30, and for enzymes with Pfu polymerase catalytic domain in FIGS. 29 and 31. Values for FIGS. 28 and 29 were obtained for the high-temperature substrate (see above); values for FIGS. 30 and 31 were measured for the regular substrate (Pavlov et al., 2002).

The results show that the catalytic domains of both Taq and Pfu DNA polymerases can carry out polymerization of DNA at temperatures as high as 105° C., with approximately the same efficiency as at lower temperatures. As shown in FIGS. 26 and 27, the activity at high temperatures is provided by DNA binding domains of Topo V properly positioned in the chimeras, rather than by addition of potassium glutamate. It is likely that the efficient binding of DNA at high temperatures occurs between double stranded part of PTJ and HhH motifs of superdomain C2 of Topo V. The results also imply that the rate of primer extension is much higher then the rate of melting the PTJ substrate even at the highest temperatures in the assay. It also suggests that DNA polymerases can stabilize PTJ duplexes during DNA synthesis.

The difference in processivity between Pfu DNA polymerase and PfuC2 chimera assumes that Pfu has lower efficiency in replication of G-C rich regions of DNA; attachment of TopoV DNA binding domains likely improves synthesis on these regions.

### EXAMPLE 12

### PCR Amplification of DNA by Chimeric DNA Polymerases

Due to attachment of extra DNA-binding domains, the chimeric DNA polymerases of this invention can perform DNA amplifications at high salt concentrations and in the presence of high levels of intercalating dyes and organic inhibitors. The chimeric DNA polymerases of this invention are ideally suited for demanding PCR applications that require robust amplification, such as DNA synthesis using templates from complicated media without additional purification or PCR from bacterial cultures. They also show excellent results when G-C rich DNA templates are used.

Amplification of Specific DNA Sequences

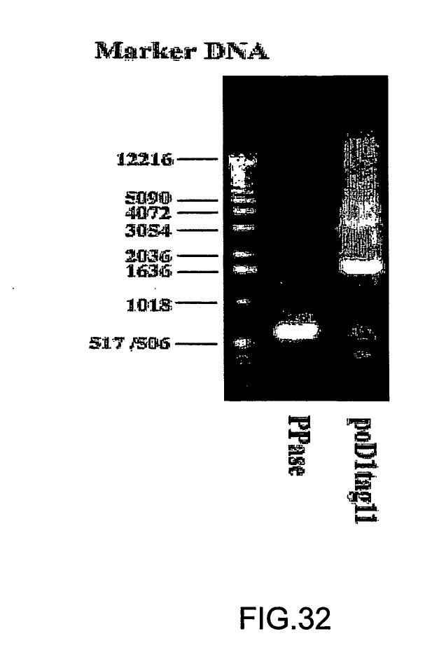

FIG. 32 is a gel image after amplification of specific DNA sequences cloned into plasmid pet21d by 0.07 units/μL TaqTopoC2 DNA polymerase. PCRs were carried out in 1× Amplification buffer (10 mM Tris-HCl, 3 mM MgCl₂, 50 mM potassium glutamate, 1M betaine, 0.06% Tween 20 and Nonidet NP40 each, and 12% trehalose) in the presence of T7 Promotor and T7 Terminator primers (0.3 μM each), dNTPs (0.3 mM each). Thirty (30) cycles were performed as follows: 100° C. for 40 seconds; 50° C. for 30 seconds; 72° C. for 2 minutes. The products were resolved on a 1% agarose gels and stained with ethidium bromide.

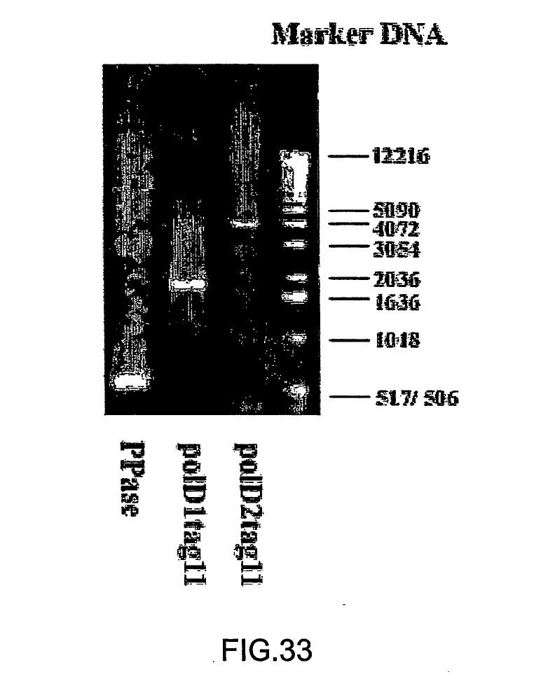

FIG. 33 is a gel image after amplification of specific DNA sequences cloned into plasmid pet21d by 0.07 units/μL Pfu-C2 DNA polymerase. PCRs were carried out in 1×Pfu PCR buffer (Stratagene) in the presence of T7 Promotor and T7 Terminator primers (0.3 μM each), dNTPs (0.3 mM each). Thirty (30) cycles were performed as follows: 100° C. for 40 seconds; 50° C. for 30 seconds; 72° C. for 2 minutes The products were resolved on a 1% agarose gels and stained with ethidium bromide.

### EXAMPLE 13

### Amplification at Very High Level of Salts

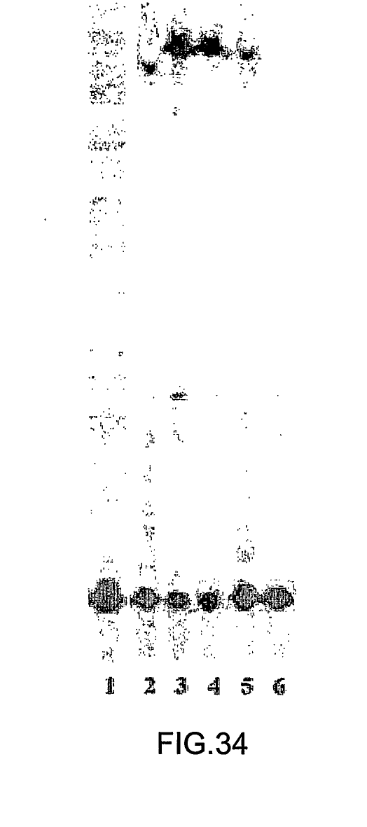

FIG. 34 is an image of a gel after amplification of 110 nt region of ssDNA M13mp18(+) with ALF M13 Universal fluorescent primer (Amersham Pharmacia Biotech) and primer caggaaacagctatgacc (SEQ ID NO. 13) (M13 reverse) in the presence of 0.25 M NaCl. The DNA polymerases used were: 1) AmpliTaq (Applied Biosystems); 2) TopoTaq; 3) TaqTopoC1; 4) TaqTopoC2; 5) Pfu-C2; and 6) Pfu cloned (Stratagene). Cycling (1 and 6): 94° C. for 40 seconds; 50° C. for 40 seconds; 72° C. for 2 minutes; 30 cycles. Cycling (2-5): 100° C. for 40 seconds; 50° C. for 30 seconds; 72° C. for 2 minutes; 30 cycles. PCR was conducted in 50 mM Tris-HCl buffer containing 50 mM potassium glutamate, 12% trehalose, and 1 M betaine (1-4) and in 1×PCR buffer for Pfu DNA polymerase (Stratagene). Reactions contained 500 μM dNTP (each) and 3.5 mM MgCl₂ (1-4) or 2 mM MgSO₄ and 1.5 mM MgCl₂ (5,6). The products were resolved on a 10% sequencing gel with ABI PRISM 377 DNA sequencer.

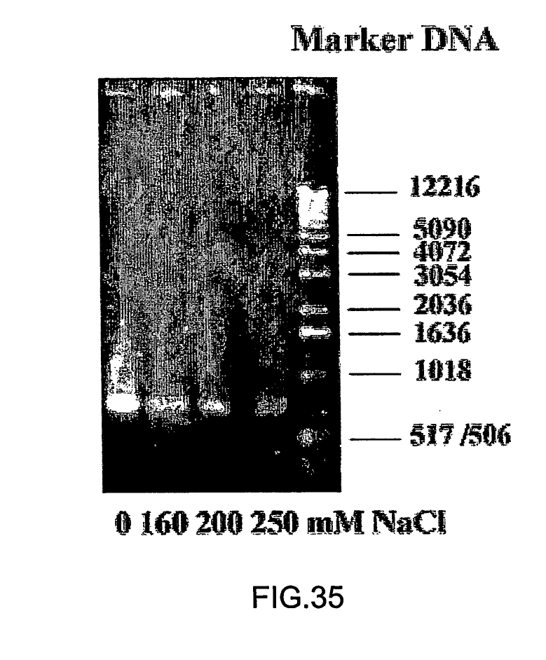

FIG. 35 is a gel image after amplification of 0.55 kb DNA cloned into plasmid pet21d (1 ng/μL reaction mixture) by TaqTopoC2 DNA polymerase in the presence of NaCl. PCRs were carried out in 1× Amplification buffer (10 mM Tris-HCl, 3 mM MgCl₂, 50 mM potassium glutamate, 1M betaine, 0.06% Tween 20 and Nonidet NP40 each, and 12% trehalose) in the presence of T7 Promotor and T7 Terminator primers (0.3 μM each), dNTPs (0.3 mM each), 0.05 units/μL TaqTopoC2 DNA polymerase and concentration NaCl indicated. Cycling: 100° C. for 40 seconds; 50° C. for 30 seconds; 72° C. for 2 minutes; 30 cycles. The products were resolved on a 1% agarose gels and stained with ethidium bromide.

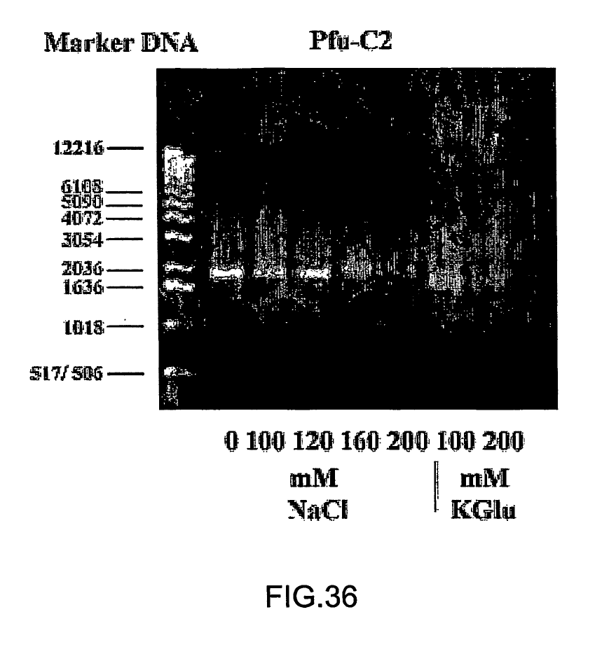

FIG. 36 is an image of a gel after amplification of 1.8 kb DNA cloned into plasmid pet21d (1 ng/μL reaction mixture) by Pfu-C2 DNA polymerase in salts. PCRs were carried out in 1×Pfu PCR buffer (Stratagene) in the presence of T7 Promotor and T7 Terminator primers (0.3 μM each), dNTPs (0.3 mM each), 0.07 units/μL Pfu-C2 DNA polymerase, and concentrations of salts indicated. Cycling: 100° C. for 40 seconds; 50° C. for 30 seconds; 72° C. for 2 minutes; 30 cycles. The products were resolved on a 1% agarose gels and stained with ethidium bromide.

### EXAMPLE 14

### Amplification in the Presence of Intercalating Dyes

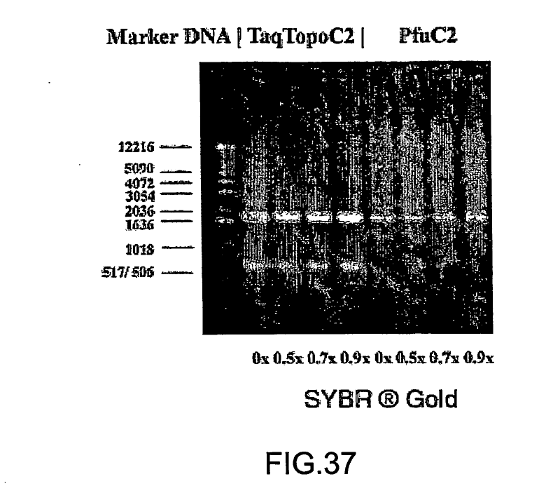

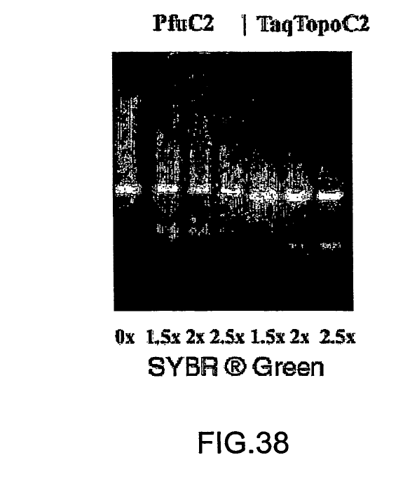

FIGS. 37 and 38 are gel images after amplification of 1.8 kb DNA cloned into plasmid pet21d (1 ng/μL reaction mixture) by TaqTopoC2 and PfuC2 DNA polymerases in the presence of SYBR® Gold Nucleic Acid Gel Stain (S-11494) (FIG. 37) and SYBR® Green I Nucleic Acid Gel Stain (Molecular Probes, Inc., Eugene, Oreg.) (FIG. 38). Cycling: 100° C. for 40 seconds; 50° C. for 30 seconds; 72° C. for 2 minutes; 30 cycles. The products were resolved on a 1% agarose gels and stained with ethidium bromide. Length of the in products is indicated on the traces.

### EXAMPLE 15

### Amplification of G-C-Rich Regions of Genomic DNA

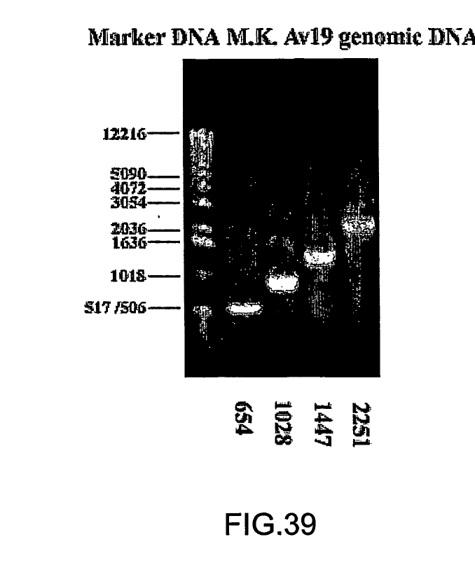

FIG. 39 is a gel image after amplification of regions of G-C-rich genomic DNA (10 ng/μL reaction mixture) by 0.05 units/μL TaqTopoC2 DNA polymerase (carried out in 1× Amplification buffer: 10 mM Tris-HCl, 3 mM MgCl₂, 50 mM potassium glutamate, 1M betaine, 0.06% Tween 20 and Nonidet NP40 each, and 12% trehalose) in the presence of T7 Promotor and T7 Terminator primers (0.3 μM each), dNTPs (0.3 mM each). Cycling: 100° C. for 40 seconds; 50° C. for 30 seconds; 72° C. for 2 minutes; 30 cycles. The products were resolved on a 1% agarose gels and stained with ethidium bromide. Length of the in products is indicated on the traces in FIG. 37.

### EXAMPLE 16

### Amplification of DNA from Bacterial Cultures

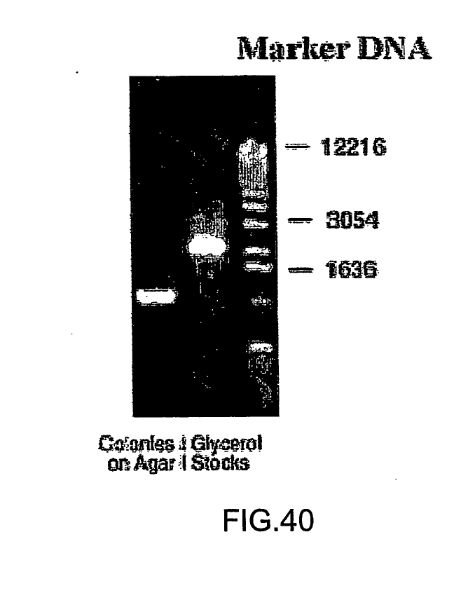

FIG. 40 is a gel image after amplification of plasmid DNA from E. coli bacterial culture by 0.05 units/μL TaqTopoC2 DNA polymerase (carried out in 1× Amplification buffer: 10 mM Tris-HCl, 3 mM MgCl₂, 50 mM potassium glutamate, 1M betaine, 0.06% Tween 20 and Nonidet NP40 each, and 12% trehalose) in the presence of T7 Promotor and T7 Terminator primers (0.3 μM each), dNTPs (0.3 mM each). Cycling: 100° C. for 40 seconds; 50° C. for 30 seconds; 72° C. for 2 minutes; 30 cycles. The products were resolved on a 1% agarose gels and stained with ethidium bromide.

### EXAMPLE 17

### Amplification of DNA in the Presence of High Concentration of Blood

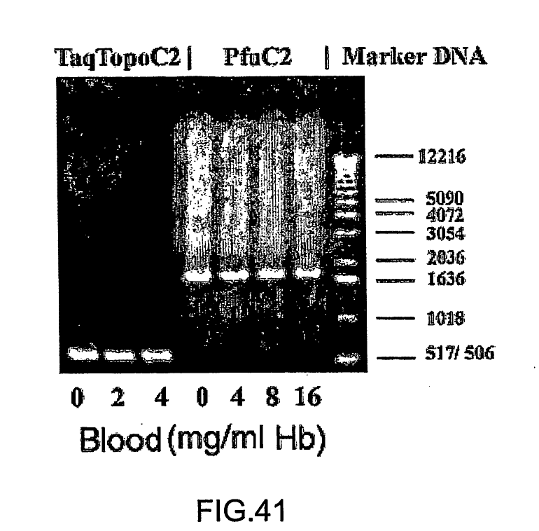

FIG. 41 is a gel image after amplification of 5.5 kb DNA cloned into plasmid pet21d (ng/μL reaction mixture) by 0.05 units/μL TaqTopoC2 DNA polymerase (carried out in 1× Amplification buffer: 10 mM Tris-HCl, 3 mM MgCl₂, 50 mM potassium glutamate, 1M betaine, 0.06% Tween 20 and Nonidet NP40 each, and 12% trehalose) and 1.8 kb DNA cloned into plasmid pet21d (1 ng/μL reaction mixture) by 0.07 units/μL Pfu-C2 DNA polymerase (carried out in 1×Pfu PCR buffer (Stratagene)). PCRs were performed in the presence of T7 Promotor and T7 Terminator primers (0.3 μM each), dNTPs (0.3 mM each), and blood (0 to 16 mg/mL Hb). Cycling: 100° C. for 40 seconds; 50° C. for 30 seconds; 72° C. for 2 minutes; 30 cycles. The products were resolved on a 1% agarose gels and stained with ethidium bromide.

### EXAMPLE 18

### Isothermic PCR Amplification of DNA by Chimeric DNA Polymerases

Due to DNA binding by TopoV domains and stabilization of the duplex substrate, the chimeric DNA polymerases are able to carry out DNA amplification at temperatures higher than suffice for melting double stranded DNA (FIGS. 26 and 27). This allows the amplification of DNA by PCR to be conducted at a constant temperature as described below.

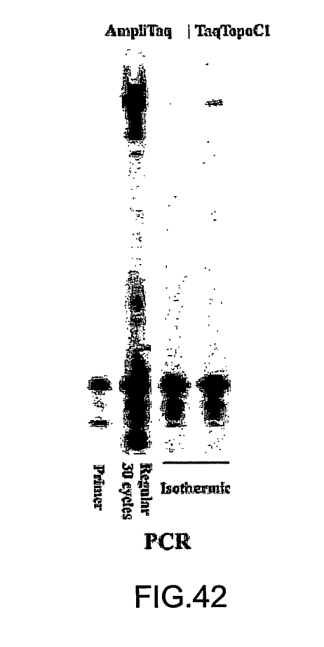

FIG. 42 is a gel image after amplification of a 110 nt region of ssDNA M13mp18(+) with ALF M13 Universal fluorescent primer (Amersham Pharmacia Biotech) and primer caggaaacagctatgacc (SEQ ID NO. 13) (M13 reverse). The polymerases used are shown in the figure. PCR was conducted in 50 mM Tris-HCl buffer containing 50 mM potassium glutamate, 12% trehalose, and 1 M betaine. Reactions contained 500 μM dNTP (each) and 3.5 mM MgCl₂. The products were resolved on a 10% sequencing gel with ABI PRISM 377 DNA sequencer. For regular PCR, the cycling was carried out as following: 94° C. for 40 seconds; 50° C. for 40 seconds; 72° C. for 2 minutes; 30 cycles (1, 6) or 100° C. for 40 seconds; 50° C. for 30 seconds; 72° C. for 2 minutes; 30 cycles. For isothermic PCR, after initial annealing of the primers to the template, the mixture was incubated at 75° C. for 2 h and then at 92° C. for 16 h.

### EXAMPLE 19

### Dye-Terminator Cycle Sequencing at High Salt Concentrations with the FS Mutant of TopoTaq

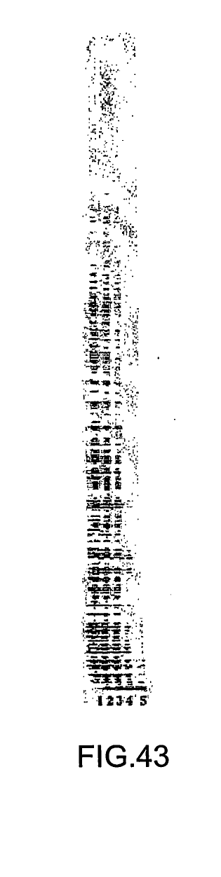

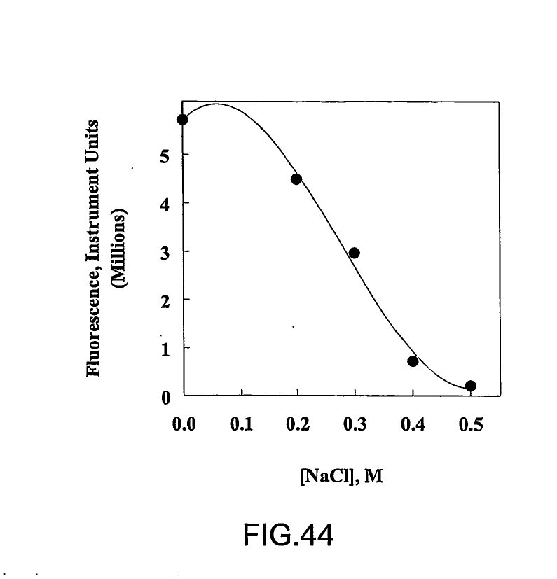

The FS mutant of TopoTaq had been prepared by substituting Phe683 in the TopoTaq sequence for Tyr using common PCR and cloning techniques and introduced into dye-terminator cycle sequencing reactions in the presence of increasing concentrations of NaCl. The incorporation of fluorescent nucleotides from the kit could be observed up to 0.4-0.5 M NaCl. No signal could be generated by the polymerase from the BigDye terminator kit even at the lowest salt concentration used. The results are shown in FIGS. 43 and 44. FIG. 43 is a urea gel analysis picture for M13mp18(+) sequencing reactions (50 ng ssDNA) with BDT v2 Kit, M13 Forward primer after 50 cycles. Tracks 1-5 show reactions carried out in the presence of 1 M betaine and 0, 0.2, 0.3, 0.4, and 0.5 M NaCl in the sequencing media, respectively. FIG. 44 is a graph of fluorescence versus NaCl concentration, illustrating how the integrated fluorescence intensity in the gel tracks decreases with increase of salt concentration.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims that follow.

### BIBLIOGRAPHY (ALL PUBLICATION LISTED ARE INCORPORATED HEREIN BY REFERENCE)

1. Harrison, S. C. (1991) Nature 353, 715-719.
2. Thayer, M. M., Ahern, H., Xing, D., Cunningham, R. P. and Tainer, I. A. (1995) Embo. J., 14, 4108-4120.
3. Pelletier, H., Sawaya, M. R., Woffle, W., Wilson, S. H. and Kraut, J. (1996) Biochemistry, 35, 12742-2761.
4. Doherty, A. J., Serpell, L. C. and Ponting, C. P. (1996) Nucleic Acids Res. 24, 2488-2497.
5. Shao, X. and Grishin, N. V. (2000) Nucleic Acids Res., 28, 2643-2650.
6. Slesarev, A. I., Stetter, K. O., Lake, J. A., Gellert, M., Krah, R. and Kozyavkin, S. A. (1993) Nature, 364, 735-737.
7. Slesarev, A. I., Lake, J. A., Stetter, K. O., Gellert, M. and Kozyavkin, S. A. (1994) J. Biol. Chem., 269, 3295-3303.
8. Slesarev, A. I., Belova, G. I., Lake, J. A. and Kozyavkin, S. A. (2001) Methods Enzymol., 334, 179-192.
9. Belova, G. I., Prasad, R., Kozyavkin, S. A., Lake, J. A., Wilson, S. H. and Slesarev, A. I. (2001) Proc Natl. Acad. Sci. USA, 98, 6015-6020.
10. Be1ova, G. I., Prasad, R., Nazimov, I. V., Wilson, S. H. and Slesarev, A. I. (2002) J. Biol. Chem., 277, 4959-4965.
11. Bedford, E., Tabor, S. and Richardson, C. C. (1997) Proc. Natl. Acad. Sci. USA, 94, 479-484.
12. Press, W. H., F1annery, B. P., Teukolsky, S. A., and Vetterling, W. T. (1986) Numeric Recipes: The Art of Scientific Computing (Cambridge University Press, Cambridge, United Kingdom).
13. Boeker, E. A. (1982) Biochem. J., 203, 117-123.
14. Washington, M. T., Johnson, R. E., Prakash, S. and Prakash, L. (1999) J. Biol. Chem., 274, 36835-36838.
15. Kim, Y., Eom, S. H., Wang, J., Lee, D. S., Suh, S. W. and Steitz, T. A. (1995) Nature, 376, 612-616.
16. Gelfand, D. H. and White, T. J. (1990) in PCR Protocols A Guide to Methods and Applications, ed. Innis, M. A., Gelfand, D. H., Sninsky, J. J., and White, T. J. (Academic Press, NY).
17. Barnes, W. M. (1992) Gene, 112, 29-35.
18. Kozyavkin, S. A., Pushkin, A. V., Eiserling, P. A., Stetter, K. O., Lake, J. A. and Slesarev, A. I. (1995) J. Biol. Chem., 270, 13593-13595.
19. Rees, W. A., Yager, T. D., Korte, J. and von Hippel, P. H. (1993) Biochemistry, 32, 137-144.
20. Melchior, W. B., Jr. and von Hippel, P. H. (1973) Proc. Natl. Acad. Sci. USA, 70, 298-302.
21. Fairfield, F. R., Newport, J. W., Dolejsi, M. K. and von Hippel, P. H. (1983) J. Biomol. Struct. Dyn., 1, 715-727.
22. von Hippel, P. H., Fairfield, F. R. and Dolejsi, M. K. (1994) Ann. N.Y. Acad. Sci., 726, 118-131.
23. Li, Y., Mitaxov, V. and Waksman, G. (1999) Proc. Natl. Acad. Sci. U.S.A., 96, 9491-9496.
24. Villbrandt, B., Sobek, H., Frey, B. and Schomburg, D. (2000) Protein Eng., 13, 645-454.
25. Innis, M. A., Myambo, K. B., Gelfand, D. H. and Brow, M. A. (1988) Proc. Natl. Acad. Sci. U.S.A., 85, 9436-9440.
26. Kaledin, A. S., Slyusarenko, A. G. and Gorodetskii, S. I. (1980) Biokhimiya, 45, 644-651.
27. Abu Al-Soud, W. and Radstrom, P. (1998) Appl. Environ. Microbiol., 64, 3748-3753.
28. Nixon, A. E., Ostermeier, M. and Benkovre, S. J. (1998) Trends Biotechnol, 16, 258-264
29. Peitsch, M. C. (1996) Biochem Soc Trans 24, 274-279.
30. Guex, N. & Peitsch, M. C. (1997) Electrophoresis 18, 2714-2723.
31. Guex, N., Diemand, A. & Peitsch, M. C. (1999) Trends Biochem. Sci. 24, 364-367.
32. Schwede, T., Diemand, A., Guex, N. and Peitsch, M. C. (2000) Res. Microbiol. 151, 107-112.
33. Combet, C., Jambon, M., Deleage, G. and Geourjon, C. (2002) Bioinformatics 18, 213-214.
34. Holm, L. & Sander, C. (1996) Nucleic Acids Res. 24, 206-9.
35. Brunne, R. M., van Gunsteren, W. F., R., B. and Ernst, R. R. (1993). J. Am. Chem. Soc. 115, 4764-4768.
36. Holm, L. and Sander, C. (1997) Nucleic Acids Res. 25, 231-4.
37. Gibrat, J. F., Madej, T. and Bryant, S. H. (1996) Curr. Opin. Struct. Biol. 6, 377-385.
38. Shindyalov, I. N. and Bourne, P. E. (1998) Protein Eng. 11, 739-747.

1. A chimeric DNA processing enzyme comprising a DNA processing enzyme or a fragment thereof linked to an amino acid sequence comprising one or more helix-hairpin-helix (HhH) motifs not naturally associated with said DNA processing enzyme or said fragment, wherein said amino acid sequence is derived from Topoisomerase V.

2. The chimeric DNA polymerase of claim 1, wherein said amino acid sequence comprises amino acid residues 685-984 of the M. kandleri top5 gene protein (SEQ ID NO:9).

3. The chimeric DNA polymerase of claim 1, wherein said amino acid sequence comprises amino acid residues 384-984 of the M. kandleri top5 gene protein (SEQ ID NO:9).

4. The chimeric DNA polymerase of claim 1, wherein said amino acid sequence comprises amino acid residues 518-984 of the M. kandleri top5 gene protein (SEQ ID NO:9).

5. The chimeric DNA polymerase of claim 1, wherein said amino acid sequence comprises amino acid residues 676-984 of the M. kandleri top5 gene protein (SEQ ID NO:9).

6. The chimeric DNA polymerase of claim 1, wherein said amino acid sequence is linked to the amino-terminus of said DNA polymerase.

7. The chimeric DNA polymerase of claim 1, wherein said amino acid sequence is linked to the COOH-terminus of said DNA polymerase.

8. The chimeric DNA polymerase of claim 1, wherein said DNA processing enzyme is Taq DNA polymerase.

9. The method of claim 1, wherein said fragment is a Stoffel fragment of Taq DNA polymerase.

10. The method of claim 1, wherein said DNA processing enzyme is Pfu polymerase.

11. A method of increasing the processivity of a DNA processing enzyme or a fragment thereof, comprising linking to said DNA processing enzyme or said fragment to an amino acid sequence comprising one or more helix-hairpin-helix (HhH) motifs not naturally associated with said DNA processing enzyme or said fragment, wherein said amino acid sequence is derived from Topoisomerase V.

12. The method of claim 11, wherein said DNA processing enzyme is Taq DNA polymerase.

13. The method of claim 11, wherein said DNA processing enzyme is Pfu polymerase.

14. A method of increasing the salt tolerance of a DNA processing enzyme or a fragment thereof in an amplification reaction, comprising linking to said DNA processing enzyme or said fragment to an amino acid sequence comprising one or more helix-hairpin-helix (HhH) motifs not naturally associated with said DNA processing enzyme or said fragment, wherein said amino acid sequence is derived from Topoisomerase V.

15. The method of claim 14, wherein said DNA processing enzyme is Taq DNA polymerase.

16. The method of claim 14, wherein said DNA processing enzyme is Pfu polymerase.

17. The method of claim 14, wherein said amplification reaction comprises a reaction mixture containing a salt in a concentration between about 0.5 and 2.0 M.

18. The method of claim 14, wherein said salt is selected from the group consisting of sodium chloride, potassium chloride, and potassium glutamate.

19. A method of increasing the thermal stability of a DNA processing enzyme or a fragment thereof, comprising linking to said DNA processing enzyme or said fragment to an amino acid sequence comprising one or more helix-hairpin-helix (HhH) motifs not naturally associated with said DNA processing enzyme or said fragment, wherein said amino acid sequence is derived from Topoisomerase V.

20. The method of claim 19, wherein said DNA processing enzyme is Taq DNA polymerase.

21. The method of claim 19, wherein said DNA processing enzyme is Pfu polymerase.

22. A method of amplifying a nucleic acid, comprising combining said nucleic acid with a chimeric DNA processing enzyme having a DNA processing enzyme or a fragment thereof linked to an amino acid sequence is derived from Topoisomerase V and contains one or more helix-hairpin-helix (HhH) motifs, wherein said nucleic acid and said chimeric DNA processing enzyme are combined in an amplification reaction mixture under conditions that allow amplification of said nucleic acid.

23. The method of claim 22, wherein said DNA processing enzyme is Taq DNA polymerase.

24. The method of claim 22, wherein said DNA processing enzyme is Pfu DNA polymerase.

25. The method of claim 22, wherein said reaction conditions comprise thermal cycling nucleic acid amplification conditions.

26. The method of claim 25, wherein said reaction conditions include a primer extension step that is carried out at a temperature between about 50 and 105° C.

27. The method of claim 22, wherein said reaction mixture comprises a high salt concentration.

28. The method of claim 22, wherein said reaction conditions include the addition of intercalating dyes to said reaction mixture.

29. The method of claim 22, wherein said reaction mixture includes a high concentration of blood.

30. The method of claim 22, wherein said nucleic acid includes G-C-rich regions.

31. The method of claim 22, wherein said nucleic acid is from bacteria.

32. The method of claim 31, wherein said bacteria is E. coli.

33. The method of claim 22, wherein said reaction conditions comprise isothermal amplification conditions.

34. The method of claim 32, wherein said reaction conditions include a primer extension step that is carried out at a constant temperature between about 50 and 105° C.

35. A chimeric DNA processing enzyme comprising a DNA processing enzyme or a fragment thereof linked to an amino acid sequence comprising one or more helix-hairpin-helix (HhH) motifs not naturally associated with said DNA processing enzyme or said fragment.
